(19) United States
(12) Patent Application Publication
(10) Pub. No.: US 2015/0353921 A9
(43) Pub. Date: Dec. 10, 2015

(54) METHOD OF ON-CHIP NUCLEIC ACID MOLECULE SYNTHESIS

(71) Applicant: Jingdong TIAN, Chapel Hill, NC (US)

(72) Inventor: Jingdong TIAN, Chapel Hill, NC (US)

(21) Appl. No.: 13/864,100

(22) Filed: Apr. 16, 2013

Related U.S. Application Data
(60) Provisional application No. 61/624,708, filed on Apr. 16, 2012.

(51) Int. Cl. C12N 15/10

(52) U.S. Cl. CPC C12N 15/1037

(57) ABSTRACT

A method of synthesizing a nucleic acid molecule, such as a gene, on a substrate or microchip is described. In particular, a method for synthesizing, amplifying, and assembling DNA oligonucleotides into a nucleic acid molecule or gene product, on a single substrate or microchip is described. Also described are a method of correcting a sequence error in a synthesized nucleic acid molecule, as well as a method for synthesizing and screening a library of codon variants to identify a nucleic acid molecule with an optimized level of protein expression.

### CROSS REFERENCE TO RELATED APPLICATIONS

This application is entitled to priority pursuant to 35 U.S.C. §119(e) to U.S. Provisional Application No. 61/624,708, filed on Apr. 16, 2012, the content of which is hereby incorporated by reference in its entirety.

### FIELD OF THE INVENTION

The present invention relates, in general, to nucleic acid molecule synthesis and, in particular, to a method comprising synthesizing and amplifying DNA oligonucleotides and assembling the oligonucleotides into a longer nucleic acid molecule, wherein the synthesis, amplification and assembly are effected on a solid substrate, such as a single microchip.

### BACKGROUND

High-throughput gene synthesis technology has been driven by recent advances in DNA microarrays that can produce pools of up to a million oligonucleotides for gene assembly (Tian et al, Mol. Biosyst. 5:714-722 (2009), Tian et al, Nature 432:1050-1054 (2004), Zhou et al, Nucleic Acids Res. 32:5409-5417 (2004), Richmond et al, Nucleic Acids Res. 32:5011-5018 (2004), Borovkov et al, Nucleic Acids Res. 38:e180 (2010)), albeit in minute quantities (105-106 molecules per sequence). The presence of too many oligonucleotide sequences in a pool makes it difficult to effectively use the entire oligonucleotide pool for gene assembly, as similar sequences can cross hybridize. Practical solutions include more efficient assembly strategies (Borovkov et al, Nucleic Acids Res. 38:e180 (201 0), Kosuri et al, Nat. Biotechnol. 28:1295-1299 (2010)), selective amplification of oligonucleotides (Kosuri et al, Nat. Biotechnol. 28:1295-1299 (2010)) or, as described herein, physical division of the oligonucleotide pool.

Furthermore, conventional strategies for high throughput gene synthesis that utilize DNA microarray technology allow for oligonucleotide synthesis on chip, however the oligonucleotides must be cleaved off of the chip for subsequent off-chip gene assembly, increasing the number of manipulations that must be performed on the oligonucleotide pool, which increases cost and decreases yield.

Removing errors that arise from oligonucleotide (oligo) synthesis and gene assembly also remains a significant challenge, especially for gene synthesis using microarray-produced oligonucleotides, where error rates tend to be higher (Tian et al, Nature 432:1050-1054 (2004), Borovkov et al, Nucleic Acids Res. 38:s180 Epub (2010)). A number of methods have been used to reduce synthesis errors. To improve the quality of gene-construction oligonucleotides, size exclusion purification using polyacrylamide gel electrophoresis (PAGE) (Ellington and Pollard, Jr., Curr. Protoc. Nucleic Acid Chern, Appendix 3, Appendix 3C), or high performance liquid chromatography (HPLC) (Andrus and Kuimelis, Curr. Protoc. Nucleic Acid Chern, Chapter 10, Unit 10 15) can be used to remove oligonucleotides that contain large insertions and deletions. An array hybridization method has also been developed to reduce errors in chip-generated oligo pools, which requires special microarrays of complementary oligonucleotides (Tian et al, Nature 432:1050-1054 (2004)). Methods of using mismatch-binding proteins (e.g. MutS) to remove error-containing DNA heteroduplexes have been developed (Can et al., Nucleic Acids Res. 2004; 32:e162; Smith et al., Proc. Natl. Acad. Sci. USA. 1997; 94:6847-6850; Binkowski et al., Nucleic Acids Res. 2005; 33:e55). However, MutS-based methods theoretically do not work well for error-rich sequences, because the correct sequences have to outnumber the erroneous sequences in order to avoid being depleted from the synthetic pool. A number of enzymes have been tested for enzymatic mismatch cleavage, including T7 endonuclease I, T4 endonuclease VII and Escherichia coli endonuclease V, which showed various effectiveness due to various specificities of the enzymes (Young et al., Nucleic Acids Res. 2004; 32:e59; Fuhrmann et al., Nucleic Acids Res. 2005; 33:e58; Bang et al., Nat. Methods. 2008; 5:37-39).

Thus, there exists a need for an improved method of high-throughput synthesis of nucleic acid molecules, and particularly for gene synthesis, wherein oligonucleotide synthesis, amplification and assembly into a single nucleic acid molecule, or gene, can be performed on a single chip. There also exists a need for a method of correcting sequence errors in nucleic acid molecules that may be introduced during high-throughput synthesis.

### BRIEF SUMMARY OF THE INVENTION

The present invention relates generally to synthesis of a nucleic acid molecule, such as a gene. More specifically, the invention relates to a method comprising synthesizing and amplifying DNA oligonucleotides and assembling the oligonucleotides into a longer nucleic acid molecule, such as a gene product, wherein the synthesis, amplification and assembly are effected in a single chamber on a single substrate, such as a microchip. The integration of oligonucleotide synthesis, amplification and assembly on the same substrate facilitates automation and miniaturization, which leads to cost reduction and increases the throughput of synthesis.

A method of gene synthesis according to an embodiment of the present invention is characterized in that isothermal nicking strand displacement amplification (nSDA) and polymerase cycling assembly reactions are performed on a single gene chip to achieve oligonucleotide amplification and gene assembly; the gene chip is formed by immobilizing or synthesizing oligonucleotides to the surface of a solid substrate.

Also disclosed is a method of effecting enzymatic error correction on synthetic genes. According to an embodiment of the present invention a mismatch-specific endonuclease is used in the error correction step, and the error correction step can be carried out on-chip or separately off-chip.

According to an embodiment of the present invention, a method of synthesizing a nucleic acid molecule having a target sequence comprises:
(1) obtaining a substrate having a chamber comprising a plurality of immobilized oligonucleotides for the synthesis of the target sequence,
(2) adding to the chamber a reaction mixture comprising dNTPs, a primer, a strand-displacing polymerase, a nicking endonuclease, a heat-stable DNA polymerase, and a buffer;
(3) amplifying the plurality of oligonucleotides to obtain free amplified oligonucleotides by a nicking strand displacement amplification reaction in the chamber containing the reaction mixture; and
(4) assembling the free amplified oligonucleotides to obtain the nucleic acid molecule by a polymerase cycling assembly reaction; wherein step (4) is conducted in the chamber without the need for a buffer change after step (3).

In a preferred embodiment, each of the plurality of oligonucleotides comprises a portion of the target sequence or a portion of the complementary sequence of the target sequence and a universal adaptor sequence at the 3′ end of the oligonucleotide for anchoring the oligonucleotide to the substrate surface.

In another preferred embodiment, the primer comprises a universal primer complementary to the universal adaptor sequence, the universal primer comprising a nucleotide sequence that is recognized and cut by the nicking endonuclease.

In yet another preferred embodiment, a method for synthesizing a nucleic acid molecule according to an embodiment of the present invention utilizes Bst DNA polymerase, large fragment, as the strand-displacing polymerase, and Nt.BstNBI as the nicking endonuclease, for the strand displacement amplification reaction, and Phusion polymerase as the heat-stable DNA polymerase for the polymerase cycling assembly reaction.

In another general aspect, the present invention provides a method for correcting a sequence error in a nucleic acid molecule synthesized according to a method of the present invention. According to embodiments of the present invention, the method comprises:
(1) heating and subsequently cooling a plurality of nucleic acid molecules synthesized according to a method of the present invention, thereby forming one or more heteroduplexes, wherein the heteroduplex comprises one or more mismatch sites resulting from the errors;
(2) contacting the one or more heteroduplexes with a mismatch-specific endonuclease under conditions for effective cleavage of the one or more heteroduplexes at the one or more mismatch sites, thereby obtaining cleaved fragments; and
(3) contacting the cleaved fragments with a DNA polymerase having 3′-5′ exonuclease activity under conditions for an overlap extension polymerase chain reaction amplification, thereby producing a plurality of nucleic acid molecules free of the one or more errors.

In a preferred embodiment, a CEL endonuclease, such as CEL II endonuclease from celery, and a proofreading DNA polymerase, such as Phusion polymerase, are used for the error correction.

In yet another general aspect, the present invention provides a method for screening a library of codon variants to obtain a nucleic acid sequence for optimized protein expression. According to embodiments of the present invention, the method comprises:
(1) synthesizing the library of codon variants using a method of synthesizing a nucleic acid molecule according to an embodiment of the present invention;
(2) amplifying the library by a polymerase chain reaction (PCR);
(3) operably linking the library of codon variants to a reporter gene sequence to obtain a library of reporter constructs;
(4) introducing the library of reporter constructs into a host cell; and
(5) measuring the expression of the reporter gene sequence from the host cell, thereby identifying the nucleic acid sequence for optimized protein expression.

In a particularly preferred embodiment, the present invention provides a method of on-chip synthesis for obtaining at least one synthesized gene. According to embodiments of the present invention, a method of on-chip synthesis of a gene comprises:
(1) obtaining a microchip comprising multiple chambers, each chamber comprising a plurality of immobilized oligonucleotides for the synthesis of a target sequence, wherein the target sequence comprises a fragment of the gene;
(2) adding to each of the chambers a reaction mixture comprising dNTPs, a primer, a strand-displacing polymerase, a nicking endonuclease, a heat-stable DNA polymerase, and a buffer;
(3) amplifying the plurality of oligonucleotides to obtain free amplified oligonucleotides by a nicking strand displacement amplification reaction in each of the chambers containing the reaction mixture;
(4) assembling the free amplified oligonucleotides to obtain the target sequence by a polymerase cycling assembly reaction, wherein step (4) is conducted in each of the chambers without the need for a buffer change after step (3).
(5) amplifying the target sequence from step (4) by a polymerase chain reaction (PCR) in each of the chambers;
(6) assembling the amplified target sequences from all chambers into a synthesized gene sequence; and
(7) correcting a sequence error in the synthesized gene sequence, comprising:
i. forming a heteroduplex comprising the synthesized gene sequence, the heteroduplex comprising one or more mismatch sites resulting from the sequence error;
ii. contacting the heteroduplex with a mismatch-specific endonuclease under conditions such that the heteroduplex is cleaved at the mismatch sites to obtain cleaved fragments of the gene; and
   iii. contacting the cleaved fragments with a DNA polymerase having 3′-5′ exonuclease activity under conditions for an overlap extension polymerase chain reaction amplification, thereby producing the gene sequence free of the sequence error.

The present invention also provides a kit for synthesizing a nucleic acid molecule, the kit comprising:
(1) a universal primer comprising a nucleotide sequence that is recognized and cut by a nicking endonuclease,
(2) the nicking endonuclease;
(3) a strand displacement DNA polymerase; and
(4) a DNA polymerase; and
(5) instructions on using the kit for synthesizing a nucleic acid molecule.

Preferably, the DNA polymerase has 3′-5′ exonuclease activity, and the kit further comprises a mismatch-specific endonuclease and additional instructions on enzymatic correction of sequence errors in the synthesized nucleic acid molecule.

In order for the aspects of the present invention to be more clearly understood, various embodiments will be further described in the following detailed description of the invention with reference to the accompanying drawings. The drawings and following detailed description are intended to provide examples of various embodiments of the present invention. It should be understood that the scope of the invention is not limited by the drawings and discussion of these specific embodiments.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

The patent or application file contains at least one drawing executed in color. Copies of this patent or patent application publication with color drawings will be provided by the Office upon request and payment of the necessary fee.

In the drawings:

FIG. 1 is a schematic representation of an integrated on-chip oligonucleotide array synthesis, amplification and nucleic acid assembly process according to an embodiment of the present invention: small pools of construction oligonucleotides are synthesized in separate chambers on a plastic DNA microchip using an inkjet DNA microarray synthesizer; the chambers are then filled with a combined amplification and assembly reaction mixture and sealed; in a nicking and strand displacement amplification reaction, a DNA polymerase (Bst large fragment shown in green) extends and displaces the preceding strand while a nicking endonuclease (Nt.BstNBI, shown in teal) separates the construction oligonucleotides from the universal primer (in red) and generates new 3′-ends for extension; and after amplification, the free oligonucleotides in each chamber are assembled into longer nucleic acid products by polymerase chain assembly;

FIGS. 2A and 2B show expression of synthetic lacZa codon variants in E. coli in a method of screening a library of codon variants according to an embodiment of the present invention: FIG. 2A shows a set of 1,296 E. coli colonies expressing distinct lacZa codon variants sorted by color intensity, raw images were acquired by scanning an agar plate on the scanning window of an HP Photosmart C7180 Flatbed Scanner; FIG. 2B are a bar graph and box plot showing distribution of color intensities of a different set of 1,468 random colonies expressing distinct lacZa codon variants on an agar plate; owing to the large size of the synthetic codon variant library, the chance of having identical clones on a plate was extremely low, as confirmed by sequencing several hundred blue colonies; in the box plot, the expression level of the wild-type (WT) lacZa is marked with a dash line;

FIG. 3A shows images of SDS-PAGE gels showing expression of 15 Drosophila transcription factor codon variants for identifying a codon variant with optimized protein expression according to an embodiment of the present invention: seventy-four Drosophila transcription factor gene fragments were optimized for protein production in E. coli by synthesizing 1,000-1,500 codon variants of each transcription factor, cloning each in frame with green fluorescent protein (GFP), and screening for the colonies with the highest fluorescence; expression data for 15 proteins is shown in FIG. 3A (see FIG. 7 for the remaining 59): each pair of lanes shows total cell protein extract of E. coli expressing the wild-type (left lane, WT) and optimized (right lane, Op) clones; equal amounts of the total cell protein extracts were separated on NuPage 4-12% gradient gels and stained with EZBlue Gel Staining Reagent; the broad bands marked by an arrow represent highly expressed transcription factor-GFP fusion proteins; there was no detectable expression of wild-type transcription factor-GFP fusion proteins as shown in the wild-type lanes; marker “M” lanes are Novex Prestained protein standards (Invitrogen);

FIGS. 4A and 4B are agarose gel images of the reaction product of an on chip nicked strand displacment amplification-polymerase cycling assembly (nSDA-PCA) reaction according to an embodiment of the invention: FIG. 4A is an agarose gel image of the nSDA-PCA reaction product showing as a typical smear (left lane) and the PCR amplified lacZa gene product (right lane), the middle lane is 100-bp DNA ladder; and FIG. 4B is an agarose gel image of the PCR amplification product of a gene encoding Red Fluorescent Protein (RFP) after an on-chip nSDA-PCA reaction followed by PCR amplification according to an embodiment of the invention;

FIGS. 5A and 5B are graphical representations of the statistical evaluation of errors in synthetic RFP genes with and without Surveyor nuclease treatment: FIG. 5A is a bar graph showing the percentage of fluorescent RFP colonies with and without error correction using Surveyor nuclease: on-chip RFP gene synthesis without error correction resulted in 50.2% fluorescent colonies while those treated with the Surveyor nuclease yielded an 84% fluorescent population, the total number of colonies in each population was approximately 3,000; and FIG. 5B is a graph showing the predicted correlation of the probability for an error-free clone as a function of product length (Fuhrmann et al, Nucl. Acids. Res. 33:e58 (2005)) before and after error correction with Surveyor nuclease: error correction using Surveyor nuclease (error frequency f_{c}=0.19 per kb, blue line) increases the probability of locating an error-free clone as compared to locating an error free clone among the uncorrected population (error frequency fᵤ=1.9 per kb, red line), thereby drastically reducing the number of colonies that need to be screened, the error frequencies were calculated from sequencing data in Table 1;

FIG. 6 shows compiled images of agarose gel electrophoresis of PCA assembled and PCR amplified codon libraries of 74 Drosophila transcription factor gene fragments, the lengths of the gene fragments fall in the range of 0.3-0.5 kb, and marker lane “M” is a 100-bp DNA ladder;

FIGS. 7A and 7B show images of SDS-PAGE gels showing expression of the remained 59 Drosophila transcription factor codon variants for identifying a codon variant with optimized protein expression according to an embodiment of the present invention, not shown in FIG. 3; FIG. 7A is an SDS-PAGE gel image showing the protein expression level of 59 transcription factor (TF) genes after codon optimization: highly expressed TF-GFP fusion protein bands were marked by arrows, total cell protein extracts were separated on NuPage 4-12% gradient gels (Invitrogen) and stained with EZBlue™ Gel Staining Reagent (Sigma), marker “M” lanes are Novex Prestained protein standards (Invitrogen); FIG. 7B is an SDS-PAGE gel image showing the intracellular processing of a TF-GFP fusion protein, and purification of a His-tagged TF antigen, the arrow marks a purified His6-tagged TF protein with the GFP fusion partner removed;

FIG. 8 is a schematic representation of the steps involved in a method of correcting a sequence error in a nucleic acid molecule according to an embodiment of the present invention: gene synthesis products are heat denatured and then slowly cooled down to form heteroduplexes containing mismatches at the error sites (left panel); heteroduplexes are cleaved by the Surveyor nuclease at the sites flanking the mismatch bulges; the resulting single-stranded overhangs, where mismatch bases are located, are removed by the proofreading exonuclease activity of Phusion polymerase used in the overlap-extension PCR (OE-PCR); the resulting fragments with mismatch bases removed are efficiently assembled back into full-length gene constructs during OE-PCR (right panel);

FIG. 9 is an image of an agarose gel showing the cleavage and reassembly of synthetic gene product during enzymatic error correction (ECR) according to a method of the present invention: synthetic rfp gene (lane 1) was incubated with Surveyor nuclease for 20 min (lane 2) and 60 min (lane 3) at 42° C., the cleavage reaction products (lane 2 and 3) were then re-assembled by OE-PCR into full-length gene products (lane 4 and 5, respectively, marked by arrow); the reaction products were analyzed by agarose gel electrophoresis (lane “M” is a DNA molecular weight marker);

FIGS. 10A and 10B are graphical representations of the results of an ECR performed according to an embodiment of the present invention, as measured by gene function or reporter assays: percentage of functional or fluorescent clones was measured before and after one or two iterations of ECR for 5 different gene constructs: FIG. 10A show the effects of Surveyor incubation time (20 and 60 min) and number of ECR iterations on the synthesis of an rfp gene with the correct sequence by counting fluorescent colonies; and FIG. 10B shows the percentage of blue (lacZa-v1&2) or fluorescent colonies (constructs-3&4) after 1 or 2 ECR iterations;

FIG. 11 is an agarose gel image showing RFP gene product that underwent ECR (incubated with Surveyor) followed by OE-PCR amplification according to an embodiment of the present invention, the amounts of nuclease and enhancer in Surveyor incubations were varied as follows: Lanes 1 and 4 (0.5 μl each), lanes 2 and 5 (1 μl each), and lanes 3 and 6 (2 μl each); the incubation temperature was also varied as follows: Lanes 1-3 (42° for 20 min) and lane 4-6 (25° for 60 min); results from all lanes appear similar, with no noticeable difference with increased enzyme amounts;

FIG. 12 show fluorescent images of agarose gel plates with colonies expressing synthesized RFP gene that underwent error correction according to an embodiment of the present invention: increased percentage of fluorescent RFP colonies was observed after employing error correction; images are examples of the increased fluorescent population derived by employing ECR with Surveyor nuclease; Panels A1 and A show colonies derived from the uncorrected RFP synthesis product that only yields 50.2% fluorescing colonies, iterations of correction, using 20 min incubations in this case, yield colonies with an increased fluorescent population as shown in Panels B1 and B (first iteration) and Panels C1 and C (second iteration); Panels A1, B1 and C1 are the same images as Panels A, B, and C with an added pseudo-colored red mask to highlight the brightly fluorescent colonies; and

FIGS. 13A and 13B are graphs showing the predicted effects of ECR as a function of sequence length: FIG. 13 A shows that the purity of gene synthesis products (as a percentage of error-free clones) decreases exponentially with the length of the product synthesized, employing ECR (1 error in 8,701 bp, blue line) dramatically increases the probability of locating an error-free clone as compared to locating an error-free clone in an uncorrected population (1 error in 526 bp, red line); FIG. 13B shows that employing ECR significantly reduces the number of colonies that need to be screened to have a high (95%) probability of obtaining at least one error-free clone, two iterations of 60 min cleavage incubations with Surveyor (blue line) could yield a correct 10 kb product by sequencing 8 random clones, plots are derived from the result of model calculations as described.

### DETAILED DESCRIPTION OF THE INVENTION

Unless defined otherwise, all technical and scientific terms used herein have the same meaning as is commonly understood by one of ordinary skill in the art to which this invention pertains. All publications and patents referred to herein are incorporated by reference. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

It must be noted that as used herein and in the appended claims, the singular forms “a,” “an,” and “the” include plural reference unless the context clearly dictates otherwise.

As used herein, the term “nucleic acid molecule” is intended to encompass any DNA molecule of interest, including but not limited to a naturally occurring gene, a synthetic gene, or a portion of a naturally occurring or synthetic gene. According to embodiments of the present invention, nucleic acid molecules can be obtained by any method known in the art in view of the present disclosure including, but not limited to, enzymatic methods, such as polymerase chain reaction (PCR) amplification, and chemical methods, such as de novo synthesis on-bead, on-chip gene synthesis, and DNA microarray synthesis.

As used herein, the term “gene” refers to a segment of DNA involved in producing a functional RNA. A gene can include the coding region, non-coding regions preceding (“5′UTR”) and following (“3′UTR”) the coding region, alone or in combination. The functional RNA can be an mRNA that is translated into a peptide, polypeptide, or protein. The functional RNA can also be a non-coding RNA that is not translated into a protein species, but has a physiological function otherwise. Examples of the non-coding RNA include, but are not limited to, a transfer RNA (tRNA), a ribosomal RNA (rRNA), a micro RNA, a ribozyme, etc. A “gene” can include intervening non-coding sequences (“introns”) between individual coding segments (“exons”). A “coding region” or “coding sequence” refers to the portion of a gene that is transcribed into an mRNA, which is translated into a polypeptide and the start and stop signals for the translation of the corresponding polypeptide via triplet-base codons. A “coding region” or “coding sequence” also refers to the portion of a gene that is transcribed into a non-coding but functional RNA.

As used herein, the terms “amplify,” “amplification,” and “amplifying” refer to the exponential or linear increase in the number of copies of a target nucleic acid sequence, such as an oligonucleotide, a double stranded or single-stranded nucleic acid molecule, a gene, a gene fragment, etc. Non-limiting examples of methods that can be used for amplifying nucleic acid sequences include polymerase chain reaction (PCR), strand-displacement amplification (SDA), polymerase cycling assembly (PCA), and overlap extension PCR (OE-PCR) amplification.)

As used herein, the term “primer” refers to a polynucleotide sequence that is complementary to a sequence on a target nucleic acid sequence and hybridizes to that sequence, serving as a point of initiation of nucleic acid synthesis, such as, for example, during an amplification reaction. The length and sequence of primers for use in amplification reactions can be designed based on principles known to those of ordinary skill in the art.

As used herein, the tem “oligonucleotide” or “oligo” refers to a single-stranded nucleic acid molecule that comprises the nucleotide sequence, or a portion of the sequence or complement thereof, of a target nucleic acid molecule to be synthesized.

As used herein, the term “microarray” or “microchip” refers to a substrate with a plurality of oligonucleotides immobilized to the surface of the substrate. A microarray can be physically divided into a plurality of “chambers” or “subarray.” According to an embodiment of the present invention, oligonucleotides within each chamber can be assembled together to form a longer nucleic acid molecule, such as a gene or a portion of a gene.

As used herein, the term “deoxyribonucleotides” or “dNTPs” refers to a mixture comprising adenine, guanine, thymine and cytosine nucleotide triphosphates used in an amplification reaction. Preferably all four nucleotide triphosphates are present in the mixture in equimolar amounts, however the molar amounts of each nucleotide triphosphate can be adjusted depending on the particular nucleotide sequence that is being amplified. For example, if the nucleotide sequence is GC rich, guanine triphosphate and cytosine triphosphate can comprise a larger molar fraction of the dNTP mixture than adenine and thymine triphosphate.

### Method of Synthesizing a Nucleic Acid Molecule

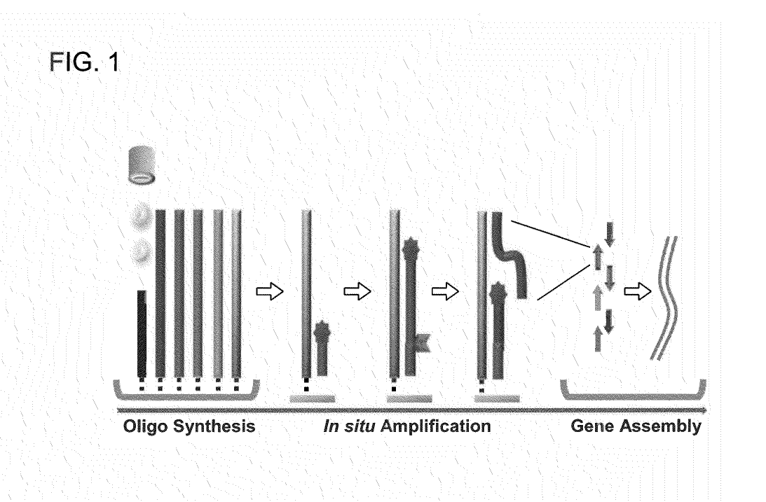

The present invention relates to a method of synthesizing a desired nucleic acid molecule. The synthesized nucleic acid molecule can be any desired DNA sequence, including but not limited to a naturally occurring gene, a synthetic gene, or portions of a naturally occurring or synthetic gene. Conventionally, chemical methods, such as NH₄OH treatment, have been used to cleave oligonucleotides from the substrate for subsequent gene assembly reactions, off-substrate. However, according to embodiments of the present invention, oligonucleotide synthesis, amplification and assembly into the nucleic acid molecule all occur on a single substrate, preferably within the same chamber of a substrate, without the need for changing buffers between the steps of oligonucleotide amplification and assembly (see FIG. 1).

Thus, in one general aspect, a method according to an embodiment of the present invention is characterized in that isothermal nicking strand displacement amplification (nSDA) reaction and polymerase cycling assembly (PCA) reactions are performed in a single chamber of a substrate to achieve oligonucleotide amplification and gene assembly without buffer change in between. According to embodiments of the present invention, each chamber contains a plurality of immobilized oligonucleotides that are used for the synthesis of a nucleic acid molecule (target sequence).

As used herein, the term “a plurality of immobilized oligonucleotides for the synthesis of a target sequence” refers to a collection of oligonucleotides that are immobilized to a substrate surface, that are subsequently amplified to obtain free amplified oligonucleotides that can be assembled into the target sequence using a method according to embodiments of the present invention. Each of the oligonucleotides comprises either a portion of the target sequence or a portion of the complementary sequence of the target sequence. The portion of the target sequence or its complementary sequence can be, for example, 40-300 bases in length, preferably, 48 to 200 bases in length, such as about 48, 54, 60, 72, 81, 90, 102, 111, 120, 132, 141, 150, 162, 171, 180, 192 or 198 bases in length. Each of the oligonucleotides comprises at least one region overlapping with a region on at least one other oligonucleotide. The overlapping region is about 15 to 35 bases in length, such as 15, 20, 30 or 35 bases in length. The oligonucleotides are able to tile the entire sequence of the target sequence, alternating between the sequence of the target sequence and its complementary sequence, via the complementary sequences in the overlapping regions of the oligonucleotides.

In addition to the portion of sequence designed to match the target sequence or complement thereof, an oligonucleotide according to an embodiment of the present invention can further comprise an universal adaptor at the 3′-end of the oligonucleotide.

As used herein, the term “universal adaptor” refers to a nucleotide sequence present at the 3′-end of each of a plurality of oligonucleotide. The universal adaptor comprises a nucleotide sequence for anchoring the oligonucleotide to a surface of substrate, and a nucleotide sequence that is recognized, but not cut, by a nicking endonuclease, such as, for example, Nt.BstNBI. The “universal adaptor” can be, for example, about 10 to about 100 bases in length, preferably, about 15 to 30 bases in length, such as about 15, 20, 25 or 30 bases in length.

Suitable substrates for use with the present invention include silicon, glass or plastic chips, slides or microscopic beads (see, for example, Ma et al, J. Mater. Chern. 19:7914-7920 (2009)). In a preferred embodiment of the present invention, cyclic olefin copolymer (COC) chips are used. In a most preferred embodiment, oligonucleotides are synthesized on the surface of a COC chip patterned with silicon spots using an inkjet microarray synthesizer.

Any method for synthesizing oligonucleotides on a substrate can be used in view of the present disclosure. Non-limiting examples for synthesizing oligonucleotides on a substrate include using an inkjet DNA microarray synthesizer (Saaem et al, ACS Applied Materials and Interface 2:491-497 (2010)). Microarray technologies that exist in the DNA synthesis market include, but are not limited to, ink-jet printing (Agilent, Protogene), photosensitive 5′ deprotection (Nimblegen, Affymetrix, Flexgen), photo-generated acid deprotection (Atactic/Xeotron/Invitrogen, LC Sciences), electrolytic acid/base arrays (Oxamer, Combimatrix/Customarray). (See also Tian et al, Mol Biosyst 5:714-722 (2009).) Other suitable methods include, e.g., printing with fine-pointed pins onto glass slides, photolithography using pre-made masks, photolithography using dynamic micromirror devices, or electrochemistry on microelectrode arrays.

Any method known in the art for immobilizing an oligonucleotide to the surface of a substrate can be used in view of the present disclosure. The oligonucleotides can be immobilized to the surface of the substrate in microscopic spots, with each spot containing oligonucleotides having the same sequence. The oligonucleotide can be anchored to the surface of the substrate using, for example, a standard chemical linker used in microarray synthesis. Non-limiting examples of standard chemical linkers include, but are not limited to, biotin, thiol group, alkynes, amino modifiers, and azide, etc. Typically, oligonucleotides are immobilized to the surface of a substrate via the 3′ end of the oligonucleotide. If the oligonucleotide is synthesized with a universal adaptor sequence, the oligonucleotide can be anchored to the surface of the substrate via the adaptor sequence using a standard chemical linker used in microarray synthesis.

According to embodiments of the present invention, the surface of a substrate can be partitioned into chambers, such that the resulting substrate comprises a plurality of chambers that are physically isolated. For example, the substrate can be partitioned using physical barriers, or by using differences in hydrophobic, where inside of the chamber is hydrophilic and the outside areas are hydrophobic. Each chamber can contain a plurality of spots, wherein each spot comprises a single set of oligonucleotide sequence. In this case, each chamber of a substrate can comprise only the oligonucleotides necessary for the assembly of a single nucleic acid molecule, with such oligonucleotides being physically separated from oligonucleotides in the other chambers of the microchip. An advantage of using a substrate with chambers is that each individual chamber only contains those oligonucleotides necessary for assembly of a single nucleic acid molecule, which can be about 0.5 to about 1 kb in length, allowing the oligonucleotides to be used more effectively. Because each chamber is physically isolated from all of the other chambers of the same substrate, the need for post-synthesis partitioning of the oligonucleotide pool by complex methods, such as microfluidic manipulation, is eliminated.

According to embodiments of the present invention, a standard 1″×3″ chip can be used for synthesizing a nucleic acid molecule according to an embodiment of the present invention. The surface of a 1″×3″ chip can be divided into as many as 30 chambers, or subarrays, each containing 361 spots for synthesizing a unique oligonucleotide sequence. Thus, using a method of the present invention for synthesizing a nucleic acid molecule, about 10,830 different oligonucleotide sequences having a length of 85 bases can be synthesized on a single substrate, providing a capacity to produce up to 30 kb of assembled nucleic acid molecules, or DNA.

According to a particular embodiment of the present invention, a substrate, such as a microchip, that is partitioned into a plurality of chambers can be used to synthesize a plurality of nucleic acid molecules simultaneously, wherein each of the plurality of nucleic acid molecules is synthesized in a separate chamber. In a particularly preferred embodiment, each chamber comprises the oligonucleotides necessary to assemble a longer nucleic acid molecule of, for example, about 0.5 to about 1.0 kb in length. However, longer sequences can be hierarchically assembled from 0.5 to 1.0 kb nucleic acid molecules obtained according to a method of the present invention.

According to embodiments of the present invention, the immobilized oligonucleotides are amplified on the substrate by a strand displacement amplification (SDA) reaction, and particularly by a nicking strand displacement amplification (nSDA) reaction, to yield amplified free oligonucleotides. As used herein, the terms “strand displacement amplification” and “nicking strand displacement amplification” all refer to an in vitro nucleic acid amplification reaction performed in the presence of a strand-displacing polymerase and a nicking endonuclease, wherein the nicking endonuclease creates a nick in a double stranded or partially double stranded nucleic acid, creating a free 3′ end from which a strand displacing polymerase can initiate synthesis of a new strand while simultaneously displacing the previously synthesized strand. SDA is an isothermal amplification reaction and thus does not require temperature changing.

As used herein a “nicking endonuclease” refers to an endonuclease that recognizes a nucleotide sequence of a completely or partially double-stranded nucleic acid molecule and cleaves only one strand of the nucleic acid molecule at a specific location relative to the recognition sequence. As used herein, the term “nick” or “nicking” refers to the cleavage of only one strand of a completely or partially double-stranded nucleic acid molecule at a specific position relative to a nucleotide sequence that is recognized by the nicking endonuclease performing the nicking, resulting in a 3′-hydroxyl and 5′-phosphate, which can serve as initiation points for a variety of further enzymatic reactions including strand-displacement amplification.

Any suitable nicking endonuclease can be used in the SDA according to embodiments of the present invention. Such nicking endonucleases include, but are not limited to, those available from New England BioLabs (Ipswich, Mass.), e.g., Nt.BspQI (NEB #R0644), a derivative of the restriction enzyme BSpQI (NEB #R0712) that cleaves one strand of DNA on a double-stranded DNA substrate; Nt.CviPII (NEB #R0626), a naturally occurring nicking endonuclease cloned from cholorella virus NYs-1; Nt.BstNBI (NEB #R0607), a naturally occurring thermostable nicking endonuclease cloned from Bacillus Stereothermophilus; Nb.BsrDI (NEB #R0648) and Nb.BtsI (NEB #R0707), naturally occurring large subunits of thermostable heterodimeric enzymes (5); Nt.AlwI (NEB #R0627), a derivative of the restriction enzyme AlwI (NEB #R0513), that has been engineered to behave in the same way, i.e., both nick just outside their recognition sequences; Nb.BbvCI (NEB #R0631) and Nt.BbvCI (NEB #R0632), alternative derivatives of the heterodimeric restriction enzyme BbvCI, each engineered to possess only one functioning catalytic site, and the two enzymes nick within the recognition sequence but on opposite strands; Nb.BsmI (NEB #R0706), a bottom-strand specific variant of BsmI (NEB #R0134) discovered from a library of random mutants.

In a preferred embodiment, the nicking endonuclease is Nt.BstNBI, which cleaves only one strand of DNA on a double-stranded DNA substrate. Nt.BstNBI catalyzes a single strand break 4 bases beyond the 3′ side of the recognition sequence.

As used herein, a “strand-displacing polymerase” refers to a DNA polymerase that is capable of initiating synthesis from the 3′ end of a nucleic acid at the site of a nick, and displacing the previously synthesized nucleic acid strand while synthesizing the new strand. Non-limiting examples of strand-displacing polymerases that can be used in a method of the present invention include Bst DNA polymerase (large fragment) (New England Biolabs), Sequenase™ (Affymetrix), and phi29 polymerase (New England Biolabs).

In a preferred embodiment, the strand-displacing polymerase is Bst large fragment, which is portion of the Bacillus stearothermophilus DNA Polymerase protein that contains the 5′→3′ polymerase activity, but lacks 5′→3′ exonuclease activity.

According to embodiments of the present invention, a reaction mixture for strand displacement amplification of the immobilized oligonucleotides comprises deoxynucleotide triphosphates (dNTPs), a primer, a strand-displacing polymerase, and a nicking endonuclease. In a particular embodiment, a substrate having a plurality of chambers is used and the strand displacement amplification can be initiated, for example, by filling each chamber with the strand-displacement amplification reaction mixture.

According to embodiments of the present invention, a primer comprises less than about 50 bases in length, and is preferably about 10-40 bases in length, and more preferably about 15-30 bases in length, and most preferably 20 bases in length.

According to a preferred embodiment, a primer used for strand-displacement amplification comprises a universal primer having a sequence that is complementary to the universal adaptor sequence. In another preferred embodiment, the universal primer comprises a nucleotide sequence that is recognized and cut by the nicking endonuclease used in the SDA, such as, for example, the recognition site of Nt.BstNBI.

In a particularly preferred embodiment, a primer comprises a universal primer complementary to the universal adaptor sequence, the universal primer comprising the recognition site of Nt.BstNBI.

The amplified oligonucleotides obtained from strand-displacement amplification are assembled together to obtain at least one of the nucleic acid molecule by a polymerase cycling assembly (PCA) reaction. According to embodiments of the present invention, the strand-displacement amplification reaction and polymerase cycling assembly reaction occur on a single substrate in the same buffer conditions. As used herein, the term “polymerase cycling assembly reaction” refers to a method of assembling a nucleic acid molecule from a plurality of oligonucleotide fragments of the nucleic acid molecule and the complementary sequence of the nucleic acid molecule, in the presence of a DNA polymerase enzyme, wherein each of the oligonucleotide fragments comprises at least one overlapping portion with at least one other oligonucleotide fragment. During the PCA, an overlapping region on one oligonucleotide fragment anneals to a complementary overlapping region on another oligonucleotide fragment, and the gaps between the annealed fragments are filled in by a DNA polymerase enzyme using the oligonucleotide fragments as the templates. Each cycle in the PCA increases the length of various fragments randomly depending on which oligonucleotides find each other. Preferably, the oligonucleotides, like the pair of primers used in regular PCR, have similar melting temperatures, are hairpin free and not too GC rich to avoid complications for the PCA.

Any DNA polymerase enzyme can be used for the polymerase cycling assembly reaction in a method of the present invention. Preferably, the DNA polymerase is a high-fidelity DNA polymerase, meaning that the DNA polymerase has a proof-reading function such that the probability of introducing a sequence error into the resulting, intact nucleic acid molecule is low. Examples of DNA polymerases suitable for the polymerase cycling assembly reaction include, but are not limited to Phusion polymerase, platinum Taq DNA polymerase High Fidelity (Invitrogen), Pfu DNA polymerase, etc.

As used herein, the term “Phusion polymerase” refers to thermal stable DNA polymerase that contains a Pyrococcus-like enzyme fused with a processivity-enhancing domain, resulting in increased fidelity and speed, e.g., with an error rate >50-fold lower than that of Tag DNA Polymerase and 6-fold lower than that of Pyrococcus furiosus DNA Polymerase. It possesses 5′→3′ polymerase activity, 3′→5′ exonuclease activity and will generate blunt-ended products. An example of Phusion polymerase is Phusion® High-Fidelity DNA Polymerase (New England Biolabs).

According to embodiments of the present invention, oligonucleotide amplification and assembly occur in a single chamber on a substrate without the need for buffer exchange. Thus in one embodiment, the nicking endonuclease, strand displacement polymerase, and DNA polymerase for the PCA reaction are added to a chamber of a substrate in a single reaction mixture, such that the PCA reaction can take place immediately after strand-displacement amplification. Because strand-displacement amplification is an isothermal amplification reaction and polymerase cycling assembly requires thermal cycling, after addition of a reaction mixture containing all the components necessary for both reactions, the temperature is held constant to allow for strand-displacement amplification, followed by switching the reaction mode to isothermal cycling to allow the polymerase cycling assembly reaction to take place.

As an illustrative and non-limiting example, a combined strand-displacement amplification and polymerase cycling assembly reaction can be carried out by incubating at 50° C. for 2 hours followed by 80° C. for 20 min (strand-displacement amplification), and then increasing the temperature to 98° C. for 30 sec, performing 40 cycles of denaturation at 98° C. for 7 sec, annealing at 60° C. for 60 sec, and elongation at 72° C. for 15 sec/kb, finishing with an extended elongation step at 72° C. for 5 min (polymerase cycling assembly).

One of ordinary skill in the art will recognize that the temperatures used for strand-displacement amplification, and denaturation, annealing, and elongation in the polymerase cycling assembly reaction, as well as the length of time for each step, will depend upon a variety of factors, including but not limited to, specific enzymes used, length of oligonucleotides to be amplified, length of nucleic acid molecule to be synthesized, and oligonucleotide sequence, and will be able to readily adjust such parameters in order to achieve the optimal results.

The efficiency and the obtained amount of amplified oligonucleotides from the strand displacement amplification and polymerase cycling assembly reactions can be affected by various reaction parameters, such as, for example, time, concentration of enzymes (nicking endonuclease, strand displacement polymerase, DNA polymerase etc.), concentration of dNTPs, and concentration of other buffer components including salts etc. In view of the present disclosure, one of ordinary skill in the art will be able to readily determine the optimal values for each of the various reaction parameters in order to optimize amplification and assembly of the oligonucleotides into the desired nucleic acid molecule. For example, it is estimated that a 2 hour reaction time results in an approximately 4-fold amplification. Thus, the extent of the amplification can be adjusted by controlling the reaction time, and is preferably adjusted such that the amplification is linear so as to keep the ratios constant among amplified oligonucleotides.

In a particularly preferred embodiment, a reaction mixture for the combined SDA and PCA reactions comprises a universal primer comprising a recognition site for Nt.BstNBI, as shown in SEQ ID NO: 643, Nt.BstNBI nicking endonuclease, Bst DNA polymerase (large fragment), Phusion polymerase, and dNTPs. This preferred reaction mixture is designed to allow the polymerase cycling assembly reaction to take place immediately following the strand-displacement amplification without the need for buffer change between the two reactions. As an illustrative and non-limiting example, a combined amplification and assembly reaction mixture can comprise 0.4 mM dNTPs, 0.2 mg/ml bovine serum albumin (BSA), Nt.BstNBI, Bst large fragment, and Phusion polymerase in optimized Thermopol II buffer which consists of 20 mM Tris-HCl, 10 mM (NH₄)₂SO₄, 10 mM KCl, 2 mM MgSO₄, and 0.1% Triton X-100, pH 8.8 at 25° C.

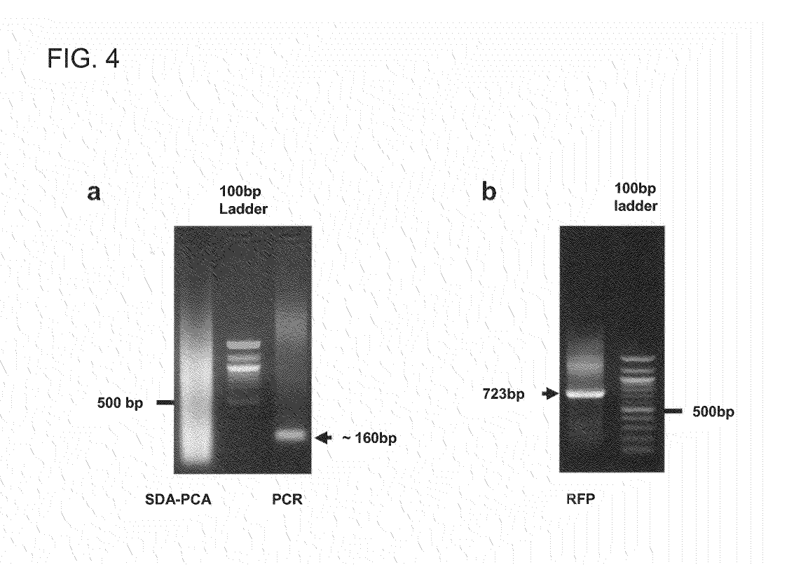

According to embodiments of the present invention, a method of synthesizing a nucleic acid molecule can further comprise amplifying the nucleic acid molecule by a polymerase chain reaction (PCR) amplification to obtain an amplified nucleic acid molecule using a pair of primers matching both ends of the nucleic acid molecule (see FIGS. 4A and 4B). Thus, after completion of the strand-displacement amplification and polymerase cycling assembly reaction in a chamber of a substrate, an aliquot of the reaction can be removed from the chamber of the substrate and be used for PCR amplification. The PCR reaction amplifies the target sequence away from all the shorter incomplete fragments from the PCA.

A PCR amplification reaction of the assembled nucleic acid molecule comprises a DNA polymerase, dNTPs, and a pair of primers complementary to the ends of the nucleic acid molecule. Non-limiting examples of DNA polymerases that can be used for PCR amplification include Phusion polymerase, Taq polymerase, and Pfu DNA polymerase, etc. Preferably, a high-fidelity DNA polymerase is used for the PCR amplification, such as, for example, Phusion polymerase. The reaction conditions for the PCR amplification of the nucleic acid molecules, such as temperature, time, and additional buffer components, can be the same as those used for the polymerase cyclase assembly reaction. The PCR amplification products can be identified and purified using art-recognized techniques, such as, for example, agarose gel electrophoresis.

A nucleic acid molecule obtained by a method of the present invention can also be transformed in a host cell. For example, a nucleic acid molecule comprising a coding sequence for a protein sequence can be cloned into a construct which can subsequently be introduced into a host cell for expression and purification of the encoded protein. Methods for introducing a nucleic acid molecule into a construct, and methods for transforming such constructs into a host cell are well known to those of ordinary skill in the art.

Method of Correcting a Sequence Error

The present invention also provides a method of effecting enzymatic error correction of a nucleic acid sequence, such as synthetic gene sequences, and a method for screening a library of codon variants to obtain a nucleic acid sequence for optimized protein expression.

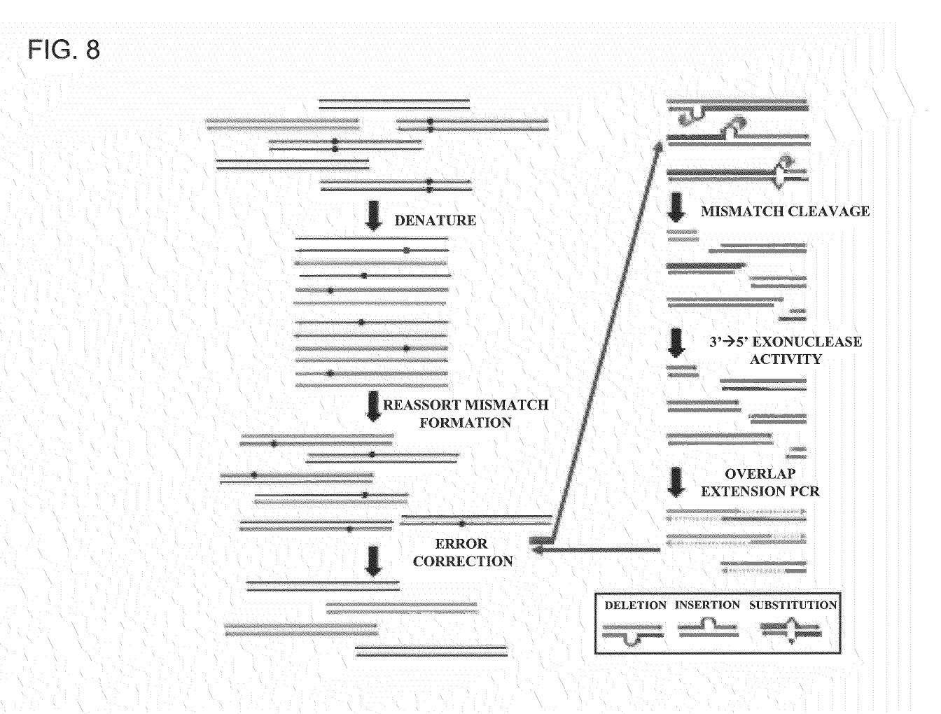

In another general aspect, the present invention relates to a method of effecting enzymatic error correction in a nucleic acid molecule obtained according to a method of the present invention (FIG. 8). According to embodiments of the present invention, a method for correcting a sequence error in a nucleic acid molecule utilizes a mismatch-specific endonuclease, preferably a CEL endonuclease, such as a CEL II endonuclease from celery.

As used herein, a “CEL endonuclease” refers to a member of a family of DNA mismatch-specific endonucleases originally isolated from plant, which is an ortholog of S1 nuclease, prefers double-stranded mismatched DNA substrates, and can cut a mismatch site in a heteroduplex efficiently at neutral pH. CEL endonucleases have been isolated from various plants, such as celery (Yang et al, Biochemistry 39:3533-3541 (2000), Oleykowski et al, Nucleic Acids Res. 26:4597-4602 (1998)) and spinach (Pimkin et al., BMC Biotechnology 2007, 7:29). CEL endonuclease is not inhibited by high GC content, and can cut mismatch-containing heteroduplexes efficiently whether the mismatches are base substitutions, insertions or deletions anywhere from 1 to at least 12 nucleotides. CEL endonuclease is able to act efficiently on molecules with multiple mismatches, even with only five nucleotides between mismatches. Additionally, it can handle substrates anywhere from 40 bp to approximately 30 kb. Its broad substrate specificity and low non-specific activity have made CEL nuclease one of the best tools for mismatch detection (Yang et al, Biochemistry 39:3533-3541 (2000), Oleykowski et al, Nucleic Acids Res. 26:4597-4602 (1998), Kulinski et al, Biotechniques 29:44 (2000), Yeung et al, Biotechniques 38:749-758 (2005), Qiu et al, Biotechniques 36:702-707 (2004)).

As used herein, the term “CEL II endonuclease from celery” refers to a CEL endonuclease originally isolated from celery. It has cleaves with high specificity at the 3′ side of any mismatch site in both DNA strands, including all base substitutions and insertions/deletions up to at least 12 nucleotides (Qiu et al. Qiu et al, Biotechniques 36:702-707 (2004). A CEL II endonuclease from celery is commercially available as Surveyor® endonuclease from Transgenomic as part of the Surveyor Mutation Detection Kit, but can also be produced/purified using methods known in the art. Other mismatch-specific endonucleases, such as T7 endonuclease I, T4 endonuclease VII and Escherichia coli endonuclease V, can also be used in the present invention.

As used herein, a “sequence error” or “error” refers to any change in the nucleotide sequence of a nucleic acid molecule that is different from the desired target sequence for the nucleic acid molecule. The sequence error can be a substitution, insertion, or deletion of in the sequence. Preferably, the error is a substitution, insertion, or deletion, of 1-12 nucleotides.

As used herein, the term “heteroduplex” refers to a double stranded nucleic acid molecule having a target sequence comprising one or more sequence errors in one strand and a complementary sequence of the target sequence free of the one or more sequence errors in the other strand. The heteroduplex comprises one or more mismatch sites resulting from the one or more sequence errors.

According to embodiments of the present invention, a heteroduplex can contain multiple mismatch sites, all of which can be corrected simultaneously by a method for correcting a sequence error as described herein. According to embodiments of the present invention, a mismatch site on the heteroduplex can comprise a substitution, a deletion, or an insertion. The mismatch can comprise anywhere from 1 to at least 12 nucleotides, such as a mismatch of at least 1, 2, 3, 4, 5, 6, 7, 8, 9, 10, 11, or 12 nucleotides.

According to embodiments of the present invention, a method for correcting a sequence error is performed on a nucleic acid molecule obtained by a method of synthesizing a nucleic acid molecule according to an embodiment of the present invention. Nucleic acid molecules are first heat denatured and then cooled to allow for reannealing to obtain heteroduplexes comprising one or more mismatch sites resulting from a sequence error during oligonucleotide synthesis, amplification, and/or assembly. Denaturing and reannealing the nucleic acid molecules allows for the pairing of a strand containing a sequence error with a complementary strand having the correct sequence, creating a heteroduplex in which the mismatch site is exposed (FIG. 8). The nucleic acid molecules are preferably denatured and reannealed in a polymerase buffer, such as 1× Taq buffer or 1× Phusion buffer (available from New England Biolabs).

A typical denaturation temperature that can be used to denature the nucleic acid molecules is about 95° C., however the denaturation temperature can be varied depending on the specific sequence of the nucleic acid molecule and its melting temperature. After heat denaturation, the denatured nucleic acid molecules are cooled to a temperature of about 25° C., and are preferably slow-cooled, to promote re-annealing and heteroduplex formation. For example, denatured nucleic acid molecules can be reannealed by slow cooling from a denaturation temperature of 95° C. by first cooling to a temperature of 85° C. at a rate of 2° C./sec and holding at 85° C. for 1 min, followed by cooling to 25° C. at a rate of 0.3° C./sec, holding for 1 min at every 10° C. interval. As another illustrative example, denatured nucleic acid molecules can be reannealed after heat treatment by first slow cooling to a temperature of 85° C. at a rate of 2° C./sec, followed by cooling to 25° C. at 0.1° C./sec.

According to embodiments of the present invention, the obtained heteroduplexes are then treated with a mismatch-specific endonuclease, such as a CEL endonuclease, under conditions that that allow for the endonuclease to cleave the heteroduplex at the site of the mismatch (FIG. 8, right panel). The CEL endonuclease, such as a CEL II endonuclease from celery, cuts each strand at the 3′ side of the mismatch site, creating fragments of the heteroduplex, wherein each fragment has a 3′ overhang comprising the mismatch site (Yeung et al., Biotechniques 38:749-758 (2005)). Error-free nucleic acid duplexes remain intact and are not affected by CEL endonuclease treatment. In a preferred embodiment, the CEL endonuclease is a CEL II endonuclease from celery, such as Surveryor® nuclease, obtainable from Transgenomic as part of the Surveyor Mutation Detection Kit.

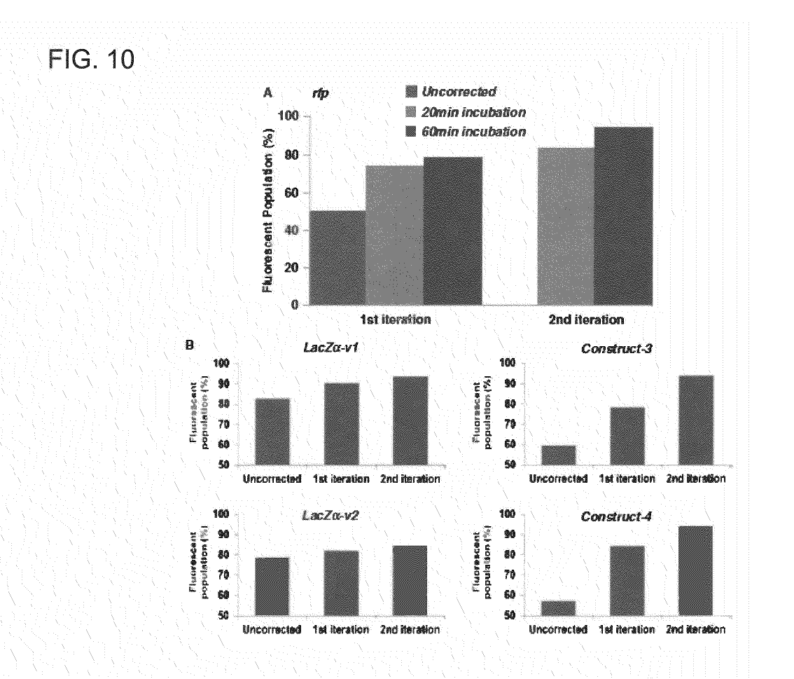

According to embodiments of the present invention, mismatch site recognition and cleavage by a CEL endonuclease can be performed at a temperature of about 25° C. to about 42° C., and for an incubation period of between 20 minutes and 60 minutes, however longer incubation times and higher temperatures give slightly higher levels of corrected nucleic acid molecule (see FIG. 10A). Thus, according to a preferred embodiment, mismatch site recognition and cleavage by a CEL endonuclease is performed at 42° C. for 60 minutes.

Following treatment of the heteroduplex with a CEL endonuclease, an overlap extension PCR amplification is performed using a proofreading DNA polymerase to obtain the nucleic acid molecule with the corrected sequence.

As used herein, the term “proofreading DNA polymerase” refers to a DNA polymerase that possesses 3%5′ proofreading and exonuclease activity of nucleic acid duplexes, such that the DNA polymerase can remove the 3′ overhang comprising a mismatch site of a nucleic acid molecule. Examples of proofreading DNA polymerases that can be used in a method of the present invention include, but are not limited to Phusion polymerase, platinum Taq DNA polymerase High Fidelity (Invitrogen), Pfu DNA polymerase, etc. Preferably, the proofreading DNA polymerase is Phusion polymerase.

As used herein, the term “overlap extension polymerase chain reaction” or “OE-PCR” refers to an in vitro technique to join together two or more nucleic acid fragments that contain complementary sequences at the ends. When the fragments are mixed, denatured and reannealed, the strands having the matching complementary sequences at their ends overlap and act as primers for each other. Extension of this overlap by DNA polymerase produces a molecule in which the original sequences are spliced together.

According to embodiments of the present invention, the OE-PCR links and amplifies the cleaved fragments of the heteroduplex into a full-length, mismatch-site free nucleic acid molecule. During overlap-extension PCR, the 3′-->5′ exonuclease activity of the proof-reading DNA polymerase chews away any 3′ overhangs, which can contain mismatched bases, insertions or deletions, produced by cleavage of the mismatch site by the CEL endonuclease. The error-free fragments are extended and amplified into full-length nucleic acid molecules or gene constructs by the proof-reading DNA polymerase. Intact and error-free nucleic acid duplexes can also be amplified by the overlap extension PCR amplification in the presence of a pair of primers encompassing both ends of the nucleic acid molecule.

Appropriate buffer conditions for correcting a sequence error according to a method of the present invention can be dictated by the proofreading DNA polymerase being used. For example, if Phusion DNA polymerase is used as the proofreading DNA polymerase, synthesized nucleic acid molecules can be diluted in Phusion polymerase reaction buffer. Denaturing and re-annealing the nucleic acid molecules to obtain heteroduplexes can then be performed in the Phusion polymerase reaction buffer, to which the CEL endonuclease can be added for mismatch site recognition and cleavage, followed by addition of the Phusion polymerase for performing overlap extension PCR. A reaction mixture for correcting a sequence error according to a method of the present invention can further comprise an enhancer, such as DNA ligase (Yeung et al. Biotechniques 38:749-758 (2005), Qui et al, Biotechniques 36:702-707 (2004), Quan et al, Nat. Biotechnol. 29:449-452 (2011)).

The efficiency of a method for correcting a sequence according to an embodiment of the present invention can be affected by the amount of enzyme, and the amount of re-annealed nucleic acid molecules, a portion of which comprise a heteroduplex substrate, in addition to other reaction parameters, including the reaction time, temperature, buffer composition, number of iterations of sequence error correction, etc. According to embodiments of the present invention, the concentration of the re-annealed nucleic acid molecule in the reaction is about 40 ng/μL to about 50 ng/μL, and the concentration of the CEL endonuclease, is about 2.5-10 ng/μl of SURVEYOR® Nuclease (Transgenomics).

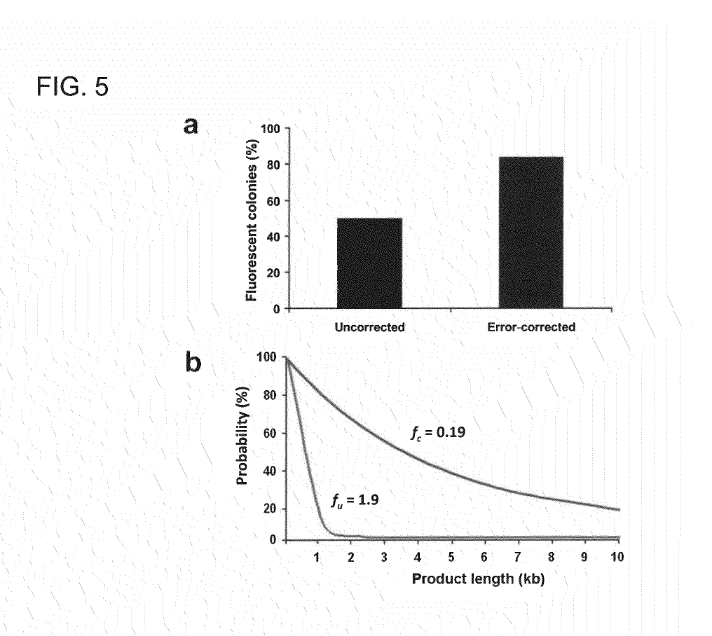

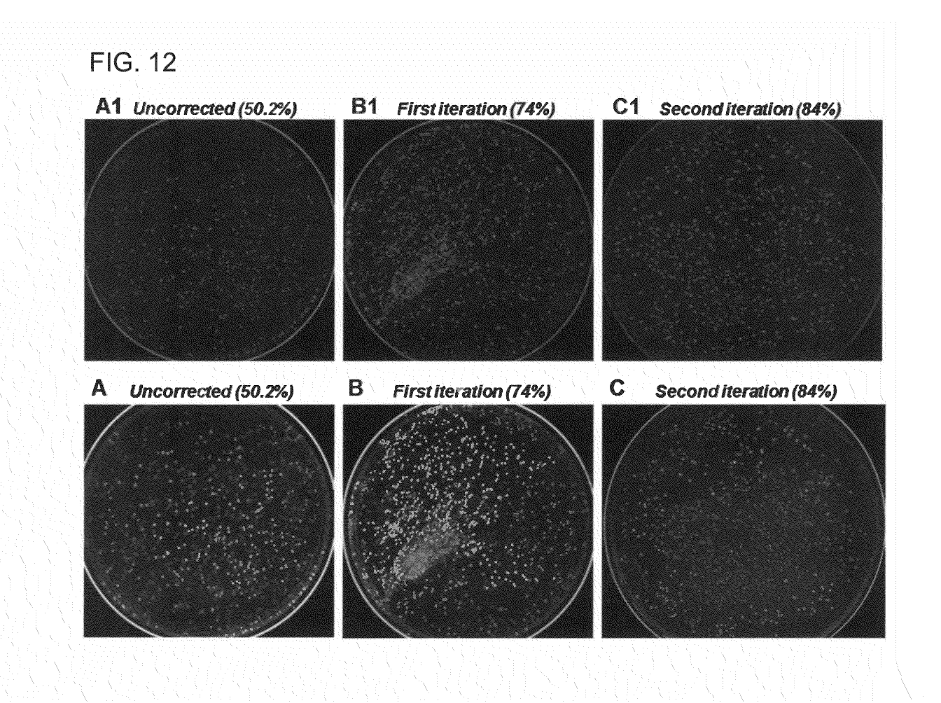

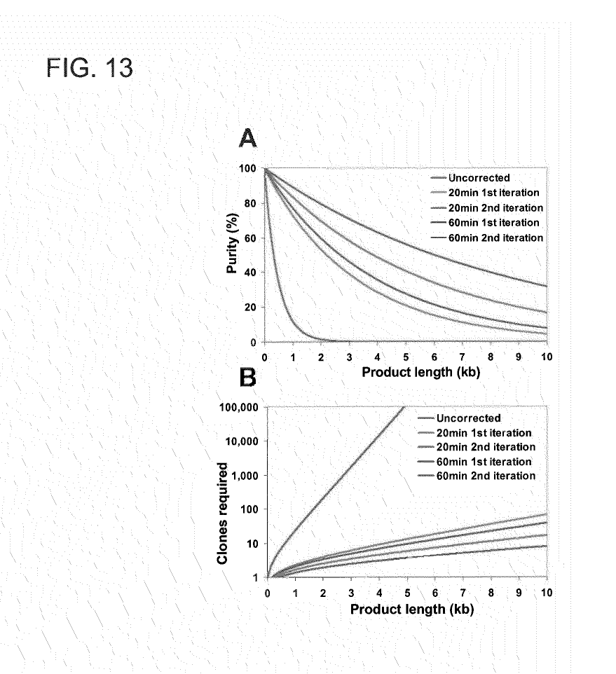

According to embodiments of the present invention, a single round of sequence error correction can be performed, or multiple rounds of error correction can be performed, such as, for example two rounds of error correction. When two rounds of error correction are performed, the nucleic acid molecules obtained from the overlap extension PCR amplification from the first round are diluted to an appropriate concentration in the appropriate reaction buffer. Mismatch recognition and cleavage by the CEL endonuclease, followed by overlap extension PCR can then be performed as described above. Multiple iterations of sequence error correction can increase the probability of correction sequence errors and obtaining an error-free population of a synthesized nucleic acid molecule (see FIGS. 10A, 10B, and 12). The optimal number of iterations of sequence error correction can depend on the length of the synthesized nucleic acid molecule. For example, shorter nucleic acid molecules may require only one or two iterations of sequence error correction, whereas longer nucleic acid molecules may require more than two iterations of sequence error correction to obtain an error free population of nucleic acid molecules (see FIGS. 5B, 13A and 13B).

According to embodiments of the present invention, sequence error correction provides a synthesized nucleic acid molecule, or synthesized gene, with a higher probability of having the correct sequence, as compared to the synthesized nucleic acid molecule that is obtained without performing sequence error correction (FIG. 5A, 13A, 13B).

The corrected, amplified nucleic acid molecules that are obtained from overlap extension PCR amplification after CEL endonuclease treatment can be transformed into a host cell. Conventional techniques well known to one of ordinary skill in the art for transforming nucleic acid molecules of interest into a host call can be used. The corrected, amplified nucleic acid molecules can also be operably linked to a reporter gene sequence to determine the efficiency of error correction (FIG. 12).

In a particularly preferred embodiment, the present invention provides a method of on-chip gene synthesis for obtaining at least one synthesized gene in combination with sequence error correction of the synthesized gene to increase the probability of obtaining the synthesized gene with the correct sequence. The method is carried out on a microchip comprising a plurality of chambers. Each chamber comprises a plurality of oligonucleotides immobilized to the surface microchip, and each oligonucleotide comprises a universal adaptor and a portion of the gene sequence. The plurality of oligonucleotides in the same chamber comprise overlapping portions of the gene sequence to be synthesized. The plurality of oligonucleotides in each chamber are amplified by strand displacement amplification in a reaction mixture comprising a primer, a strand displacement polymerase and a nicking endonuclease. The amplified oligonucleotides are then assembled within the same chamber without the need for any buffer change by a polymerase cycling assembly reaction to obtain at least one nucleic acid molecule. The nucleic acid molecule can comprise the entire desired gene, or a portion of the desired gene to be synthesized. When the assembled nucleic acid molecule comprises only a portion of the desired gene, the nucleic acid molecules can be amplified by PCR amplification and further assembled into the synthesized gene. Sequence errors in the synthesized gene are then corrected by a method of correcting a sequence error according to an embodiment of the present invention.

Method of Screening a Library of Codon Variants

In yet another general aspect, the present invention relates to a method for screening a library of codon variants to obtain a nucleic acid sequence for optimized protein expression.

As used herein, “a library of codon variants” refers to a collection of nucleic acid molecules having different DNA sequences that all translate to the same amino-acid sequence. According to embodiments of the present invention, a library of codon variants is obtained according to a method for synthesizing a nucleic acid molecule as provided by the present invention. In a preferred embodiment, the library of codon variants is synthesized on a single substrate that is divided into a plurality of chambers, wherein in each chamber, the synthesis of a unique codon variant is carried out.

According to embodiments of the present invention, a library of codon variants can be designed using an unbiased codon usage table, in which codons representing an amino acid are used with equal frequency. For example, the codons TGT and TGC both encode a cysteine residue. Thus, when designing a library of codon variants for a protein comprising a cysteine residue in its amino acid sequence, the codons TGT and TGC can be used with equal frequency at that position.

According to embodiments of the present invention, a library of codon variants is then amplified by PCR amplification to obtain an amplified library of codon variants. Any method known in the art in view of the present disclosure can be used to amplify the library of codon variants.

The amplified library of codon variants can then be operably linked to a reporter gene sequence to obtain a library of reporter gene constructs. As used herein, the terms “link” and “linking” refer to the attachment of two nucleic acid molecules via a covalent bond. For example, linking can be performed enzymatically by a DNA ligase enzyme. As used herein, the term “reporter construct” refers to a double stranded nucleic acid duplex comprising a sequence of a codon variant operably linked to a sequence of a reporter gene that can be introduced into a host cell and subsequently translated by the endogenous translational machinery of the host cell. Preferably, a reporter construct is compatible with the E. coli translational machinery. As used herein, the term “operably linked” refers to the covalent attachment of two nucleic acid molecules encoding protein sequences in-frame, such that when the linked nucleic acid molecules are translated into protein, the translated proteins are of the correct amino acid sequence. Methods are well-known in the art for operably linking two nucleic acid molecules together to create a reporter construct, such as, for example, a circular polymerase extension cloning (CPEC) method (Quan and Tian, PloS ONE 4:e6441 (2009)).

Examples of reporter gene sequences that can be operably linked to a library of reporter constructs include, but are not limited to, sequences encoding fluorescent proteins, such as green fluorescent protein (GFP) and red fluorescent protein (REP), and the lacZa gene. For example, a library of codon variants can be operably linked to the N-terminus of a nucleic acid molecule encoding GFP by cloning the library of codon variants into a pAcGFP expression vector by CPEC.

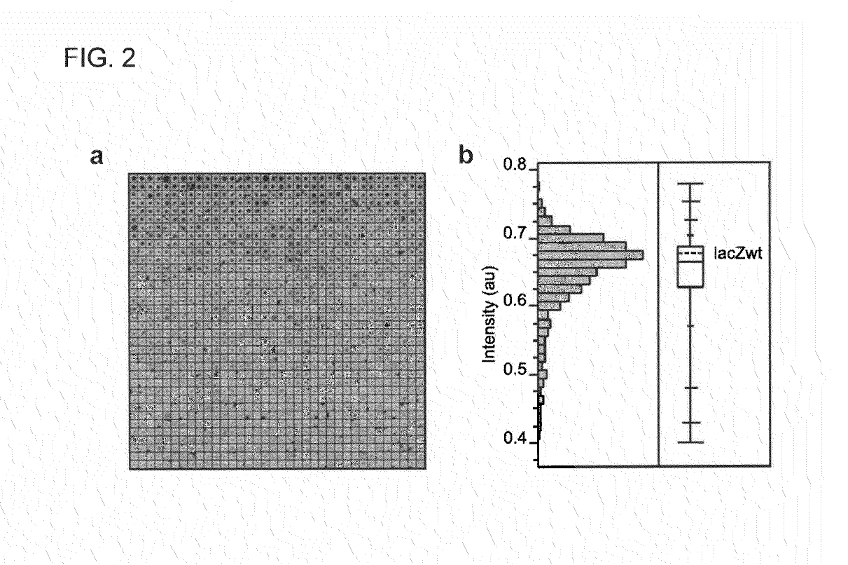

According to embodiments of the present invention, the library of reporter constructs is then introduced into a host cell. In a preferred embodiment, the host cell is E. coli. Once introduced into the host cell, the expression level of each codon variant can be determined by measuring the level of expression of the reporter gene as it is translated by the endogenous translation machinery of the host cell. The method used to measure protein expression of the reporter gene will depend upon the reporter gene. For example, when lacZa is used as the reporter construct, the transformed host cells can be grown in the presence of isopropyl-D-thiogalactopyranoside (IPTG), which is cleaved by the lacZa protein, turning the cells a blue color (FIGS. 2A and 2B). The intensity of the blue color can be quantitatively determined by, for example, measuring the absorbance, to determine the level of protein expression of the reporter gene. The level of expression of the reporter gene is directly correlated to the level of expression of the codon variant to which it is operably linked, thus a high level of reporter gene expression indicates a high level of protein expression of the codon variant.

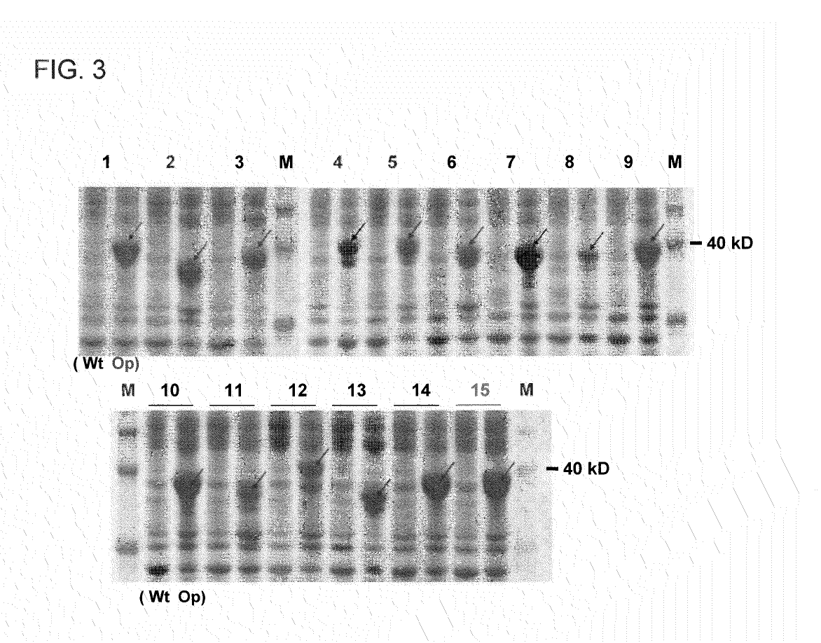

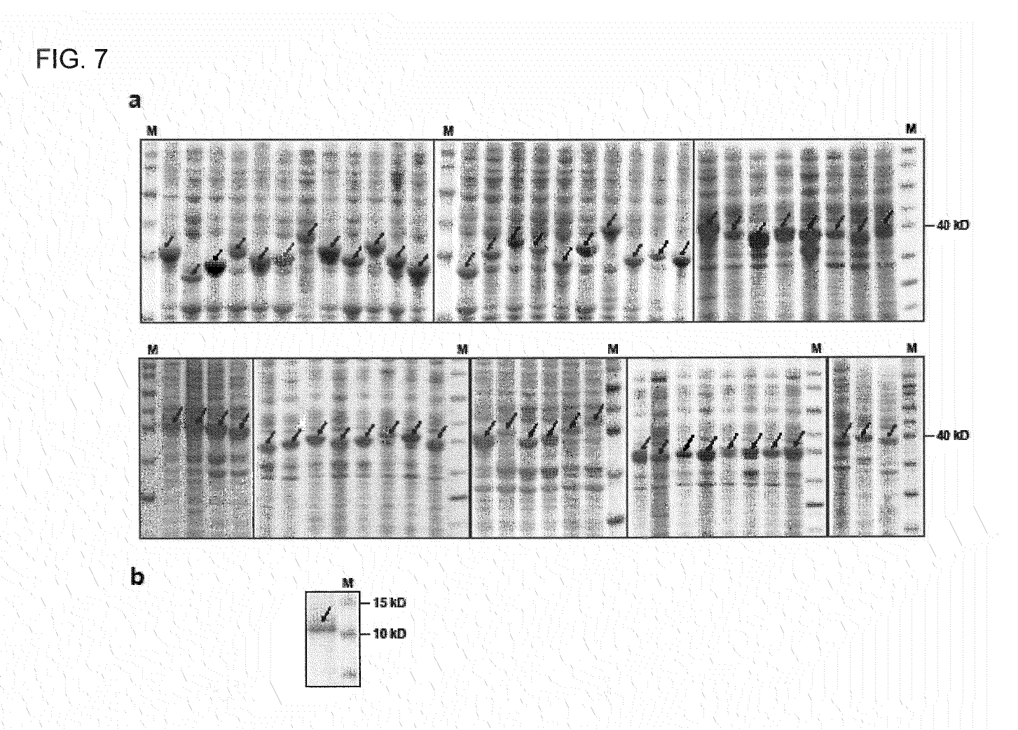

As another illustrative example, when the sequence of a fluorescent protein is operably linked to a library of codon variants as the reporter gene sequence, expression of the reporter gene sequence can determined using fluorescence techniques, such as fluorescence microscopy, to quantitate the level of fluorescence, and therefore the level of protein expression, of the fluorescent protein. Although not as high-throughput, conventional methods of measuring protein expression can be used, such as growing cells under conditions that promote protein expression, and evaluating the level of protein expression by analyzing the cell protein extract on an SDS-PAGE gel (FIGS. 3, 7A and 7B).

Conditions for growing the host cell in a method for screening a library of codon variants according to an embodiment of the present invention will depend upon the species of the host cell. One skilled in the art will be able to readily determine the appropriate growth conditions, including growth media, incubation temperature, time, etc. For example, if the host cell is E. coli, appropriate growth conditions include liquid culture in Luriana Broth (LB) media, or growth on solid LB agar media, at a temperature of 37° C.

According to embodiments of the present invention, the nucleic acid sequence optimized for protein expression, as determined by the level of protein expression of the reporter construct, can be determined by isolating and sequencing the identified nucleic acid molecule by art recognized techniques for purifying and sequencing nucleic acid molecules from cells.

According to embodiments of the present invention, a method for screening a library of codon variants to obtain a nucleic acid sequence for optimized protein expression can be used to identify a codon variant with either high, low, or intermediate protein expression.

In yet another general aspect, the present invention provides a kit for performing on chip gene synthesis. The kit comprises a universal primer having a recognition site for a nicking endonuclease, the nicking endonuclease, a strand displacement DNA polymerase, a high fidelity DNA polymerase, and a mismatch specific endonuclease.

A kit according to an embodiment of the present invention can be used to synthesize genes on chip, and to correct any sequence errors in the synthesized genes introduced during synthesis and assembly.

In a preferred embodiment, a kit according to an embodiment of the present invention comprises a universal primer having a recognition site for Nt.BstNBI, Nt.BstNBI as the nicking endonuclease, Bst large fragment as the strand displacement polymerase, Phusion polymerase as the high fidelity DNA polymerase, and Surveyor nuclease as the mismatch specific endonuclease.

The following examples are to further illustrate the nature of the invention. It should be understood that the following examples do not limit the invention and that the scope of the invention is determined by the appended claims.

### EXAMPLES

The following abbreviations will be used in the Examples, unless stated otherwise:

Oligonucleotide (oligo)

Polymerase chain reaction (PCR)

Nicked strand-displacement amplification (nSDA)

Polymerase cycling assembly (PCA)

Green fluorescent protein (GFP)

Red fluorescent protein (RFP)

Transcription factor (TF)

Enzymatic error correction (ECR)

Overlap-extension PCR (OE-PCR)

### Example 1

### Synthesis of a Nucleic Acid Molecule on-Chip, Enzymatic Error Correction, and Screening a Library of Codon Variants

Oligonucleotide Synthesis on Cyclic Olefin Polymer (COC) Chips.

Oligonucleotide synthesis, amplification and assembly were performed on the same chip in an effort to achieve additional increases in the throughput of nucleic acid molecule synthesis. Chip oligos were synthesized using a custom-made inkjet DNA microarray synthesizer on embossed cyclic olefin copolymer (COC) chips (Ma et al, J. Mater. Chern. 19:7914-7920 (2009); Saaem et al, ACS Applied Materials and Interface 2:491-497 (2010)). Gene construction oligos were designed to be 48 or 60 bases long with a 25-base universal adaptor sequence at the 3′ end, which provided a nicking site and anchored the oligonucleotide to the surface of the COC chip. The oligonucleotide sequences synthesized comprised a portion of a gene sequence of either the LacZa gene (SEQ ID NOS: 1-12), red fluorescent protein gene (SEQ ID NOS: 13-30), or a Drosophilia transcription factor gene (SEQ ID NOS: 31-642). In the current designs, COC chips were partitioned to form 8 or 30 subarrays of silica thin-film spots 150-μm in diameter and 300-μm in interfeature spacing (center to center). Each chamber, or subarray, in the 30-chamber design could print 361 spots and was used to synthesize only one gene, or gene library up to 0.5-1 kb in length. Multiple spots were used to synthesize one oligonucleotide sequence.

Combined nSDA-PCA Reaction for on-Chip Oligo Amplification and Gene Assembly.

The chambers on the printed COC slides were filled with the nSDA-PCA reaction cocktail containing 0.4 mM dNTP, 0.2 mg/ml bovine serum albumin (BSA), Nt.BstNBI, Bst large fragment, and Phusion polymerase in optimized Thermopol II buffer. The slides with sealed chambers were placed on the slide adaptor of a Mastercycler Gradient thermocycler (Eppendorf) and the combined nSDA-PCA reactions were carried out. nSDA involved incubation at 50° C. for 2 h followed by 80° C. for 20 min; the subsequent PCA reaction involved an initial denaturation at 98° C. for 30 s, followed by 40 cycles of denaturation at 98° C. for 7 s, annealing at 60° C. for 60 s, and elongation at 72° c. for 15 s/kb, and finished with an extended elongation step at 72° C. for 5 min.

PCR Amplification of Assembled Nucleic Acid Molecule.

After nSDA-PCA reaction, 1-2 μl of the reaction from each chamber was used for PCR amplification with Phusion polymerase. The PCR reaction involved an initial denaturation at 98° C. for 30 sec, followed by 30 cycles of denaturation at 98° C. for 10 sec, annealing at 60° C. for 60 sec, and elongation at 72° C. for 15 sec/kb, and finished with a final elongation at 72° C. for 5 min.

Enzymatic Error Correction.

Chip-synthesized genes were diluted in 1× Taq buffer, and were denatured and reannealed by incubating at 95° C. for 2 min before cooling down first to 85° C. at a rate of 2° C. per second and then to 25° C. at a rate of 0.1° C. per second. The reaction (4 μl) was mixed with 1 μl of the Surveyor nuclease reagents (Transgenomic) and incubated at 42° C. for 20 min. The product (2 μl) was PCR amplified, cloned and sequenced.

Image Analysis of E. coli Colonies.

150-mm LB agar plates were spread evenly with transformed E. coli cells and incubated overnight at 37° C. Raw images were acquired by scanning the plates with a computer-controlled HP Photosmart C7180 Flatbed Scanner. Bacterial colonies were then identified as a set of objects ranging from 2 to 30 pixels in diameter on scanned images. An automatic thresholding method using a mixture of Gaussians was used to identify local maxima (Lamprecht et al, Biotechniques 42:71-75 (2007)). The images were converted to grayscale and pixel intensities were inverted. From the set of pixels located in each colony, ten pixels with the maximum intensities were selected and averaged to give an estimate of colony color intensity.

Plasmid Library Construction Using Circular Polymerase Extension Cloning (CPEC) Method.

The commercial vector pAcGFP1 was modified by inserting a His6-tag immediately after the start codon and a TVMV cleavage site (ETVRFQS) in front of the GFP gene. The modified vector was linearized by PCR to add overlapping end sequences with the insert. Transcription factor open reading frames were cloned into the vector using the CPEC cloning method (Quan and Tian, PLoS ONE 4:e6441 (2009), Quan and Tian, Nat. Protoc. 6:242-251 (2011)). Briefly, 250 ng of the linear vector was mixed with inserts at 1:2 molar ratio in a 25 μl CPEC reaction using Phusion polymerase. The reaction involved ten cycles of denaturation at 98° C. for 10 s, annealing at 55° C. for 30 s and extension at 72° C. for 15 s, and finished with an extended elongation step at 72° C. for 5 min. 4 μl of the cloning product was used for direct transformation of E. coli.

Protein Expression Screen.

E. coli libraries of codon variants were cultured on LB agar plates containing 100 μg/ml carbenicillin. From each plate, which had about 1,000-1,500 colonies, 1-10 colonies with the highest GFP signals were selected and cultured overnight in Luria Broth at 37° C. with shaking. The saturated culture was diluted 1:50 in the same media and grown at 37° C. until mid-log phase (A6oo=0.5) when the temperature was shifted to 30° C. and 1 mM final concentration of isopropyl-β-D1-thiogalactopyranodise (IPTG) was added. After another 4 h, 10 ml of each culture was centrifuged and the cell pellet was resuspended in 1× NuPAGE LDS Sample Buffer (Invitrogen). After the samples were heated at 90° C. for 5 min and centrifuged at 14,000 g for 10 min, aliquots of the supernatant were analyzed by SDS-PAGE using a NuPage 4-12% gradient gel (Invitrogen) and stained with EZBlue Gel Staining Reagent (Sigma).

Cleavage and Purification of Transcription Factor-GFP Fusion Proteins.

For intracellular processing of transcription factor-GFP fusion proteins, E. coli cells co-transformed with an optimized transcription factor-GFP plasmid and the pRK1037 vector containing the TVMV protease gene were grown in 2 ml of Luria Broth with 100 μg/ml carbenicillin and 30 μg/ml kanamycin at 37° C. overnight. The saturated culture (1 ml) was added into 500 ml of the same medium and grown at 37° C. to mid-log phase (A6oo=0.5), when the temperature was shifted to 30° C. and IPTG was added to a final concentration of 1 mM. After another 4 h, the cells were harvested by centrifugation.

To purify His6-tagged transcription factor proteins, the cell paste was resuspended in 1×LEW Buffer (USB) and lysed by mixing with 1 mg/ml lysozyme for 30 min followed by sonication. The cell lysate was centrifuged at 10,000 g for 30 min at 4° C. to pellet the insoluble material. The supernatant was transferred to a clean tube for loading on PrepEase Ni-IDA column (USB) under native condition. The insoluble material was resuspended in 1×LEW Buffer and centrifuged at 10,000 g for 30 min at 4° C. The cell pellet was then resuspended in 1×LEW denaturing buffer (USB) and kept on ice for 1 h with occasional stirring to dissolve the inclusion bodies. The suspension was then centrifuged at 10,000 g for 30 min at 4° C. to remove any remaining insoluble material. The supernatant was transferred to a clean tube for loading on PrepEase Ni-IDA column (USB) under denaturing condition following kit instructions.

Results

To effectively use all of the oligonucleotides synthesized on a microarray, the whole microarray was divided into subarrays, each containing only the oligos needed to assemble a longer DNA molecule of about 0.5-1 kb in total length. Subarrays were physically isolated from the rest of the chip by being located in individual wells, eliminating the need for post-synthesis partitioning of the oligo pool. Oligonucleotides were synthesized on an embossed plastic microchip using a custom-made inkjet DNA microchip synthesizer (Saaem et al, ACS Applied Materials and Interface 2:491-497 (2010)). The printing area in each subarray was patterned with 150-μm spots of silica thin film to reduce ‘edge-effects’, which could lead to poor oligonucleotide synthesis (Ma et al, J. Mater. Chern. 19:7914-7920 (2009)). This design allowed a standard 1″×3″ chip surface to be divided into as many as 30 subarrays, each containing 361 silica spots for synthesizing a unique DNA oligonucleotide sequence. With the setup used in this study, 10,830 different 85-mer oligo sequences could be synthesized on a single chip, providing a capacity to produce up to 30 kb of assembled DNA.

An effort was made to achieve additional increases in throughput by integrating oligonucleotide synthesis with amplification and gene assembly on the same chip. In previous work, chemical methods, such as NH₄OH treatment, have been used to cleave oligonucleotides from the chip for subsequent off-chip gene assembly reactions (Tian et al, Nature 432:1050-1054 (2004)). Progress towards automating and miniaturizing these subsequent reactions has been reported using microfluidics, resulting in reduced costs and reagent consumption (Huang et al, Lab Chip 9:276-285 (2009)). In the present invention, isothermal nicking and a strand displacement amplification reaction (nSDA) are first used to amplify oligonucleotides from the microarray surface, followed by a PCA reaction in the same chamber (FIG. 1). Briefly, 60-mer gene construction oligo sequences are synthesized with a 25-mer universal adaptor added at the 3′ end, which is anchored on the chip surface. This adaptor contains a nicking endonuclease recognition site. After array synthesis, a universal primer having SEQ ID NO: 643 hybridizes to the adaptor and initiates continuous elongation and nicking on the extending strand. This is catalyzed by a combination of a strand-displacing polymerase (e.g., Bst large fragment) and a nicking endonuclease (e.g., Nt.BstNBI). The amplification is linear so as to keep the ratios constant among amplified oligonucleotides. The extent of the amplification is adjusted by controlling the reaction time. It is estimated that a 2 h reaction time results in an approximately four-fold amplification.

To avoid complex microfluidic manipulations that would otherwise be required to collect and purify the amplified oligonucleotides for downstream gene assembly reactions, the gene-assembly reaction cocktail was designed to allow the polymerase cycling assembly reaction to take place immediately after strand-displacement amplification without a buffer change. After appropriate concentrations of the amplified oligos were accumulated after nSDA, the reaction mode was switched from isothermal amplification to thermal cycling, which resulted in assembly of the amplified oligonucleotides into a nucleic acid molecule in the same reaction chamber. The gene products were further amplified off-chip by PCR (FIG. 4) using the following primers: for amplification of synthesized LacZ gene primers having SEQ ID NOS 644-645 were used; for amplification of synthesized Drosophilia transcription factor genes primers having SEQ ID NOS 649-650 were used; and for amplification of synthesized red fluorescent protein genes primers having SEQ ID NOS 646-648 were used. The size range of the combined strand-displacement amplification reaction products is currently set at 0.5-1 kb for overall throughput and assembly efficiency considerations. However, longer sequences can be hierarchically assembled from these 0.5-1 kb building blocks.

To reduce gene synthesis errors, a simple yet effective error-correction method was developed using the plant CEL family of mismatch-specific endonucleases, which have been shown to recognize and cleave all types of mismatches arising from base substitutions or from small insertions or deletions. A commercial source of a subtype of the CEL enzymes was the Surveyor nuclease, which has been used primarily for mutation detection (Qiu et al, Biotechniques 36:702-707 (2004)). To use it for error correction, the synthetic genes were first denatured by heat and reannealed, and then treated with Surveyor nuclease to cleave error-containing heteroduplexes at the mismatch sites. The error-free DNA duplexes remained intact and were amplified by overlap-extension PCR.

To test the effectiveness of this approach, chip-synthesized genes encoding red fluorescent protein (RFP) were cloned into an expression vector with and without Surveyor nuclease treatment. Sequencing and automated fluorescent colony-counting experiments were performed to determine and compare error frequencies. By Sanger sequencing 470 randomly selected clones, error frequencies of 1/526 bp (or 1.9 errors per kb) and 1/5,392 bp (or 0.19 errors per kb) were observed before and after Surveyor nuclease treatment, respectively (see Table 1 below). Automated counting of thousands of colonies showed that 50% and 84% of the RFP colonies were fluorescent in untreated and Surveyor nuclease-treated populations (FIG. 5A). The results of the sequencing and the colony counting experiments correlated well according to statistical analysis (FIG. 5B). Another study reported comparable error frequencies using the commercial ErrASE kit (Kosuri et al, Nat. Biotechnol. 28:1295-1299 (2010)).

TABLE 1
Error frequencies as determined by sequencing in chip-synthesized
RFP genes with and without error correction using Surveyor nuclease. Clones were
randomly selected from each population and sequenced from both directions.

| | | | | | | Error |
|---|---|---|---|---|---|---|
| | | | | Total | Bases | Frequency f |
| | Deletionnns | Insertions | Substitutions | Errors | Sequenced | (per kb) |
| Before | 43 | 4 | 10 | 57 | 29,958 | 1.9 |
| correction | | | | | | |
| Surveyor | 6 | 0 | 48 | 54 | 291,180 | 0.19 |
| correction | | | | | | |

To apply high-throughput gene synthesis to optimize protein expression, a study was made of the distribution of protein expression levels of a large number of synthetic genes that all encode the same protein, called ‘codon variants’. LacZ a was used as an example in this study. Expression of lacZ a makes the host E. coli cells turn blue in the presence of isopropyl- -D-thiogalactopyranoside (IPTG). First, synthetic codon variants were designed using an unbiased codon usage table, in which codons representing an amino acid were used with equal frequency. Then, a library of lacZa codon variants was constructed and the variants transformed into E. coli competent cells. A small fraction of the library was plated on solid agar and the blue color intensity of the individual colonies was measured in real time by automated image analysis. Clones representing a full spectrum of protein translation levels could be readily identified with fine shades of differences in protein expression (FIG. 2A). Notably, a bell-shaped distribution of the maximum protein expression levels of random codon variants growing on the plate was observed (FIG. 2B).

Approximately one-third of the variants showed higher expression levels than wild-type lacZa. The expression level of the wild-type gene was slightly above the median level of all the clones with measurable expressions. Although understanding the causes and implications of this distribution requires further study, the distribution made it possible to estimate the translational potential of the lacZa gene in E. coli, which is indicated by the upper boundary in the quantile box plot (FIG. 2B). These observations suggest the feasibility of an experimental approach to reliably obtain gene sequences with the desired protein expression levels in a given expression system.

Next described is the successful development of such an optimization approach in E. coli, which has been a workhorse for expressing a variety of proteins for research and industrial applications. To allow direct measurement of protein expression levels, each target gene is tagged with a GFP reporter gene. Proteins expressed at higher levels resulted in colonies with brighter fluorescence.

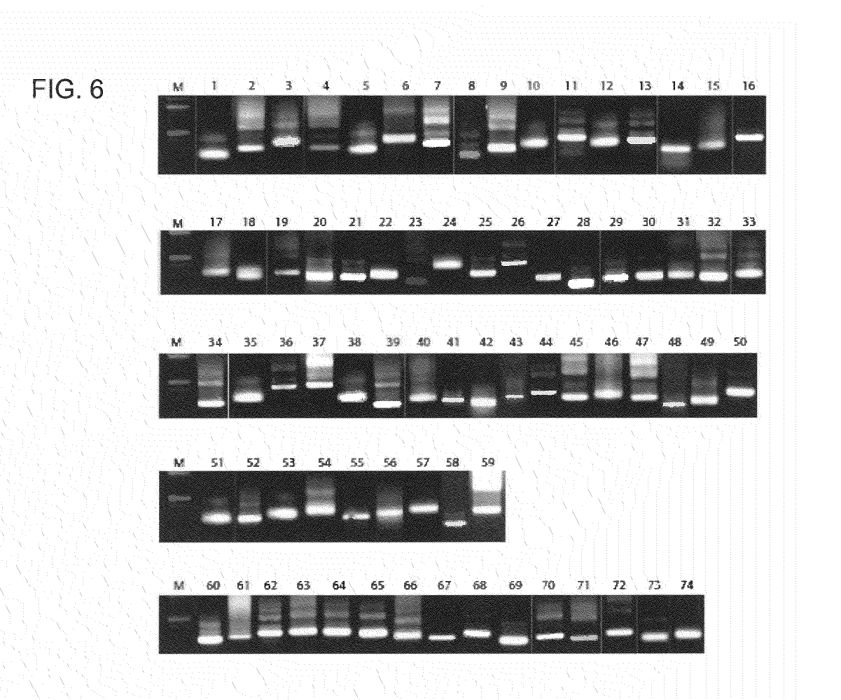

This strategy was applied to optimizing the expression of 74 Drosophila transcription factor protein domains to be used for generating antibodies for the ENCODE (ENCyclopedia Of DNA Elements) Project (The ENCODE Project Consortium, The E.N.C.O.D.E. (ENCyclopedia Of DNA Elements) Project, Science 306:636-640 (2004)). The approach was first tested on 15 candidates that were not expressed in E. coli. Libraries of synthetic codon variants were designed based on an E. coli codon-usage table (Nakamura et al, Nucleic Acids Res. 28:292 (2000)) and constructed using high-throughput gene synthesis technology (FIG. 6). The enzymatic error correction procedure was not performed here because heteroduplexes might form between closely related codon variants. The synthetic genes were fused to the N terminus of GFP and cloned into the pAcGFP expression vector using the sequence-independent circular polymerase extension cloning method (CPEC) (Quan and Tian, PloS ONE 4:e6441 (2009)). E. coli cells were transformed with the plasmid libraries and cultured on agar plates. GFP fluorescence from all colonies was monitored continuously and a small number of highly fluorescent colonies were selected from each pool for sequencing. All colonies contained plasmids with different codon usages throughout the sequence of the candidate proteins.

The sequence-confirmed, highly fluorescent colonies were cultured individually in liquid media and the expression of the protein domains was measured by running the total protein extracts on polyacrylamide gels. High-expression clones were identified for all 15 candidates using this strategy (FIG. 3). In comparison, the wild-type controls cloned into the same vector and cultured under the same conditions showed undetectable protein expression. This result indicates that this method has the capability to reliably increase protein expression from an undetectable level to as high as representing 50-60% of the total cell protein mass.

Encouraged by the high success rate, the same experimental codon optimization procedure was performed for the remaining 59 proteins. Sequencing and protein gel results confirmed that it was possible to predictably obtain high-expression clones for all candidates tested (FIGS. 6 and 7A). Calculation of codon adaptation index (CAI) (Sharp and Li, Nucleic Acids Res. 15:1281-1295 (1987)), which measures synonymous codon usage bias, for each sequence indicates that the average index of the selected, highly expressed synthetic sequences is slightly higher (0.756±0.041) than that of the nonexpressing wild-type sequences (0.663±0.047) (Tables 2 and 3), suggesting a certain degree of correlation between CAI and protein expression level. The highly expressed transcription factor moiety could be freed from the GFP fusion partner by in vivo cleavage with a co-expressed TVMV protease and purified with an Ni-IDA column (FIG. 7B). Removing the GFP fusion partner is desirable for obtaining unique and pure antigen proteins.

TABLE 2
Comparison of CAl values of 15 expression optimized TF sequences
(CAl-opt) vs. wild-type non-expressing sequences. CAI value was
calculated using CAlcal server at http://genomes.urv.es/CAlcal 2.

| Name | CAI-opt | CAI-wt |
|---|---|---|
| AB1 | 0.706 | 0.583 |
| AB11 | 0.795 | 0.709 |
| AC12 | 0.681 | 0.681 |
| AF4 | 0.833 | 0.635 |
| AG3 | 0.738 | 0.707 |
| AR1 | 0.753 | 0.661 |
| B11 | 0.729 | 0.601 |
| D5 | 0.741 | 0.699 |
| F9 | 0.774 | 0.602 |
| K1 | 0.821 | 0.639 |
| K3 | 0.777 | 0.711 |
| K4 | 0.717 | 0.618 |
| K5 | 0.739 | 0.683 |
| L5 | 0.769 | 0.688 |
| M6 | 0.762 | 0.735 |
| Average | 0.756 | 0.663 |
| SD | 0.041 | 0.047 |

TABLE 3
Comparison of CAI values of the remaining 59 expression
optimized TF sequences (CAI-opt) vs. wild-type sequences.

| Name | CAI-opt | CAI-wt | Name | CAI-opt | CAI-wt |
|---|---|---|---|---|---|
| bcd_d2 | 0.684 | 0.63 | BRC_d2 | 0.82 | 0.679 |
| cad_d1 | 0.766 | 0.651 | E74_d1 | 0.798 | 0.601 |
| hb_d2 | 0.753 | 0.673 | E74_d2 | 0.804 | 0.674 |
| lab_d1 | 0.805 | 0.715 | E93_d1 | 0.738 | 0.695 |
| lab_d2 | 0.782 | 0.643 | E93_d2 | 0.807 | 0.731 |
| pb_d2 | 0.726 | 0.673 | mld_d1 | 0.816 | 0.64 |
| Dfd_d1 | 0.806 | 0.689 | salm/salr_d1 | 0.748 | 0.65 |
| Scr_d2 | 0.74 | 0.598 | salm/salr_d2 | 0.772 | 0.693 |
| Antp_d1 | 0.736 | 0.738 | ac_d1 | 0.785 | 0.593 |
| lid_d2 | 0.793 | 0.618 | ac_d2 | 0.715 | 0.683 |
| lilli_d1 | 0.742 | 0.597 | sc_d1 | 0.803 | 0.589 |
| lilli_d2 | 0.786 | 0.668 | I(1)sc_d1 | 0.817 | 0.601 |
| E75_d1 | 0.797 | 0.698 | I(1)sc_d2 | 0.726 | 0.669 |
| E78_d1 | 0.761 | 0.683 | ase_d1 | 0.78 | 0.578 |
| E78_d2 | 0.793 | 0.625 | Dsx_d1 | 0.751 | 0.637 |
| DHR3_d1 | 0.76 | 0.626 | Dsx_d2 | 0.746 | 0.596 |
| DHR3_d2 | 0.774 | 0.704 | Ovo/Svb_d1 | 0.758 | 0.697 |
| EcR_d1 | 0.743 | 0.555 | Ovo/Svb_d2 | 0.771 | 0.749 |
| EcR_d2 | 0.802 | 0.613 | dFOXO_d2 | 0.703 | 0.631 |
| DHR78_d1 | 0.801 | 0.636 | ey_d1 | 0.736 | 0.639 |
| Dis_d1 | 0.721 | 0.658 | ey_d2 | 0.701 | 0.652 |
| Dis_d2 | 0.692 | 0.548 | toy_d1 | 0.768 | 0.576 |
| ERR_d1 | 0.706 | 0.701 | toy_d2 | 0.693 | 0.636 |
| DHR38_ d2 | 0.8 | 0.699 | Stat92E_d2 | 0.802 | 0.645 |
| ftz-f1_d1 | 0.776 | 0.621 | Rx_d1 | 0.746 | 0.658 |
| DHR39_d1 | 0.771 | 0.614 | hbn_d1 | 0.756 | 0.688 |
| DHR39_d2 | 0.704 | 0.546 | otp_d1 | 0.742 | 0.683 |
| DHR4_d1 | 0.707 | 0.55 | dwg_d1 | 0.799 | 0.659 |
| DHR4_d2 | 0.704 | 0.553 | dwg_d2 | 0.789 | 0.7 |
| BRC_d1 | 0.771 | 0.688 | Average | 0.757 | 0.640 |
| | | | SD | 0.038 | 0.055 |

The integration of oligo synthesis and gene assembly on the same microchip facilitates automation and miniaturization, which leads to cost reduction and increases in throughput. On the current chip, each of the 30 chambers was used to synthesize one gene fragment up to 1 kb in length with a 9× redundancy in oligo usage (9 subarray features were used to synthesize one oligo sequence). The estimated cost of chip-oligonucleotide synthesis for this 30 kb of sequence was <$0.001/bp of final synthesized sequences, which is one-tenth of the lowest reported cost (Kosuri et al, Nat. Biotechnol. 28:1295-1299 (2010)). Including enzymatic processing and error correction, the average cost of integrated gene synthesis on a chip is <$0.005/bp of final synthesized gene sequences with an error frequency of <0.2 error/kb. With multiplexing and more advanced chip design, greater throughput and lower costs are potentially achievable.

Protein expression optimization using high-throughput gene synthesis and screening demonstrates a number of advantages over other codon optimization methods, such as testing one design at a time based on unproven design rules. First, the results above indicate that a synthetic gene sequence with a desired protein expression level can be selected through one round of synthesis and screening with high confidence. To efficiently identify high-expression clones for a target protein in E. coli, it is found that for most of the target gene libraries, screening 1,000-1,500 synthetic codon variants for a target protein seems to be sufficient. The capability to achieve not only the maximum but also intermediate levels of protein expression will be valuable for future synthetic biology applications. Second, the screening-based method does not rely on knowing all the rules of codon usage, which are still not completely known. Incomplete knowledge often leads to wrong predictions using other methods. Third, the screening-based method is faster and cheaper and can be performed on a large scale with high-throughput gene synthesis technology. Unpredictability and repeated trial and error using other methods often leads to substantially increased costs, longer production times and lower throughput. Combining high-throughput on-chip gene synthesis and screening can pave the way for systematic investigation of the molecular mechanisms of protein translation.

### Example 2

### Enzymatic Error Correction

Provided below is a detailed characterization of the molecular mechanism of the Surveyor-based sequence error correction reaction, referred to as enzymatic error correction (ECR), and the development of an optimized ECR protocol which further reduced the error rate down to 1 error in 8,700 base pairs.

To eliminate errors in longer synthetic gene constructs, slow and labor-intensive cloning and sequencing methods are traditionally used. If the error rate is high or the sequence is long, large numbers of clones need to be sequenced in order to identify a correct sequence (Carr et al, Nucl. Acids Res. 32:e162 (2004)). If a perfect clone cannot be isolated, site-directed mutagenesis needs to be used to fix errors identified by sequencing (Heckman and Pease, Nature Protocols 2:924-932 (2007), Rabhi et al, Mol. Biotechnol. 26:27-34 (2004), Xiong et al, Nature Protocols 1:791-797 (2006), Linshiz et al, Mol Syst Biol. 4:191. (2008), Marsic et al, BMC Biotechnology 8:44 (2008)). Multiple rounds of cloning, sequencing, and site-directed mutagenesis can significantly increase the cost and turn-around time for gene synthesis.

In order to increase the chance of finding a correct clone, the overall error frequency in the synthetic gene pool needs to be significantly reduced. Methods of using mismatch-binding proteins (e.g., MutS) to remove error-containing DNA heteroduplexes have been developed (Carr et al, Nucleic Acids Res. 32:e162 (2004), Smith and Modrich, Proc. Natl. Acad. Sci. USA 94:6847-6850 (1997), Binkowski et al, Nucleic Acids Res. 33:e55 (2005)). However, MutS-based methods theoretically do not work well for error-rich sequences, because the correct sequences have to outnumber the erroneous sequences in order to avoid being depleted from the synthetic pool.

In comparison, methods using mismatch-cleaving enzymes show an advantage as these enzymes can cleave the heteroduplexes at the vicinity of the mismatch sites, which allows the mutant bases to be subsequently removed by exonuclease activity present in the reaction mixture. A number of enzymes have been tested, including T7 endonuclease I, T4 endonuclease VII, and Escherichia coli endonuclease V, which showed various effectiveness due to various specificities of the enzymes (Young and Dong, Nucleic Acids Res. 32:e59 (2004), Fuhrmann et al, Nucleic Acids Res. 33(6):e58 (2005), Band and Church, Nat. Methods 5:37-39 (2008)).

CEL endonuclease is a new member of the 51 nucleases isolated from celery and prefers double-stranded mismatched DNA substrates (Yang et al, Biochemistry 39:3533-3541 (2000), Oleykowski et al, Nucleic Acids Res. 26:4597-4602 (1998)). It is not inhibited by high GC content, and can cut mismatch-containing heteroduplexes efficiently at neutral pH whether the mismatches are base substitutions, insertions or deletions anywhere from 1 to 12 nucleotides. CEL endonuclease is able to act efficiently on molecules with multiple mismatches, even with only five nucleotides between mismatches. Additionally, it can handle substrates anywhere from 40 bp to approximately 30 kb. Its broad substrate specificity and low non-specific activity has made CEL nuclease one of the best tools for mismatch detection (Yang et al, Biochemistry 39:3533-3541 (2000), Oleykowski et al, Nucleic Acids Res. 26:4597-4602 (1998), Kulinski et al, Biotechniques 29:44 (2000), Yeung et al, Biotechniques 38:749-758 (2005), Qiu et al, Biotechniques 36:702-707 (2004)). Surveyor nuclease, a commercialized form of the CEL endonuclease, is effective in removing errors during chip-based gene synthesis (Quan et al, Nat. Biotechnol. 29:449-452 (2011)).

Reagents.

Chemicals were purchased either from Sigma-Aldrich or VWR. Enzymes were from New England Biolabs. The Surveyor nuclease was purchased from Transgenomic as part of the Surveyor Mutation Detection Kit. GC5 chemical competent cells were purchased from Invitrogen.

Oligonucleotide Synthesis and on-Chip Gene Assembly.

Oligonucleotides were synthesized on a plastic chip using a custom-made inkjet DNA microarray synthesizer (Saaem et al, ACS Applied Materials & Interfaces 2:491-497 (2010)). Gene-construction oligos were designed to be 60-nucleotides long with overlapping regions of similar melting temperatures (Tm=65±2° C.). The exact oligonucleotides synthesized are those having SEQ ID NOS: 651-668. On-chip oligo amplification and gene assembly using combined nicking strand displacement and polymerase cycle assembly reaction was performed as described with minor modifications (Quan et al, Nat. Biotechnol. 29:449-452 (2011)). Briefly, an 8-well incubation adapter (Sigma-Aldrich) was fitted onto the cyclic olefin polymer chips (COC) so that each well contained a synthesized oligo array. The wells were filled with an strand-displacement amplification and polymerase cycling assembly reaction cocktail composed of 0.4 mM dNTP, 0.2 mg/ml BSA, Nt.Bst NBI, Bst large fragment, and Phusion polymerase in an optimized Thermopol II buffer. The chips with sealed chambers were placed on the in situ slide-adapter of a Mastercycler Gradient thermocycler (Eppendorf) to perform combined strand-displacement amplification and polymerase cycling assembly reactions. Strand-displacement amplification involved incubation at 50° C. for 2 hours followed by 80° C. for 20 min; the polymerase cycling assembly reaction involved an initial denaturation at 98° C. for 30 sec, followed by 40 cycles of denaturation at 98° C. for 7 sec, annealing at 60° C. for 60 sec, and elongation at 72° C. for 15 sec/kb, and finished with an extended elongation step at 72° C. for 5 min.

After the combined strand-displacement amplification and polymerase cycling assembly reactions, 1-2 μl of the reaction from each chamber was used for PCR amplification with Phusion polymerase and end primers RFP-R/F/M (SEQ ID NOS.: 669-671). End primers were employed at a concentration of 0.5 μM. The PCR reaction involved an initial denaturation at 98° C. for 30 sec, followed by 30 cycles of denaturation at 98° C. for 10 sec, annealing at 60° C. for 60 sec, and elongation at 72° C. for 30 sec/kb, and finished with a final elongation at 72° C. for 5 min.

Error Correction Reaction of Assembled Genes.

Once PCR amplification of the on-chip assembled nucleic acid molecule was completed, the gene products were purified by agarose gel electrophoresis and extracted to yield a concentration of >100 ng/μL (measured using a Nanodrop analyzer). These PCR products were then diluted with either 1× Taq buffer or 1× Phusion HF buffer to yield a final concentration of 50 ng/μL. The resulting mixture was then melted by heating at 95° C. for 10 minutes, cooled to 85° C. at 2° C./s and held for 1 min. It was then cooled down to 25° C. at a rate of 0.3° C./s, holding for 1 min at every 10° C. interval.

For ECR using a 20 min Surveyor cleavage incubation, 4 μl (200 ng) of the re-annealed nucleic acid molecule product was mixed with 0.5 μl of Surveyor nuclease and 0.5 μl enhancer (which is known to be DNA ligase in nature and enhances the reaction (Yeung et al, Biotechniques 38:749-758 (2005), Qiu et al, Biotechniques 36:702-707 (2004), Quan et al, Nat. Biotechnol. 29:449-452 (2011)) and incubated at 42° C. for 20 min. 2 μl of the reaction mixture was used for subsequent overlap extension PCR (OE-PCR) using the same reaction conditions as the PCR above. The OE-PCR product was cloned and sequenced to serve as the result from the first iteration of error correction. For the second iteration of error correction, the OE-PCR product band was diluted to 50 ng/μL using 1× Taq buffer and re-annealed as before. Similar to the first iteration, a 5 μL reaction consisting of 4 μL re-annealed product, 0.5 μL of Surveyor nuclease and 0.5 μL enhancer was incubated at 42° C. for 20 min. 2 μL of the product was subjected to overlap extension PCR amplification, cloned and sequenced to serve as the result from the second iteration of error correction.

For ECR using a 60 min Surveyor cleavage incubation, 8 μl of the re-annealed nucleic acid molecule product in 1× Phusion buffer (final DNA concentration of 50 ng/μl) was added to 2 μl of Surveyor nuclease and 1 μl enhancer to yield a total of 11 μl that was then incubated at 42° C. for 60 min. 2 μl of the reaction mixture was then subjected to overlap extension PCR amplification, and the resulting PCR product was cloned and sequenced to serve as the result from the first iteration of error correction. For the second iteration, the product from the first iteration was diluted to 50 ng/μl using 1× Phusion buffer and re-annealed as before. Similar to the first iteration, an 11 μl reaction consisting of 8 μl of re-annealed product, 2 μl of Surveyor nuclease and 1 μl of enhancer was incubated at 42° C. for 60 min. 2 μl of the product was used for overlap extension PCR amplification and the PCR product was cloned and sequenced to serve as the result from the second iteration of error correction.

Cloning, Sequencing, and Functional Analysis of Synthetic Genes.

Synthetic gene products, before or after ECR, were cloned into pAcGFP I vector using circular polymerase extension method (CPEC) (Quan and Tian, Nature Protocols 6:242-251 (2011), Quan and Tian, PLoS One 4:e644I (2009)). Briefly, 250 ng of the linear vector was mixed with the synthetic gene products at 1:2 molar ratios in a 25 μl CPEC reaction using Phusion polymerase. The reaction involved 10 cycles of denaturation at 98° C. for 10 seconds, annealing at 55-60° C. for 30 seconds and extension at 72° C. for 15 seconds, and finished with an extended elongation step at 72° C. for 5 min.

2 μl of the cloning product was transformed into GC5 chemically competent cells (Invitrogen) according to the manufacturer's instructions. Cells were grown on agar plates with 100 μg/ml carbenicillin for approximately 16 hours and then kept at room temperature for 48 hours before being imaged in an AlphaImage gel documentation system. The percentage of fluorescent colonies was automatically determined using CellC program (http://sites.google.com/site/cellcsoftware/download). The results were verified by thresholding the UV images using Adobe Photoshop and counting using ImageJ. Sequence analysis was done by extracting plasmids from randomly selected colonies using a miniprep kit (Qiagen), and sequencing of the plasmids was performed at the Duke University Sequencing Facility.

Results

General Design of the Error Correction Reaction Using Surveyor Nuclease

This study relates to the development of a simple and convenient method to effectively remove errors from synthetic genes. The general strategy of using the Surveyor endonuclease to correct errors in synthetic genes is illustrated in FIG. 8. After gene synthesis, the products are denatured and re-annealed to form mismatch-containing heteroduplexes (left panel). The subsequent error correction reaction (ECR, right panel) involves incubation of the re-annealed product with the Surveyor nuclease, followed by overlap extension PCR (OE-PCR) using a proofreading DNA polymerase. The 3′→5′ exonuclease activity of the DNA polymerase removes 3′ overhangs that contain the mismatch base(s) and allows overlap extension to proceed efficiently.

Mismatch structures formed at the deletion, insertion and substitution sites in the heteroduplexes are recognized by the Surveyor mismatch-specific endonuclease, which cuts each strand at the phosphodiester bond at the 3′ side of the mismatch site (Yeung et al, Biotechniques 38:749-758 (2005)). During the subsequent OE-PCR reaction, the 3′→5′ exonuclease activity of the proof-reading DNA polymerase chews away any 3′ overhangs that contain the mismatch base(s) (substitutions and insertions). Finally, the error-free fragments are extended and amplified into full-length gene constructs by the DNA polymerase.

Determination of Error Frequency of on-Chip Gene Synthesis

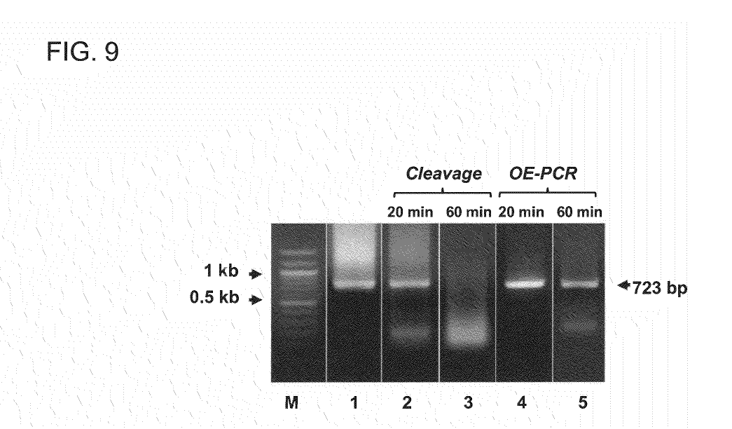

Integrating oligo synthesis with gene assembly on a microchip can significantly reduce synthesis cost and increase throughput. As described above, DNA microarrays were synthesized using a custom inkjet DNA synthesizer and a combined nSDA-PCA reaction was used for on-chip oligo amplification and gene assembly. To determine error frequency of on-chip gene synthesis without error correction, red fluorescent protein (rfp) was chosen as a test gene for convenient screening of functionally correct genes, which served as a good approximation of sequence correct genes. After the combined strand-displacement amplification and polymerase cycling amplification reactions, the 723-bp rfp construct was amplified by PCR (FIG. 9, lane 1) and inserted into a modified pAcGFP1 expression vector using the CPEC cloning method as described above. After transformation into bacteria, the colonies produced were either non-fluorescent, dimly or brightly fluorescent. A rough approximation of synthesis quality without error correction could be made using colony counts on agar plates. Using automated colony counting, it was found that 50.2% of the rfp colonies formed from uncorrected product fluoresced brightly (FIG. 10A).

DNA sequencing was performed on 42 randomly picked rfp colonies from both directions. Random clones of synthetic genes before (Without ECR) or after one or two ECR iterations (ECR1, ECR2) were sequenced in both directions. Surveyor incubation time (20 min or 60 min) was indicated. The occurrence of different type of errors was counted. The results are shown below in Table 4. The sequencing results indicate an error rate of approximately 1.9/kb Deletions were found to be the dominant form of errors (75.4%), which was similar to column DNA synthesis where monomers are not successfully added to all of the growing polymer chains.

TABLE 4
Error analysis of synthetic gene sequences before and after ECR with Surveyor nuclease.

| | | | 20 min | 20 min | 60 min | 60 min |
|---|---|---|---|---|---|---|
| Error Type | | Uncorrected | #1 | #2 | #1 | #2 |
| Deletion | Single-base | 30 | 2 | 0 | 0 | 0 |
| | deletion | | | | | |
| | Multiple-base | 13 | 1 | 0 | 0 | 0 |
| | deletion | | | | | |
| Insertion | Single-base | 3 | 0 | 0 | 0 | 0 |
| | insertion | | | | | |
| | Multiple-base | 1 | 0 | 0 | 0 | 0 |
| | insertion | | | | | |
| Substitution | Transition | | | | | |
| | G/C to A/T | 3 | 2 | 1 | 3 | 2 |
| | A/T to G/C | 3 | 4 | 2 | 0 | 1 |
| | Transversion | | | | | |
| | G/C to C/G | 0 | 0 | 0 | 2 | 0 |
| | G/C to T/A | 1 | 1 | 1 | 4 | 1 |
| | A/T to C/G | 2 | 0 | 0 | 1 | 2 |
| | A/T to T/A | 1 | 0 | 1 | 1 | 0 |

| | | | | | |
|---|---|---|---|---|---|
| Total errors | 57 | 10 | 5 | 11 | 6 |
| Bases | 9958 | 31866 | 27798 | 42714 | 52206 |
| sequenced | | | | | |
| Error | 1.90 | 0.31 | 0.18 | 0.26 | 0.11 |
| frequency | | | | | |
| (errors per | | | | | |
| kb) | | | | | |
| Error | 526 | 3187 | 5560 | 3883 | 8701 |
| frequency | | | | | |
| (bases per | | | | | |
| error) | | | | | |

Error Correction Reaction with Surveyor Nuclease

Surveyor nuclease has typically been used for mutation detection. A strategy was devised of using it for eliminating errors in synthetic genes, as shown in FIG. 8. To determine the optimal reaction conditions of using Surveyor nuclease for error correction, reaction parameters were systematically varied, such as reagent amount, buffer composition, incubation time, temperature, and number of iterations.

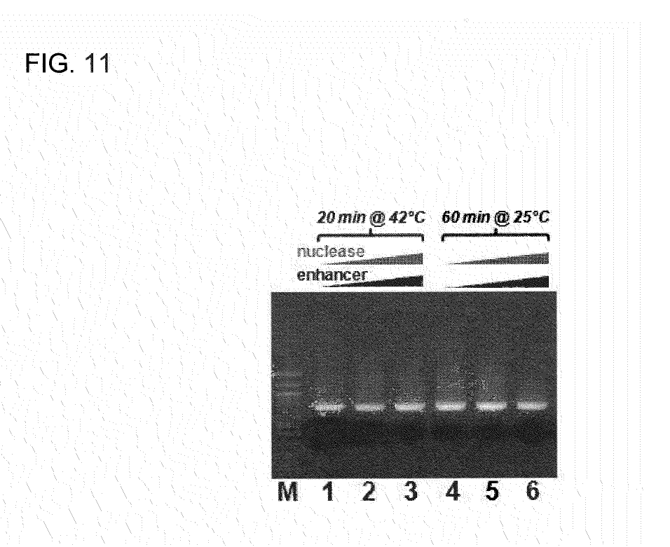

In the first set of experiments, varying amounts of the Surveyor nuclease reagents, including the enzyme and the enhancer were tested. 0.5, 1 and 2 μl of Surveyor nuclease reagents were mixed with 200 ng of re-annealed synthetic rfp product. Incubations were performed either at 42° C. for 20 min or 25° C. for 60 min. After overlap extension PCR amplification, products from all variations were run on an agarose gel (FIG. 11). All bands on the gel appeared to be similar, indicating little difference with increased enzyme concentration.

Depending on the length and sequence quality of the synthetic gene products, after re-annealing and incubation with the Surveyor nuclease, the amount of intact full-length product that can survive the cleavage may be very limited. To assess the extent of cleavage of the on-chip synthesized rfp genes, the re-annealed product was incubated with Surveyor for either 20 or 60 minutes at 42° C. FIG. 9 shows that after 20 min of Surveyor treatment, a fraction of the synthetic genes was cleaved into smaller fragments (lane 2); after 60 min, the majority of the genes were cleaved (lane 3). The results suggested that the cleavage by Surveyor nuclease was relatively efficient. It also suggests that the Surveyor cleavage assay can be used as a quick assessment of the sequence quality of the synthetic products. Following cleavage, overlap extension PCR amplification was able to assemble and extend the fragments back to full-length genes, as shown in FIG. 11 (lanes 4 and 5).

Reduction of Error Frequencies after ECR

Both functional colony counting and DNA sequencing were performed to estimate error frequencies of chip-synthesized genes after ECR with 20-min or 60-min Surveyor treatment. It was reasoned that in one round of ECR, sequences containing errors could form homodimers by chance during annealing and thus escape detection and cleavage. A test was, therefore, made to determine whether an additional round of ECR could eliminate more errors. Two iterations of ECR were performed with both 20 min and 60 min incubations as outlined above. Full-length gene products were cloned and used for functional assays and Sanger sequencing in order to estimate error frequencies.

As shown in FIG. 10A, increasing Surveyor cleavage time and number of iterations led to increases in the number of brightly fluorescent colonies. Using 20-min Surveyor treatment, the fluorescent population increased from 50.2% (untreated) to 74% and 84% in the first and second iteration, respectively. Using 60-min Surveyor treatment resulted in 78.4% and 94% fluorescent colonies after the first and second iteration. Example images showing the fluorescent colonies can be found in FIG. 12.

To investigate the repeatability and robustness of the method, it was applied to the synthesis of four additional gene constructs and its effectiveness was measured using functional or reporter assays. Of the four constructs, two were codon variants of the lacZa gene, the expression of which cause the colony to turn blue in the presence of X-gal. The other two constructs could not be screened by their own functions and, therefore, were fused to the N-terminus of the green fluorescent protein (GFP) (FIG. 10B). Blue or fluorescent colonies indicated that there were no frame shifts or mutations in the gene constructs that could abolish the function or expression of the genes. Therefore, the percentage of positive colonies could be used as an approximate indicator of the quality of the sequences. The results from the four additional constructs showed iterative increase in positive populations after each round of ECR (FIG. 10B). As expected from the model predictions shown in FIG. 13A, the small lacZa genes had a large fluorescent population even before error correction (80% positive) as it had fewer errors to begin with due to their short length (174 bp). In comparison, the longer constructs (#3 & 4) had lower percentages of correct colonies to begin with (500 bp, 55-60% positive) but the effect of ECR was more obvious, reaching >90% positive after two iterations (FIG. 10B).

Results from DNA sequencing analysis of randomly selected colonies correlated with the observations made with the colony counting experiments and revealed more details on the correction efficiency of different types of errors. The results in Table 4 showed that ECR with Surveyor was very efficient in reducing errors arising from deletion and insertion events. Most deletion and insertion type of errors could be eliminated after one round of 60-min treatment or two rounds of 20-min treatment. Surveyor treatment was also effective towards substitutions albeit with reduced efficiency. Substitution types of errors were still present after two rounds of 60-min incubations.

For the purpose of developing the most efficient ECR procedure, data in Table 4 indicated that increasing incubation time from 20 min to 60 min reduced error frequency from 0.31 to 0.26 error/kb (16% reduction); while adding another round reduced error frequency from 0.31 to 0.18 error/kb for 20-min incubations (42% reduction) and from 0.26 to 0.11 error/kb for 60-min incubations (58% reduction). It appeared that adding a second round of ECR was more effective than increasing the Surveyor incubation time with only one round of ECR, although the cumulative effects of more iterations and longer Surveyor incubation was most dramatic.

Following the model predictions of Can et al (Nucleic Acids Res. 32:e162 (2004)) and Furhmann et al (Nucl. Acids Res. 33(6):e58 (2005)), statistical analysis was performed to better understand the implication of the results. As can be seen in FIG. 13A, the percentage of gene synthesis products that yield error-free clones decreases exponentially with the length of the product synthesized. Employing ECR for error correction (1 error in 8701 bp for two iterations of 60-min ECR, blue line) significantly increases the probability of locating an error-free clone than without error correction (1 error in 526 bp, red line). This means that dramatically fewer clones need to be sequenced (FIG. 13B). For example, as predicated in FIG. 13B, with ECR one will have to screen, on average, only 8-10 clones of a 10 kb treated or a single 1 kb clone in order to locate a correct one. The model prediction correlated well with the sequencing analysis results.

Analyzing sequencing data of 77 random colonies from the second iteration of the 60-min ECR, 72 of the colonies were found to contain the correct rfp gene. The determined error rate of 0.11/kb meant a >16-fold reduction of errors present in the synthetic pool. With such an improvement, larger DNA targets can be conveniently synthesized and corrected within 2-3 hours without resorting to additional cloning or excessive sequencing.

In conclusion, the method described above performs enzymatic error correction on synthetic genes using Surveyor nuclease, which has the broadest substrate specificity towards all types of mismatches as compared to other known mismatch-specific binding proteins or endonucleases. The method utilizes the mismatch-specific endonuclease activity of the Surveyor enzyme to cut heteroduplex sequences at the mismatch sites and uses the exonuclease activity of the proof-reading DNA polymerase to remove the mismatch bases, followed by an OE-PCR reaction to reassemble the cleaved fragments into full-length gene constructs. The results demonstrate that the optimized ECR procedure is robust and effective for all error types, especially insertions and deletions, yielding superior results than previous methods. The ECR method is probably more suitable for long and error-rich synthetic products and can be performed in less time than MutS-based procedures which require gel shift assay and DNA extraction from polyacryalamide gels. Additionally, in comparison to the commercial ErrASE kit (Kosuri et al, Nat. Biotech. 28:1295-1299 (2010)), the ECR reaction mitigates the need for tittering and excessive enzyme usage. Using the protocol developed in the current study, two ECR iterations could be completed in less than 5 hours and reduces the error frequency by >16-fold.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method of synthesizing a nucleic acid molecule having a target sequence, comprising:
(1) obtaining a substrate having a chamber comprising a plurality of immobilized oligonucleotides for the synthesis of the target sequence,
(2) adding to the chamber a reaction mixture comprising dNTPs, a primer, a strand-displacing polymerase, a nicking endonuclease, a heat-stable DNA polymerase, and a buffer;
(3) amplifying the plurality of oligonucleotides to obtain free amplified oligonucleotides by a nicking strand displacement amplification reaction in the chamber containing the reaction mixture; and
(4) assembling the free amplified oligonucleotides by a polymerase cycling assembly reaction to obtain the nucleic acid molecule; wherein step (4) is conducted in the chamber without the need for a buffer change after step (3).

2. The method according to claim 1, further comprising amplifying the nucleic acid molecule obtained in step (3) by a polymerase chain reaction (PCR) amplification reaction to obtain an amplified nucleic acid molecule, and purifying the amplified nucleic acid molecule.

3. The method according to claim 1, wherein
each of the plurality of oligonucleotides comprises a universal adaptor sequence at the 3′ end of the oligonucleotide and a portion of the target sequence or a portion of a sequence complementary to the target sequence;
the universal adaptor sequence anchors the oligonucleotide to the substrate surface; and
the primer comprises a universal primer complementary to the universal adaptor sequence, the universal primer comprises a nucleotide sequence that is recognized and cut by the nicking endonuclease.

4. The method according to claim 3, wherein the portion of the target sequence or its complementary sequence comprises about 48 to 150 bases in length; and the universal adaptor sequence comprises about 15-35 bases in length.

5. The method according to claim 3, wherein the universal adaptor comprises a Nt.BstNBI recognition site; and the reaction mixture comprises the universal primer, Nt.BstNBI, Bst DNA polymerase, large fragment, Phusion polymerase and a buffer.

6. The method according to claim 1, wherein a plurality of nucleic acid molecules are synthesized in each of a plurality of chambers, and the plurality of chambers are on a microchip.

7. The method of claim 1, further comprising transforming a cell with the nucleic acid molecule obtained from step (3).

8. The method according to claim 2, further comprising correcting a sequence error in the amplified and purified nucleic acid molecule, comprising:
(1) heating and subsequently cooling a plurality of nucleic acid molecules synthesized according to a method of the present invention, thereby forming one or more heteroduplexes, wherein the heteroduplex comprises one or more mismatch sites resulting from the errors;
(2) contacting the one or more heteroduplexes with a mismatch-specific endonuclease under conditions for effective cleavage of the one or more heteroduplexes at the one or more mismatch sites, thereby obtaining cleaved fragments; and
(3) contacting the cleaved fragments with a DNA polymerase having 3′-5′ exonuclease activity under conditions for an overlap extension polymerase chain reaction amplification, thereby producing a plurality of nucleic acid molecules free of the one or more errors.

9. The method of claim 8, wherein the mismatch-specific endonuclease is a CEL endonuclease.

10. The method of claim 8, wherein the CEL endonuclease is a CEL II endonuclease from celery, and the DNA polymerase is Phusion polymerase.

11. The method of claim 8, further comprising transforming a cell with the nucleic acid molecule obtained from step (3).

12. The method of claim 8, further comprising repeating steps (1) to (3) of claim 8.

13. A method for screening a library of codon variants to obtain a nucleic acid sequence for optimized protein expression, the method comprising:
(1) synthesizing the library of codon variants using the method of claim 1;
(2) amplifying the library by a polymerase chain reaction (PCR) amplification reaction;
(3) operably linking the library of codon variants to a reporter gene sequence to obtain a library of reporter constructs;
(4) introducing the library of reporter constructs into a host cell; and
(5) measuring the expression of the reporter gene sequence from the host cell, thereby identifying the nucleic acid sequence for optimized protein expression.

14. The method according to claim 13, further comprising sequencing the identified nucleic acid sequence.

15. A method of on-chip synthesis of a gene comprises:
(1) obtaining a microchip comprising multiple chambers, each chamber comprising a plurality of immobilized oligonucleotides for the synthesis of a target sequence, wherein the target sequence comprises a fragment of the gene;
(2) adding to each of the chambers a reaction mixture comprising dNTPs, a primer, a strand-displacing polymerase, a nicking endonuclease, a heat-stable DNA polymerase, and a buffer;
(3) amplifying the plurality of oligonucleotides to obtain free amplified oligonucleotides by a nicking strand displacement amplification reaction in each of the chambers containing the reaction mixture;
(4) assembling the free amplified oligonucleotides to obtain the target sequence by a polymerase cycling assembly reaction, wherein step (4) is conducted in each of the chambers without the need for a buffer change after step (3).
(5) amplifying the target sequence from step (4) by a polymerase chain reaction (PCR) in each of the chambers;
(6) assembling the amplified target sequences from all chambers into a synthesized gene sequence; and
(7) correcting a sequence error in the synthesized gene sequence, comprising:
i. forming a heteroduplex comprising the synthesized gene sequence, the heteroduplex comprising one or more mismatch sites resulting from the sequence error;
ii. contacting the heteroduplex with a mismatch-specific endonuclease under conditions such that the heteroduplex is cleaved at the mismatch sites to obtain cleaved fragments of the gene; and
iii. contacting the cleaved fragments with a DNA polymerase having 3′-5′ exonuclease activity under conditions for an overlap extension polymerase chain reaction amplification, thereby producing the gene sequence free of the sequence error.

16. The method according to claim 15, wherein
each of the plurality of oligonucleotides comprises a universal adaptor sequence at the 3′ end of the oligonucleotide and a portion of the target sequence or a portion of a sequence complementary to the target sequence;
the universal adaptor sequence anchors the oligonucleotide to the substrate surface; and
the primer comprises a universal primer complementary to the universal adaptor sequence, the universal primer comprises a nucleotide sequence that is recognized and cut by the nicking endonuclease.

17. A kit for performing on chip gene synthesis, the kit comprising:
(1) a universal primer comprising a nucleotide sequence that is recognized and cut by a nicking endonuclease,
(2) the nicking endonuclease;
(3) a strand displacement DNA polymerase;
(4) a DNA polymerase; and
(5) instructions on using the kit for synthesizing a nucleic acid molecule.

18. The kit of claim 17, wherein the DNA polymerase has 3′-5′ exonuclease activity, and the kit further comprises a mismatch-specific endonuclease and additional instructions on enzymatic correction of sequence errors in the synthesized nucleic acid molecule.

19. The kit of claim 18, wherein the mismatch-specific endonuclease is a CEL endonuclease and the DNA polymerase is Phusion polymerase.


SEQUENCE LISTING (671 sequences)

SEQ ID NO: 1 - DNA, 73 bp, Artificial Sequence
  LacZ-1
mgraaytcngargargcnmgracncatcatcatcaccaccactgagcggcatgactcgaccatccgatttttt

SEQ ID NO: 2 - DNA, 73 bp, Artificial Sequence
  LacZ-2
ckngcytcytcngarttyckccangangcraanggnggrtgngcngcygcatgactcgaccatccgatttttt

SEQ ID NO: 3 - DNA, 73 bp, Artificial Sequence
  LacZ-3
garaayccnggngtnacncarytraaymgrytrgcngcncayccnccngcatgactcgaccatccgatttttt

SEQ ID NO: 4 - DNA, 73 bp, Artificial Sequence
  LacZ-4
gngtnacnccnggrttytcccartcyckyckytgyarnacnacngcyagcatgactcgaccatccgatttttt

SEQ ID NO: 5 - DNA, 73 bp, Artificial Sequence
  LacZ-5
acaggaaacagctatgacnatgathacnytrgcngtngtnytrcarmggcatgactcgaccatccgatttttt

SEQ ID NO: 6 - DNA, 73 bp, Artificial Sequence
  LacZ-6
tcatngtcatagctgtttcctgtgtgaaattaatgggtaacatgatccgcatgactcgaccatccgatttttt

SEQ ID NO: 7 - DNA, 73 bp, Artificial Sequence
  LacZ-7
cgnaayagygargargcncgnacncatcatcatcaccaccactgagcggcatgactcgaccatccgatttttt

SEQ ID NO: 8 - DNA, 73 bp, Artificial Sequence
  LacZ-8
cgngcytcytcrctrttncgccarctngcraanggnggrtgngcngcngcatgactcgaccatccgatttttt

SEQ ID NO: 9 - DNA, 73 bp, Artificial Sequence
  LacZ-9
garaayccnggngtnacncarctnaaycgnctngcngcncayccnccngcatgactcgaccatccgatttttt

SEQ ID NO: 10 - DNA, 73 bp, Artificial Sequence
  LacZ-10
gngtnacnccnggrttytcccartcncgncgytgnagnacnacngcnagcatgactcgaccatccgatttttt

SEQ ID NO: 11 - DNA, 73 bp, Artificial Sequence
  LacZ-11
acaggaaacagctatgacnatgathacnctngcngtngtnctncarcggcatgactcgaccatccgatttttt

SEQ ID NO: 12 - DNA, 73 bp, Artificial Sequence
  LacZ-12
tcatngtcatagctgtttcctgtgtgaaattaattaggttagtaccgggcatgactcgaccatccgatttttt

SEQ ID NO: 13 - DNA, 85 bp, Artificial Sequence
  RFP-f1-1
ttcaaatgggaacgtgttatgaacttcgaagacggtggtgttgttaccgttacccaggacgcatgactcgaccatccgatttttt

SEQ ID NO: 14 - DNA, 85 bp, Artificial Sequence
  RFP-f1-2
ttcataacacgttcccatttgaaaccttccgggaaggacagtttcaggtagtccgggatggcatgactcgaccatccgatttttt

SEQ ID NO: 15 - DNA, 85 bp, Artificial Sequence
  RFP-f1-3
ccagtacggttccaaagcttacgttaaacacccggctgacatcccggactacctgaaactgcatgactcgaccatccgatttttt

SEQ ID NO: 16 - DNA, 85 bp, Artificial Sequence
  RFP-f1-4
cgtaagctttggaaccgtactggaactgcggggacaggatgtcccaagcgaacggcagcggcatgactcgaccatccgatttttt

SEQ ID NO: 17 - DNA, 85 bp, Artificial Sequence
  RFP-f1-5
acgaaggtacccagaccgctaaactgaaagttaccaaaggtggtccgctgccgttcgcttgcatgactcgaccatccgatttttt

SEQ ID NO: 18 - DNA, 85 bp, Artificial Sequence
  RFP-f1-6
gcggtctgggtaccttcgtacggacgaccttcaccttcaccttcgatttcgaactcgtgagcatgactcgaccatccgatttttt

SEQ ID NO: 19 - DNA, 85 bp, Artificial Sequence
  RFP-f1-7
catgcgtttcaaagttcgtatggaaggttccgttaacggtcacgagttcgaaatcgaagggcatgactcgaccatccgatttttt

SEQ ID NO: 20 - DNA, 85 bp, Artificial Sequence
  RFP-f1-8
catacgaactttgaaacgcatgaactctttgataacgtcttcggaggaagccatcatagcgcatgactcgaccatccgatttttt

SEQ ID NO: 21 - DNA, 85 bp, Artificial Sequence
  RFP-f1-9
agcctggatgacgttttcatcaaaatttcacacaggaaacagctatgatggcttcctccggcatgactcgaccatccgatttttt

SEQ ID NO: 22 - DNA, 85 bp, Artificial
  RFP-f2-1
cgggaagttggtaccacgcagtttaactttgtagatgaactcaccgtcttgcagggaggagcatgactcgaccatccgatttttt

SEQ ID NO: 23 - DNA, 85 bp, Artificial Sequence
  RFP-f2-2
cgtggtaccaacttcccgtccgacggtccggttatgcagaaaaaaaccatgggttgggaagcatgactcgaccatccgatttttt

SEQ ID NO: 24 - DNA, 85 bp, Artificial Sequence
  RFP-f2-3
tcagagcaccgtcttccgggtacatacgttcggtggaagcttcccaacccatggttttttgcatgactcgaccatccgatttttt

SEQ ID NO: 25 - DNA, 85 bp, Artificial Sequence
  RFP-f2-4
cggaagacggtgctctgaaaggtgaaatcaaaatgcgtctgaaactgaaagacggtggtcgcatgactcgaccatccgatttttt

SEQ ID NO: 26 - DNA, 85 bp, Artificial Sequence
  RFP-f2-5
tttagccatgtaggtggttttaacttcagcgtcgtagtgaccaccgtctttcagtttcaggcatgactcgaccatccgatttttt

SEQ ID NO: 27 - DNA, 85 bp, Artificial Sequence
  RFP-f2-6
gaagttaaaaccacctacatggctaaaaaaccggttcagctgccgggtgcttacaaaaccgcatgactcgaccatccgatttttt

SEQ ID NO: 28 - DNA, 85 bp, Artificial Sequence
  RFP-f2-7
gtgtagtcttcgttgtgggaggtgatgtccagtttgatgtcggttttgtaagcacccggcgcatgactcgaccatccgatttttt

SEQ ID NO: 29 - DNA, 85 bp, Artificial Sequence
  RFP-f2-8
cctcccacaacgaagactacaccatcgttgaacagtacgaacgtgctgaaggtcgtcactgcatgactcgaccatccgatttttt

SEQ ID NO: 30 - DNA, 85 bp, Artificial Sequence
  RFP-f2-9
aaattgagactggaaacgcacggtttcttaagcaccggtggagtgacgaccttcagcacggcatgactcgaccatccgatttttt

SEQ ID NO: 31 - DNA, 85 bp, Artificial Sequence
  AB1-1
ccrgcsgcsctgcayacsttyctgagyccrgcsctgtgygaaaccgtgcgtttccagtctgcatgactcgaccatccgatttttt

SEQ ID NO: 32 - DNA, 85 bp, Artificial Sequence
  AB1-2
graasgtrtgcagsgcsgcyggyggyggrcarttyggmaccagyggratytgcagrcgytgcatgactcgaccatccgatttttt

SEQ ID NO: 33 - DNA, 85 bp, Artificial Sequence
  AB1-3
ctgccrggygtkccrgcsccrctggcsctgatyaaygarcgyctgcaratyccrctggtkgcatgactcgaccatccgatttttt

SEQ ID NO: 34 - DNA, 85 bp, Artificial Sequence
  AB1-4
agyggsgcyggmacrccyggcagsgcrttyggratsgcratrcgytcyggyttyttytcmgcatgactcgaccatccgatttttt

SEQ ID NO: 35 - DNA, 85 bp, Artificial Sequence
  AB1-5
tygcsgayatyctgggycgyggycaygargargaycgygtkgaraaraarccrgarcgyagcatgactcgaccatccgatttttt

SEQ ID NO: 36 - DNA, 85 bp, Artificial Sequence
  AB1-6
rcccagratrtcsgcratrctraarctyttmacrcgmacyggrctrctyttytgrcgrtcgcatgactcgaccatccgatttttt

SEQ ID NO: 37 - DNA, 85 bp, Artificial Sequence
  AB1-7
ccaccatcatcatcaccgyagyccratyagyctggarcaygaycgycaraaragyagyccgcatgactcgaccatccgatttttt

SEQ ID NO: 38 - DNA, 85 bp, Artificial Sequence
  AB1-8
trcggtgatgatgatggtggtgcatatggggaatcggcagcacgccatgggtgggagcttgcatgactcgaccatccgatttttt

SEQ ID NO: 39 - DNA, 85 bp, Artificial Sequence
  AB11-1
tayggygcscarcarcaygcsgcsgcscaycaycartgygaaaccgtgcgtttccagtctgcatgactcgaccatccgatttttt

SEQ ID NO: 40 - DNA, 85 bp, Artificial Sequence
  AB11-2
crtgytgytgsgcrccrtaratyttrctratrtcraasgcrtarttsgcsgcrcgrtcytgcatgactcgaccatccgatttttt

SEQ ID NO: 41 - DNA, 85 bp, Artificial Sequence
  AB11-3
gayccragygtkctgacsaargcstayttygayagyaaratgtaycargaycgygcsgcsgcatgactcgaccatccgatttttt

SEQ ID NO: 42 - DNA, 85 bp, Artificial Sequence
  AB11-4
sgtcagmacrctyggrtccagrtasgcrctrtasgcrctsgcytgrctrctrttrctcaggcatgactcgaccatccgatttttt

SEQ ID NO: 43 - DNA, 85 bp, Artificial Sequence
  AB11-5
gyggycaratggayaarttygcsctggarcgyagyagytayctgagyaayagyagycarggcatgactcgaccatccgatttttt

SEQ ID NO: 44 - DNA, 85 bp, Artificial Sequence
  AB11-6
raayttrtccatytgrccrctcagsgtsgcytgsgtsgtsgtyggsgtrctsgtytgrccgcatgactcgaccatccgatttttt

SEQ ID NO: 45 - DNA, 85 bp, Artificial Sequence
  AB11-7
tgcaccaccatcatcatcacgcstaycayctgggyagycayctgggycaracsagyacscgcatgactcgaccatccgatttttt

SEQ ID NO: 46 - DNA, 85 bp, Artificial Sequence
  AB11-8
tgatgatgatggtggtgcatatgagagcagtccgtcagcacgatatacaacgatattcatgcatgactcgaccatccgatttttt

SEQ ID NO: 47 - DNA, 85 bp, Artificial Sequence
  AC12-1
ccrgtkcaycaycarcargcsagycayagygcsccrtgygaaaccgtgcgtttccagtctgcatgactcgaccatccgatttttt

SEQ ID NO: 48 - DNA, 85 bp, Artificial Sequence
  AC12-2
gytgrtgrtgmacyggsgcytgrttytgyggytgrctytgsgccatrccytcrtarccrcgcatgactcgaccatccgatttttt

SEQ ID NO: 49 - DNA, 85 bp, Artificial Sequence
  AC12-3
cscarccratycaragyggytayctgcaytayggyaaytayggyggytaygarggyatgggcatgactcgaccatccgatttttt

SEQ ID NO: 50 - DNA, 85 bp, Artificial Sequence
  AC12-4
crctytgratyggytgsgcrccrtasgcrccrctsgtsgtytgytgraayggmacsgccagcatgactcgaccatccgatttttt

SEQ ID NO: 51 - DNA, 85 bp, Artificial Sequence
  AC12-5
carctgccrtayccrccracsagyctggcsgcsttyccrctgcarctggcsgtkccrttygcatgactcgaccatccgatttttt

SEQ ID NO: 52 - DNA, 85 bp, Artificial Sequence
  AC12-6
yggyggrtayggcagytgsgtrtartarctsgcsgcsgtsgcyggcagcatsgcrttrctgcatgactcgaccatccgatttttt

SEQ ID NO: 53 - DNA, 85 bp, Artificial Sequence
  AC12-7
ayacsaaycaraaytaygaygaytaygargcsgargcstayagyaaygcsatgctgccrggcatgactcgaccatccgatttttt

SEQ ID NO: 54 - DNA, 85 bp, Artificial Sequence
  AC12-8
rtcrtcrtarttytgrttsgtrtcyggrtayttcagrcayttytggtgatgatgatggtggcatgactcgaccatccgatttttt

SEQ ID NO: 55 - DNA, 85 bp, Artificial Sequence
  AC12-9
ctgtaacgggtcaatctggcccttttttcatatgcaccaccatcatcatcaccaraartggcatgactcgaccatccgatttttt

SEQ ID NO: 56 - DNA, 85 bp, Artificial Sequence
  AF4-1
gcsgaycgyctggtkcgyacsggyaaycayatygaytgygaaaccgtgcgtttccagtctgcatgactcgaccatccgatttttt

SEQ ID NO: 57 - DNA, 85 bp, Artificial Sequence
  AF4-2
gmaccagrcgrtcsgcytgrtcccacagytcrtgsgcrctrttcagrtarctcagraaytgcatgactcgaccatccgatttttt

SEQ ID NO: 58 - DNA, 85 bp, Artificial Sequence
  AF4-3
rgayatycayaayatgctgtgyaarcaraaygarttyctgagytayctgaayagygcscagcatgactcgaccatccgatttttt

SEQ ID NO: 59 - DNA, 85 bp, Artificial Sequence
  AF4-4
tgyttrcacagcatrttrtgratrtcyggyggmacratrcgrccyggyggsgtrttrctrgcatgactcgaccatccgatttttt

SEQ ID NO: 60 - DNA, 85 bp, Artificial Sequence
  AF4-5
agyagyatyagyccragyaayagygtkggyagycarggyagyggyagyaayacsccrccrgcatgactcgaccatccgatttttt

SEQ ID NO: 61 - DNA, 85 bp, Artificial Sequence
  AF4-6
trttrctyggrctratrctrctyggsgtrttrccrttsgcratrtcrccrcgrccmacrcgcatgactcgaccatccgatttttt

SEQ ID NO: 62 - DNA, 85 bp, Artificial Sequence
  AF4-7
cgyaargaytgycgygcsatyatyaayagyctgacsgayttyttycgygtkggycgyggygcatgactcgaccatccgatttttt

SEQ ID NO: 63 - DNA, 85 bp, Artificial Sequence
  AF4-8
tsgcrcgrcartcyttrcgrcgcagyttrtacagyttcagrctratcagrctytgrcarcgcatgactcgaccatccgatttttt

SEQ ID NO: 64 - DNA, 85 bp, Artificial Sequence
  AF4-9
gtcacatatgcaccaccatcatcatcacctgagyctgcgytgycaragyctgatyagyctgcatgactcgaccatccgatttttt

SEQ ID NO: 65 - DNA, 85 bp, Artificial Sequence
  AG3-1
carggytayagyacscarctggtkacsagyacsaartgygaaaccgtgcgtttccagtctgcatgactcgaccatccgatttttt

SEQ ID NO: 66 - DNA, 85 bp, Artificial Sequence
  AG3-2
cagytgsgtrctrtarccytgsgtrttrcasgtyggcagsgcraayggrttraarctcaggcatgactcgaccatccgatttttt

SEQ ID NO: 67 - DNA, 85 bp, Artificial Sequence
  AG3-3
yccrggyagygtkttyaayagyacsagycgygtkagyagyctgagyttyaayccrttygcgcatgactcgaccatccgatttttt

SEQ ID NO: 68 - DNA, 85 bp, Artificial Sequence
  AG3-4
macrctrccyggrctrctrctcagsgtrcccatrttyggsgcyggsgcrtarccrttsgcgcatgactcgaccatccgatttttt

SEQ ID NO: 69 - DNA, 85 bp, Artificial Sequence
  AG3-5
gytayggygcsacsgcsgcsagygcsgcsgtkgcsgcsacsgcsaayggytaygcsccrggcatgactcgaccatccgatttttt

SEQ ID NO: 70 - DNA, 85 bp, Artificial Sequence
  AG3-6
gcsgtsgcrccrtarctrccrccrctrctrtgyggsgtrctsgcrctrctrcartarccrgcatgactcgaccatccgatttttt

SEQ ID NO: 71 - DNA, 85 bp, Artificial Sequence
  AG3-7
aygaytaycarcgygcscaragyagyagygtkagyccrcgyggyggytaytgyagyagyggcatgactcgaccatccgatttttt

SEQ ID NO: 72 - DNA, 85 bp, Artificial Sequence
  AG3-8
sgcrcgytgrtartcrtcytcsgtccaytgrccgtgatgatgatggtggtgcatatgcgggcatgactcgaccatccgatttttt

SEQ ID NO: 73 - DNA, 85 bp, Artificial Sequence
  AR1-1
aayaarttyccrtgyatgttytgygaraaragyttytgygaaaccgtgcgtttccagtctgcatgactcgaccatccgatttttt

SEQ ID NO: 74 - DNA, 85 bp, Artificial Sequence
  AR1-2
rcaraacatrcayggraayttrttsgcsgtsgtyggsgtmacmacyttyggytcytcsgcgcatgactcgaccatccgatttttt

SEQ ID NO: 75 - DNA, 85 bp, Artificial Sequence
  AR1-3
gygaracscarctggaygcscarccrcarctgctgctggarccrgcsgargarccraarggcatgactcgaccatccgatttttt

SEQ ID NO: 76 - DNA, 85 bp, Artificial Sequence
  AR1-4
crtccagytgsgtytcrctrccrtcytcrctccarttmacrtcrcartcratsgccagragcatgactcgaccatccgatttttt

SEQ ID NO: 77 - DNA, 85 bp, Artificial Sequence
  AR1-5
ctggtkgarctgaarcaracsacsctgtgyagycarttyctggcsatygaytgygaygtkgcatgactcgaccatccgatttttt

SEQ ID NO: 78 - DNA, 85 bp, Artificial Sequence
  AR1-6
sgtytgyttcagytcmaccagyttccaraarttrtgraarctytccagytgrctrcarcagcatgactcgaccatccgatttttt

SEQ ID NO: 79 - DNA, 85 bp, Artificial Sequence
  AR1-7
yctgagycgygaygaygcsatyagyacstgyatytgyacsgartgytgyagycarctggagcatgactcgaccatccgatttttt

SEQ ID NO: 80 - DNA, 85 bp, Artificial Sequence
  AR1-8
tcrtcrcgrctcagrcacagrtacaggtgatgatgatggtggtgcatatgtcaagcgaaagcatgactcgaccatccgatttttt

SEQ ID NO: 81 - DNA, 85 bp, Artificial Sequence
  B11-1
gargaygtkgaraaraaragyccrgcsaarttyccrccrgaaaccgtgcgtttccagtctgcatgactcgaccatccgatttttt

SEQ ID NO: 82 - DNA, 85 bp, Artificial Sequence
  B11-2
ggrctyttyttytcmacrtcytcytcytcrtcytcrtcrtgrtgytcmacsgtytcyggcgcatgactcgaccatccgatttttt

SEQ ID NO: 83 - DNA, 85 bp, Artificial Sequence
  B11-3
yacsccrcayctgaaragyagyttyagyatyaayagyatyctgccrgaracsgtkgarcagcatgactcgaccatccgatttttt

SEQ ID NO: 84 - DNA, 85 bp, Artificial Sequence
  B11-4
rctrctyttcagrtgyggsgtrcgsgccatyttcatcagrttsgtcagrttcagrccmacgcatgactcgaccatccgatttttt

SEQ ID NO: 85 - DNA, 85 bp, Artificial Sequence
  B11-5
crccracsacscaycayagygcsctgcaragyccrcayccrgtkggyctgaayctgacsagcatgactcgaccatccgatttttt

SEQ ID NO: 86 - DNA, 85 bp, Artificial Sequence
  B11-6
tgrtgsgtsgtyggyggcagyggrtgrtgrtgrtgytgraartgcagrctrtgsgcrctrgcatgactcgaccatccgatttttt

SEQ ID NO: 87 - DNA, 85 bp, Artificial Sequence
  B11-7
catggtkaaratygargarggyctgccragyagygaratyagygcscayagyctgcayttgcatgactcgaccatccgatttttt

SEQ ID NO: 88 - DNA, 85 bp, Artificial Sequence
  B11-8
ccytcytcratyttmaccatgtgatgatgatggtggtgcatatggacaacactagcatgtgcatgactcgaccatccgatttttt

SEQ ID NO: 89 - DNA, 85 bp, Artificial Sequence
  D5-1
cayctggcsggyagytaygcsctggaygcsatggayagygaaaccgtgcgtttccagtctgcatgactcgaccatccgatttttt

SEQ ID NO: 90 - DNA, 85 bp, Artificial Sequence
  D5-2
crtarctrccsgccagrtgytgytgrtgrtgccacatytgrctytgsgtsgcytgytgcagcatgactcgaccatccgatttttt

SEQ ID NO: 91 - DNA, 85 bp, Artificial Sequence
  D5-3
csagytaygcsccrggyatggtkctggargaycargayccratgatgcarcargcsacscgcatgactcgaccatccgatttttt

SEQ ID NO: 92 - DNA, 85 bp, Artificial Sequence
  D5-4
ccyggsgcrtarctsgcrtgcagmacsgcsgcrtayttytcytcratytgrccsgcrtargcatgactcgaccatccgatttttt

SEQ ID NO: 93 - DNA, 85 bp, Artificial Sequence
  D5-5
aytgyatgtayccracsgcscargcscargcsccrgtkcayggytaygcsggycaratyggcatgactcgaccatccgatttttt

SEQ ID NO: 94 - DNA, 85 bp, Artificial Sequence
  D5-6
gcsgtyggrtacatrcartccagrttsgtrtacatcatrccrttytcgtgatgatgatgggcatgactcgaccatccgatttttt

SEQ ID NO: 95 - DNA, 85 bp, Artificial Sequence
  D5-7
ctgccggaatggtactggaccgcacaaaacatatgcaccaccatcatcatcacgaraayggcatgactcgaccatccgatttttt

SEQ ID NO: 96 - DNA, 85 bp, Artificial Sequence
  F9-1
gcscaycaygtkttyaayaarggycgytayaargtktgygaaaccgtgcgtttccagtctgcatgactcgaccatccgatttttt

SEQ ID NO: 97 - DNA, 85 bp, Artificial Sequence
  F9-2
rccyttrttraamacrtgrtgsgcrctrctcagyttcagcagytcytcyttratcagrcggcatgactcgaccatccgatttttt

SEQ ID NO: 98 - DNA, 85 bp, Artificial Sequence
  F9-3
yagygarctggcsgtkcaraargcsaarcgygaratyacscgyctgatyaargargarctgcatgactcgaccatccgatttttt

SEQ ID NO: 99 - DNA, 85 bp, Artificial Sequence
  F9-4
gmacsgccagytcrctrcarctytcratsgccagrtacagyttrcgytcrccrtcyggyggcatgactcgaccatccgatttttt

SEQ ID NO: 100 - DNA, 85 bp, Artificial Sequence
  F9-5
rgcsggyctgacsgtkcgyggyacstaygtkccrcarggyaaraayccrccrgayggygagcatgactcgaccatccgatttttt

SEQ ID NO: 101 - DNA, 85 bp, Artificial Sequence
  F9-6
cgmacsgtcagrccsgcytcrctrtaytcrctratytgsgccagsgcytcyttrctsgtmgcatgactcgaccatccgatttttt

SEQ ID NO: 102 - DNA, 85 bp, Artificial Sequence
  F9-7
cctggaratyaaygayttyccrcarcargcscgytggaargtkacsagyaargargcsctgcatgactcgaccatccgatttttt

SEQ ID NO: 103 - DNA, 85 bp, Artificial Sequence
  F9-8
yggraartcrttratytccaggtgatgatgatggtggtgcatatgcgattctctaaaaaggcatgactcgaccatccgatttttt

SEQ ID NO: 104 - DNA, 85 bp, Artificial Sequence
  K1-1
ctgggygarctgcayaaygcsgcsgtkgcsgcsgcstgygaaaccgtgcgtttccagtctgcatgactcgaccatccgatttttt

SEQ ID NO: 105 - DNA, 85 bp, Artificial Sequence
  K1-2
sgcrttrtgcagytcrcccagrctrctcagrccrcgrctrcccagraartcsgcrctrccgcatgactcgaccatccgatttttt

SEQ ID NO: 106 - DNA, 85 bp, Artificial Sequence
  K1-3
aaytgygcsagygcsttycayctggcsggyctgggyctgggyagygcsgayttyctgggygcatgactcgaccatccgatttttt

SEQ ID NO: 107 - DNA, 85 bp, Artificial Sequence
  K1-4
tgraasgcrctsgcrcarttmacrtayggrtcrtgraarctrctsgcrctrttsgtrcgygcatgactcgaccatccgatttttt

SEQ ID NO: 108 - DNA, 85 bp, Artificial Sequence
  K1-5
yccrggytayatggarcarctgtayagyctgcarcgyacsaayagygcsagyagyttycagcatgactcgaccatccgatttttt

SEQ ID NO: 109 - DNA, 85 bp, Artificial Sequence
  K1-6
gytgytccatrtarccyggratrcgrtasgcyggrtgraartcrtggtgatgatgatggtgcatgactcgaccatccgatttttt

SEQ ID NO: 110 - DNA, 85 bp, Artificial Sequence
  K1-7
ttaagccaggtggttcgtaattcgcaccatatgcaccaccatcatcatcaccaygayttygcatgactcgaccatccgatttttt

SEQ ID NO: 111 - DNA, 85 bp, Artificial Sequence
  K3-1
aayctggtkatygcscgyggyggyctggtkgaygtktgygaaaccgtgcgtttccagtctgcatgactcgaccatccgatttttt

SEQ ID NO: 112 - DNA, 85 bp, Artificial Sequence
  K3-2
crcgsgcratmaccagrttrtacagytcrtacagrtccagmacrctyttsgccatratyggcatgactcgaccatccgatttttt

SEQ ID NO: 113 - DNA, 85 bp, Artificial Sequence
  K3-3
tyagyttyatgcaraarcgyggyacsccratyaaycgyctgccratyatggcsaaragyggcatgactcgaccatccgatttttt

SEQ ID NO: 114 - DNA, 85 bp, Artificial Sequence
  k3-4
rcgyttytgcatraarctraacagrtcrtccagraaytcyttrcgyttyggrtcrtcrttgcatgactcgaccatccgatttttt

SEQ ID NO: 115 - DNA, 85 bp, Artificial Sequence
  k3-5
tygargarcarttyaarcargtkcgycarctgtaygaratyaaygaygayccraarcgyagcatgactcgaccatccgatttttt

SEQ ID NO: 116 - DNA, 85 bp, Artificial Sequence
  k3-6
grcgmacytgyttraaytgytcytcraarctccarccrttrttytgytgrctrctrttrcgcatgactcgaccatccgatttttt

SEQ ID NO: 117 - DNA, 85 bp, Artificial Sequence
  k3-7
tcatcatcacgcsagyaayagyagyacsagyagygargcsagyaayagyagycarcaraagcatgactcgaccatccgatttttt

SEQ ID NO: 118 - DNA, 85 bp, Artificial Sequence
  k3-8
trttrctsgcgtgatgatgatggtggtgcatatgctacaaaagtcccgcccaacaaccccgcatgactcgaccatccgatttttt

SEQ ID NO: 119 - DNA, 85 bp, Artificial Sequence
  k4-1
tayctgaayccrcayaayatggcsgcsgtkgcsgcstgygaaaccgtgcgtttccagtctgcatgactcgaccatccgatttttt

SEQ ID NO: 120 - DNA, 85 bp, Artificial Sequence
  k4-2
ccatrttrtgyggrttcagrtaraarttsgcyggraarccyggyggcagcagrccrctrcgcatgactcgaccatccgatttttt

SEQ ID NO: 121 - DNA, 85 bp, Artificial Sequence
  k4-3
gaygayaarttygtkgaycarccrccrccrgcsaarcgygtkggyagyggyctgctgccrgcatgactcgaccatccgatttttt

SEQ ID NO: 122 - DNA, 85 bp, Artificial Sequence
  k4-4
gytgrtcmacraayttrtcrtcytgrtcmacyggytcyggsgtyggytcrcgytcrcgytgcatgactcgaccatccgatttttt

SEQ ID NO: 123 - DNA, 85 bp, Artificial Sequence
  k4-5
gaayctgagygayagyccrccraayctgacsaayatyaarcgygarcgygarcgygarccgcatgactcgaccatccgatttttt

SEQ ID NO: 124 - DNA, 85 bp, Artificial Sequence
  k4-6
ggrctrtcrctcagrttcagsgcytcrcgytgytgyggrctsgtrtgsgcrctgtgatgagcatgactcgaccatccgatttttt

SEQ ID NO: 125 - DNA, 85 bp, Artificial Sequence
  k4-7
gaccgggaagaatcactgcatggtcatatgcaccaccatcatcatcacagygcscayacsgcatgactcgaccatccgatttttt

SEQ ID NO: 126 - DNA, 85 bp, Artificial Sequence
  k5-1
tgyggyggycayaartgyatyaarccrtgycaygartgygaaaccgtgcgtttccagtctgcatgactcgaccatccgatttttt

SEQ ID NO: 127 - DNA, 85 bp, Artificial Sequence
  k5-2
yttrtgrccrccrcayggcagyggyttrccrcasgccatrccrcarctraarttyggytggcatgactcgaccatccgatttttt

SEQ ID NO: 128 - DNA, 85 bp, Artificial Sequence
  k5-3
ycayggyaaycaygarctgcgyaaracsatyccrtgyagycarccraayttyagytgygggcatgactcgaccatccgatttttt

SEQ ID NO: 129 - DNA, 85 bp, Artificial Sequence
  k5-4
gytcrtgrttrccrtgrcacagyttsgtsgtraaratcatrcayggyggrcasgtyggrcgcatgactcgaccatccgatttttt

SEQ ID NO: 130 - DNA, 85 bp, Artificial Sequence
  k5-5
gyatycayccrtgygaycayccrccrcarcayaaytgycayagyggyccracstgyccrcgcatgactcgaccatccgatttttt

SEQ ID NO: 131 - DNA, 85 bp, Artificial Sequence
  k5-6
tgrtcrcayggrtgratrcgrctrcayggcagyttrcaratyggyttyttsgtrccrcaygcatgactcgaccatccgatttttt

SEQ ID NO: 132 - DNA, 85 bp, Artificial Sequence
  k5-7
gtaytgygartgyggygcsgargtkatytayccrccrgtkccrtgyggyacsaaraarccgcatgactcgaccatccgatttttt

SEQ ID NO: 133 - DNA, 85 bp, Artificial Sequence
  k5-8
sgcrccrcaytcrcartacagytcytcraarctgtgatgatgatggtggtgcatatgtacgcatgactcgaccatccgatttttt

SEQ ID NO: 134 - DNA, 85 bp, Artificial Sequence
  l5-1
acsgcsatgaayctgcaragyatyaayttyaayagytgygaaaccgtgcgtttccagtctgcatgactcgaccatccgatttttt

SEQ ID NO: 135 - DNA, 85 bp, Artificial Sequence
  l5-2
ttratrctytgcagrttcatsgcsgtsgtyggrttmacrttratsgtrccsgtrccsgcrgcatgactcgaccatccgatttttt

SEQ ID NO: 136 - DNA, 85 bp, Artificial Sequence
  l5-3
carcgytayatygargcsacsggyggyggygcsggygcsggyacsggygcsggyacsggygcatgactcgaccatccgatttttt

SEQ ID NO: 137 - DNA, 85 bp, Artificial Sequence
  l5-4
tsgcytcratrtarcgytgrcgsgtcatrctrtgmacrttrccyggrctrtccatrtarcgcatgactcgaccatccgatttttt

SEQ ID NO: 138 - DNA, 85 bp, Artificial Sequence
  l5-5
yagycayccrgcsatyggygtkatgagyctgttygayccrcgytayatggayagyccrgggcatgactcgaccatccgatttttt

SEQ ID NO: 139 - DNA, 85 bp, Artificial Sequence
  l5-6
ratsgcyggrtgrctrttrccrccgtgatgatgatggtggtgcatatgcgccgattcgctgcatgactcgaccatccgatttttt

SEQ ID NO: 140 - DNA, 85 bp, Artificial Sequence
  m6-1
ggyaayggyaaygcsggyggygtkcaragyggygtktgygaaaccgtgcgtttccagtctgcatgactcgaccatccgatttttt

SEQ ID NO: 141 - DNA, 85 bp, Artificial Sequence
  m6-2
gcrttrccrttrccrccrctmacrctmacrttrccrccsgcrctrccrccrcgrtgrctmgcatgactcgaccatccgatttttt

SEQ ID NO: 142 - DNA, 85 bp, Artificial Sequence
  m6-3
ayagycgygtkggyggytayctggayacsagyggyggyagyccrgtkagycaycgyggyggcatgactcgaccatccgatttttt

SEQ ID NO: 143 - DNA, 85 bp, Artificial Sequence
  m6-4
ccrccmacrcgrctrtcyggsgtrcasgcrctyggrcgmacyggcatrccrccsgcyggrgcatgactcgaccatccgatttttt

SEQ ID NO: 144 - DNA, 85 bp, Artificial Sequence
  m6-5
csggyaaygcsaayggyggyaaygcsgcsaaygcsaayggycaraayaayccrgcsggyggcatgactcgaccatccgatttttt

SEQ ID NO: 145 - DNA, 85 bp, Artificial Sequence
  m6-6
ccrttsgcrttrccsgcrccrccgtgatgatgatggtggtgcatatgcctccggcgtctggcatgactcgaccatccgatttttt

SEQ ID NO: 146 - DNA, 85 bp, Artificial Sequence
  bcd_d2-1
aaygcsgcsggyaayagycarttygcstaytgyttyaaygaaaccgtgcgtttccagtctgcatgactcgaccatccgatttttt

SEQ ID NO: 147 - DNA, 85 bp, Artificial Sequence
  bcd_d2-2
rccsgcsgcrttrcgrtgyggrttrtasgcytgcatratsgtrtccatsgtrcaytgrtagcatgactcgaccatccgatttttt

SEQ ID NO: 148 - DNA, 85 bp, Artificial Sequence
  bcd_d2-3
tgggyatgggyggygtkgcsatgggygaragyaaycartaycartgyacsatggayacsagcatgactcgaccatccgatttttt

SEQ ID NO: 149 - DNA, 85 bp, Artificial Sequence
  bcd_d2-4
rccrcccatrcccagyggyggytgyggrccytgyggyggrctyggyttrccraayttsgcgcatgactcgaccatccgatttttt

SEQ ID NO: 150 - DNA, 85 bp, Artificial Sequence
  bcd_d2-5
yatycgygcsctggcsggyacsggyaaycgyggygcsgcsttygcsaarttyggyaarccgcatgactcgaccatccgatttttt

SEQ ID NO: 151 - DNA, 85 bp, Artificial Sequence
  bcd_d2-6
csgtrccsgccagsgcrcgratrccsgtrctcatrtcrtcrctrctrccrtcrtcrcarcgcatgactcgaccatccgatttttt

SEQ ID NO: 152 - DNA, 85 bp, Artificial Sequence
  bcd_d2-7
accatcatcatcacatyctggarccrctgaarggyctggayaaragytgygaygayggyagcatgactcgaccatccgatttttt

SEQ ID NO: 153 - DNA, 85 bp, Artificial Sequence
  bcd_d2-8
tccagratgtgatgatgatggtggtgcatatgggcccgccgcgtggaacacacattcctagcatgactcgaccatccgatttttt

SEQ ID NO: 154 - DNA, 85 bp, Artificial Sequence
  cad_d1-1
caraayaaraarggyagygayccraaygtkatgggygtkgaaaccgtgcgtttccagtctgcatgactcgaccatccgatttttt

SEQ ID NO: 155 - DNA, 85 bp, Artificial Sequence
  cad_d1-2
ggrtcrctrccyttyttrttytgyttrcgytcyttsgcrcgrcgrttytgraaccaratygcatgactcgaccatccgatttttt

SEQ ID NO: 156 - DNA, 85 bp, Artificial Sequence
  cad_d1-3
garctggcscaracsctgagyctgagygarcgycargtkaaratytggttycaraaycgygcatgactcgaccatccgatttttt

SEQ ID NO: 157 - DNA, 85 bp, Artificial Sequence
  cad_d1-4
gsgtytgsgccagytcrctyttrcgrcgratsgtratrtarcgrctsgtrcartaytcytgcatgactcgaccatccgatttttt

SEQ ID NO: 158 - DNA, 85 bp, Artificial Sequence
  cad_d1-5
ycgygtkgtktayacsgayttycarcgyctggarctggaraargartaytgyacsagycggcatgactcgaccatccgatttttt

SEQ ID NO: 159 - DNA, 85 bp, Artificial Sequence
  cad_d1-6
csgtrtamacmacrcgrtayttrtcyttsgtrcgsgtyttrccyggytgyggytgsgcyggcatgactcgaccatccgatttttt

SEQ ID NO: 160 - DNA, 85 bp, Artificial Sequence
  cad_d1-7
yaarccrccrtayttygaytggatgaaraarccrgcstayccrgcscarccrcarccrgggcatgactcgaccatccgatttttt

SEQ ID NO: 161 - DNA, 85 bp, Artificial Sequence
  cad_d1-8
rtcraartayggyggyttrctyggrctsgtrcggtgatgatgatggtggtgcatatggaggcatgactcgaccatccgatttttt

SEQ ID NO: 162 - DNA, 85 bp, Artificial Sequence
  hb_d2-1
agyggyagycgyaaragyaaygtkgcsgcsgtkgcsccrgaaaccgtgcgtttccagtctgcatgactcgaccatccgatttttt

SEQ ID NO: 163 - DNA, 85 bp, Artificial Sequence
  hb_d2-2
trcgrctrccrctrccrctrccrccrctsgcratyggrccrccrttyttrctyttyggrcgcatgactcgaccatccgatttttt

SEQ ID NO: 164 - DNA, 85 bp, Artificial Sequence
  hb_d2-3
ayccragyctggtkatygaygtktayggyacscgycgyggyccraaragyaaraayggyggcatgactcgaccatccgatttttt

SEQ ID NO: 165 - DNA, 85 bp, Artificial Sequence
  hb_d2-4
tcratmaccagrctyggrttyggsgtrccrtcytcrtccagmaccatrccyggyttrtgrgcatgactcgaccatccgatttttt

SEQ ID NO: 166 - DNA, 85 bp, Artificial Sequence
  hb_d2-5
acsaartaytgycayagyttyaarctgcayctgcgyaartayggycayaarccrggyatggcatgactcgaccatccgatttttt

SEQ ID NO: 167 - DNA, 85 bp, Artificial Sequence
  hb_d2-6
traarctrtgrcartayttsgtsgcrtartcrcartcsgcrcarcgrtaytgrtamacrcgcatgactcgaccatccgatttttt

SEQ ID NO: 168 - DNA, 85 bp, Artificial Sequence
  hb_d2-7
aaragyatgctgaayagycaycgyaaragycayagyagygtktaycartaycgytgygcsgcatgactcgaccatccgatttttt

SEQ ID NO: 169 - DNA, 85 bp, Artificial Sequence
  hb_d2-8
grtgrctrttcagcatrctyttrttmacrcasgtrtarctrcayttrtcrcaytgraayggcatgactcgaccatccgatttttt

SEQ ID NO: 170 - DNA, 85 bp, Artificial Sequence
  hb_d2-9
tatgcaccaccatcatcatcacaarcayaaraaycaraarccrttycartgygayaartggcatgactcgaccatccgatttttt

SEQ ID NO: 171 - DNA, 85 bp, Artificial Sequence
  hb_d2-10
tgatgatgatggtggtgcatatgcgtcctgcgaattcattcgatcgatgatggcgtcctggcatgactcgaccatccgatttttt

SEQ ID NO: 172 - DNA, 85 bp, Artificial Sequence
  lab_d1-1
ctgacscarcayagyacsagygtkatyagygaraarccrgaaaccgtgcgtttccagtctgcatgactcgaccatccgatttttt

SEQ ID NO: 173 - DNA, 85 bp, Artificial Sequence
  lab_d1-2
sgtrctrtgytgsgtcagratrtcsgcyggratcagrccytcyttmacrcgyttyttytggcatgactcgaccatccgatttttt

SEQ ID NO: 174 - DNA, 85 bp, Artificial Sequence
  lab_d1-3
aracscargtkaaratytggttycaraaycgycgyatgaarcaraaraarcgygtkaarggcatgactcgaccatccgatttttt

SEQ ID NO: 175 - DNA, 85 bp, Artificial Sequence
  lab_d1-4
accaratyttmacytgsgtytcrttcagytgcagsgtrttsgcratytcratrcgrcgsggcatgactcgaccatccgatttttt

SEQ ID NO: 176 - DNA, 85 bp, Artificial Sequence
  lab_d1-5
gacsgarctggaraargarttycayttyaaycgytayctgacscgygcscgycgyatygagcatgactcgaccatccgatttttt

SEQ ID NO: 177 - DNA, 85 bp, Artificial Sequence
  lab_d1-6
raaytcyttytccagytcsgtcagytgyttrttsgtraarttsgtrcgrccrctrttrttgcatgactcgaccatccgatttttt

SEQ ID NO: 178 - DNA, 85 bp, Artificial Sequence
  lab_d1-7
gyagyggyagyggyctgagyagytgyagyctgagyagyaayacsaayaayagyggycgyagcatgactcgaccatccgatttttt

SEQ ID NO: 179 - DNA, 85 bp, Artificial Sequence
  lab_d1-8
grccrctrccrctrcccagrccmacrcccatrccrttygggtgatgatgatggtggtgcagcatgactcgaccatccgatttttt

SEQ ID NO: 180 - DNA, 85 bp, Artificial Sequence
  lab_d1-9
acctctctacatccctcgcacggcagtcgttaggtaacatatgcaccaccatcatcatcagcatgactcgaccatccgatttttt

SEQ ID NO: 181 - DNA, 85 bp, Artificial Sequence
  lab_d2-1
caygaytaycaratgaayggycarctggayatgtgycgygaaaccgtgcgtttccagtctgcatgactcgaccatccgatttttt

SEQ ID NO: 182 - DNA, 85 bp, Artificial Sequence
  lab_d2-2
tgrccrttcatytgrtartcrtgcatrctsgcratrccrctsgcyggcagyttyggsgcygcatgactcgaccatccgatttttt

SEQ ID NO: 183 - DNA, 85 bp, Artificial Sequence
  lab_d2-3
tayaartggatgcarctgaarcgyaaygtkccraarccrcargcsccraarctgccrgcsgcatgactcgaccatccgatttttt

SEQ ID NO: 184 - DNA, 85 bp, Artificial Sequence
  lab_d2-4
rcgyttcagytgcatccayttrtasgtyggratrctrctrctrtgrttyggrctyttrctgcatgactcgaccatccgatttttt

SEQ ID NO: 185 - DNA, 85 bp, Artificial Sequence
  lab_d2-5
acsagygcsagyaayggygcscayccrgcsagyacscaragyaaragyccraaycayagygcatgactcgaccatccgatttttt

SEQ ID NO: 186 - DNA, 85 bp, Artificial Sequence
  lab_d2-6
ccrttrctsgcrctsgtrctrctrctsgtrctrctrccyggrctratmacrccrccrcgygcatgactcgaccatccgatttttt

SEQ ID NO: 187 - DNA, 85 bp, Artificial Sequence
  lab_d2-7
caycaycaraayagygtkagyccraayggyggyatgaaycgycarcarcgyggyggygtkgcatgactcgaccatccgatttttt

SEQ ID NO: 188 - DNA, 85 bp, Artificial Sequence
  lab_d2-8
ctmacrctrttytgrtgrtgytgytgrtgsgcsgcgtgatgatgatggtggtgcatatgagcatgactcgaccatccgatttttt

SEQ ID NO: 189 - DNA, 85 bp, Artificial Sequence
  pb_d2-1
ccragyctggtkagyttycgycgygayagygaygcsagygaaaccgtgcgtttccagtctgcatgactcgaccatccgatttttt

SEQ ID NO: 190 - DNA, 85 bp, Artificial Sequence
  pb_d2-2
cgraarctmaccagrctyggsgtrctrttytcrtarccraayggrctsgcyttrctrctsgcatgactcgaccatccgatttttt

SEQ ID NO: 191 - DNA, 85 bp, Artificial Sequence
  pb_d2-3
araargaygayggycargtkatyaaraargargcsgtkagyacsagyagyaargcsagycgcatgactcgaccatccgatttttt

SEQ ID NO: 192 - DNA, 85 bp, Artificial Sequence
  pb_d2-4
ratmacytgrccrtcrtcyttyttyttmacyttratyggrctrctytcytcratrtcytcgcatgactcgaccatccgatttttt

SEQ ID NO: 193 - DNA, 85 bp, Artificial Sequence
  pb_d2-5
gygcsgayagyagygtkgcsagyagygtkagyctggaygargayatygargaragyagycgcatgactcgaccatccgatttttt

SEQ ID NO: 194 - DNA, 85 bp, Artificial Sequence
  pb_d2-6
acrctrctrtcsgcrctratmacrttrctsgcrctmacsgtsgtrctrcccatcagrttrgcatgactcgaccatccgatttttt

SEQ ID NO: 195 - DNA, 85 bp, Artificial Sequence
  pb_d2-7
yaayctgacsccraayagyagyctggaracsggyatyagyagyaayctgatgggyagyacgcatgactcgaccatccgatttttt

SEQ ID NO: 196 - DNA, 85 bp, Artificial Sequence
  pb_d2-8
rctrctrttyggsgtcagrttrccsgcrctyggrttrttrttsgtsgcrctyggsgtrttgcatgactcgaccatccgatttttt

SEQ ID NO: 197 - DNA, 85 bp, Artificial Sequence
  pb_d2-9
gaygayatyccrgayagyacsagyaayagycgyggycayaayaayaayacsccragygcsgcatgactcgaccatccgatttttt

SEQ ID NO: 198 - DNA, 85 bp, Artificial Sequence
  pb_d2-10
ctsgtrctrtcyggratrtcrtcrctyggcagytcrcarccytgrcarctyttyttrctrgcatgactcgaccatccgatttttt

SEQ ID NO: 199 - DNA, 85 bp, Artificial Sequence
  pb_d2-11
acgaatcatatgcaccaccatcatcatcacaayagyaayagyaaraaragytgycarggygcatgactcgaccatccgatttttt

SEQ ID NO: 200 - DNA, 85 bp, Artificial Sequence
  dfd_d1-1
gcsagygcsggyggytayagyagyaaytaygcsaaygcsgaaaccgtgcgtttccagtctgcatgactcgaccatccgatttttt

SEQ ID NO: 201 - DNA, 85 bp, Artificial Sequence
  dfd_d1-2
crccsgcrctsgcytgrtgrtgrccrctratsgcrctrttrctrtgrtgrtgyggcagrcgcatgactcgaccatccgatttttt

SEQ ID NO: 202 - DNA, 85 bp, Artificial Sequence
  dfd_d1-3
caycayaayccrcayagycayagycayagycayacscayagyctgccrcaycaycayagygcatgactcgaccatccgatttttt

SEQ ID NO: 203 - DNA, 85 bp, Artificial Sequence
  dfd_d1-4
rctrtgyggrttrtgrtgrtgsgccatrtartcrctmaccatrtcsgcyggrtgcatrctgcatgactcgaccatccgatttttt

SEQ ID NO: 204 - DNA, 85 bp, Artificial Sequence
  dfd_d1-5
yagygtkggyggyggyggygcsggyggyatgacsggycayccrcayagyatgcayccrgcgcatgactcgaccatccgatttttt

SEQ ID NO: 205 - DNA, 85 bp, Artificial Sequence
  dfd_d1-6
ccrccrccrccmacrctrccmacrccsgcrccrccrccmacsgcrccrctcatrtgrccsgcatgactcgaccatccgatttttt

SEQ ID NO: 206 - DNA, 85 bp, Artificial Sequence
  dfd_d1-7
aycaycaytayaayggycaytayagyatgacsgcsagyacsggycayatgagyggygcsggcatgactcgaccatccgatttttt

SEQ ID NO: 207 - DNA, 85 bp, Artificial Sequence
  dfd_d1-8
trctrtartgrccrttrtartgrtgrtartcrtcsgccagyggyggraayttyggrtccagcatgactcgaccatccgatttttt

SEQ ID NO: 208 - DNA, 85 bp, Artificial Sequence
  dfd_d1-9
gttggaaacatatgcaccaccatcatcatcacggyaayggyctggayccraarttyccrcgcatgactcgaccatccgatttttt

SEQ ID NO: 209 - DNA, 85 bp, Artificial Sequence
  scr_d2-1
gcsaayggygaracsaarcgycarcgyacsagytayacsgaaaccgtgcgtttccagtctgcatgactcgaccatccgatttttt

SEQ ID NO: 210 - DNA, 85 bp, Artificial Sequence
  scr_d2-2
tsgtytcrccrttsgcrttmacsgtrctsgtrcccagrtgmacrcgyttcatccayggrtgcatgactcgaccatccgatttttt

SEQ ID NO: 211 - DNA, 85 bp, Artificial Sequence
  scr_d2-3
ycaraayagyggyaayggyaaraaraayccrccrcaratytayccrtggatgaarcgygtgcatgactcgaccatccgatttttt

SEQ ID NO: 212 - DNA, 85 bp, Artificial Sequence
  scr_d2-4
crttrccrctrttytgrctrctrccsgcytcrttrccrctrtcrctytcrctrtcrctrtgcatgactcgaccatccgatttttt

SEQ ID NO: 213 - DNA, 85 bp, Artificial Sequence
  scr_d2-5
crggyaaygtkaaygtkccratgcayagyccrggyggyggygayagygayagygaragyggcatgactcgaccatccgatttttt

SEQ ID NO: 214 - DNA, 85 bp, Artificial Sequence
  scr_d2-6
yggmacrttmacrttrccyggrccrccrctmacrccrctrccrctrtgrttgtgatgatggcatgactcgaccatccgatttttt

SEQ ID NO: 215 - DNA, 85 bp, Artificial Sequence
  scr_d2-7
aacttgcaggaatccaactgtactaacatatgcaccaccatcatcatcacaaycayagyggcatgactcgaccatccgatttttt

SEQ ID NO: 216 - DNA, 85 bp, Artificial Sequence
  antp_d1-1
ccrcargcscaracsaayggycarctgggygtkccrcargaaaccgtgcgtttccagtctgcatgactcgaccatccgatttttt

SEQ ID NO: 217 - DNA, 85 bp, Artificial Sequence
  antp_d1-2
sgtytgsgcytgyggcatmacrccrccmacytgrctrctyggrctrtcyggrcgrctrtagcatgactcgaccatccgatttttt

SEQ ID NO: 218 - DNA, 85 bp, Artificial Sequence
  antp_d1-3
aytayaayggycarggyatggaycarcarcarcarcaycargtktayagycgyccrgayagcatgactcgaccatccgatttttt

SEQ ID NO: 219 - DNA, 85 bp, Artificial Sequence
  antp_d1-4
trccytgrccrttrtartayggcatrcgrtcrtayggyggraarcgyggrtayggcatrtgcatgactcgaccatccgatttttt

SEQ ID NO: 220 - DNA, 85 bp, Artificial Sequence
  antp_d1-5
scarcaratgcaycaytayagycaraaygcsaaycaycarggyaayatgccrtayccrcggcatgactcgaccatccgatttttt

SEQ ID NO: 221 - DNA, 85 bp, Artificial Sequence
  antp_d1-6
trtartgrtgcatytgytgsgcrtccagrtcsgtmacrccrttrccyggrtartgrccrtgcatgactcgaccatccgatttttt

SEQ ID NO: 222 - DNA, 85 bp, Artificial Sequence
  antp_d1-7
cacsagytayttyacsaayagytayatgggygcsgayatgcaycayggycaytayccrgggcatgactcgaccatccgatttttt

SEQ ID NO: 223 - DNA, 85 bp, Artificial Sequence
  antp_d1-8
rctrttsgtraartarctsgtgtgatgatgatggtggtgcatatgcagctggaccccactgcatgactcgaccatccgatttttt

SEQ ID NO: 224 - DNA, 85 bp, Artificial Sequence
  lid_d2-1
atygaycgyctgaaygcsgcsgcsgtkgargcsgaraargaaaccgtgcgtttccagtctgcatgactcgaccatccgatttttt

SEQ ID NO: 225 - DNA, 85 bp, Artificial Sequence
  lid_d2-2
gcsgcrttcagrcgrtcratcagcagrctrctyggraayttyttsgtytcsgcytcyttrgcatgactcgaccatccgatttttt

SEQ ID NO: 226 - DNA, 85 bp, Artificial Sequence
  lid_d2-3
ayacsccracsagygtkacsctgcargarctgcargarctgtgyaargargcsgaracsagcatgactcgaccatccgatttttt

SEQ ID NO: 227 - DNA, 85 bp, Artificial Sequence
  lid_d2-4
sgtmacrctsgtyggsgtrtgsgcrtcmacratrtcrcgrcarcgrctcagccarcgytcgcatgactcgaccatccgatttttt

SEQ ID NO: 228 - DNA, 85 bp, Artificial Sequence
  lid_d2-5
ccrctgatgctgcaraarctgaargtkaargcscayagyttygarcgytggctgagycgygcatgactcgaccatccgatttttt

SEQ ID NO: 229 - DNA, 85 bp, Artificial Sequence
  lid_d2-6
ttytgcagcatcagyggcatytcrtccagsgtrtarcgrtaratcagsgtrtgyttytcygcatgactcgaccatccgatttttt

SEQ ID NO: 230 - DNA, 85 bp, Artificial Sequence
  lid_d2-7
ycaytayacsgtkctgtgyggytgygcsccrgaraarcayacsctgatytaycgytayacgcatgactcgaccatccgatttttt

SEQ ID NO: 231 - DNA, 85 bp, Artificial Sequence
  lid_d2-8
rccrcacagmacsgtrtartgrcgcagrcamacratcagyttrtcrttrcaytcrcagtggcatgactcgaccatccgatttttt

SEQ ID NO: 232 - DNA, 85 bp, Artificial Sequence
  lid_d2-9
ttactcaccggttaccagcgtcatatgcaccaccatcatcatcactgygartgyaaygaygcatgactcgaccatccgatttttt

SEQ ID NO: 233 - DNA, 85 bp, Artificial Sequence
  lilli_d1-1
ccracsgcsgcsagygcsacsacsagyctgccrggycargaaaccgtgcgtttccagtctgcatgactcgaccatccgatttttt

SEQ ID NO: 234 - DNA, 85 bp, Artificial Sequence
  lilli_d1-2
ctsgcsgcsgtyggrccsgcsgcrctrctsgcrctrctyggrctrtgrcccagrctyttsgcatgactcgaccatccgatttttt

SEQ ID NO: 235 - DNA, 85 bp, Artificial Sequence
  lilli_d1-3
ygcsggyaayccrcgyctgcarccraayctggcsccrcargcsaaragyctgggycayaggcatgactcgaccatccgatttttt

SEQ ID NO: 236 - DNA, 85 bp, Artificial Sequence
  lilli_d1-4
yggrttrccsgcrccyggsgcrctrcgrctratrccmacsgtytgrttrttratrtaytcgcatgactcgaccatccgatttttt

SEQ ID NO: 237 - DNA, 85 bp, Artificial Sequence
  lilli_d1-5
yacsgcsgcsctgggygarttyttygargcscgygartayatyaayaaycaracsgtkgggcatgactcgaccatccgatttttt

SEQ ID NO: 238 - DNA, 85 bp, Artificial Sequence
  lilli_d1-6
cagsgcsgcsgtratytcsgcrtcrccytcsgtcagrcgrcgyggytcrccraacagrctgcatgactcgaccatccgatttttt

SEQ ID NO: 239 - DNA, 85 bp, Artificial Sequence
  lilli_d1-7
atygarcgycarcarggyatycaratygaygaycgygaracsagyctgttyggygarccrgcatgactcgaccatccgatttttt

SEQ ID NO: 240 - DNA, 85 bp, Artificial Sequence
  lilli_d1-8
ccytgytgrcgytcratyttytcrcgytcrcgrcgyttrcgrcgytcrtaytcytccatrgcatgactcgaccatccgatttttt

SEQ ID NO: 241 - DNA, 85 bp, Artificial Sequence
  lilli_d1-9
tgcaccaccatcatcatcacctgtayagycaraaytayaayatggargartaygarcgycgcatgactcgaccatccgatttttt

SEQ ID NO: 242 - DNA, 85 bp, Artificial Sequence
  lilli_d1-10
tgatgatgatggtggtgcatatgagaagatcctggccgagtagctttcatccttgacaaagcatgactcgaccatccgatttttt

SEQ ID NO: 243 - DNA, 85 bp, Artificial Sequence
  lilli_d2-1
ggyctgaaracsctgcgygaygcsgtkagycayccracsgaaaccgtgcgtttccagtctgcatgactcgaccatccgatttttt

SEQ ID NO: 244 - DNA, 85 bp, Artificial Sequence
  lilli_d2-2
crcgcagsgtyttcagrccsgcytgmacrtarcgraamacytcrtgcatsgtrctrtgcagcatgactcgaccatccgatttttt

SEQ ID NO: 245 - DNA, 85 bp, Artificial Sequence
  lilli_d2-3
ayttyatycgygarctggaycaygaraayggyccrctgacsctgcayagyacsatgcayggcatgactcgaccatccgatttttt

SEQ ID NO: 246 - DNA, 85 bp, Artificial Sequence
  lilli_d2-4
tgrtccagytcrcgratraartcratrtgrttrccsgtrcgmaccagrcgrtcsgcytgrgcatgactcgaccatccgatttttt

SEQ ID NO: 247 - DNA, 85 bp, Artificial Sequence
  lilli_d2-5
ygarttyctgagytayctgaayagygcscaygarctgtgggaycargcsgaycgyctggtgcatgactcgaccatccgatttttt

SEQ ID NO: 248 - DNA, 85 bp, Artificial Sequence
  lilli_d2-6
gcrctrttcagrtarctcagraaytcrttytgyttrcacagcatrttrtgratrtcyggygcatgactcgaccatccgatttttt

SEQ ID NO: 249 - DNA, 85 bp, Artificial Sequence
  lilli_d2-7
gyggyagyaayacsccrccrggycgyatygtkccrccrgayatycayaayatgctgtgyagcatgactcgaccatccgatttttt

SEQ ID NO: 250 - DNA, 85 bp, Artificial Sequence
  lilli_d2-8
yggsgtrttrctrccrctrccytgrctrccmacrctrttrctyggrctratrctrctygggcatgactcgaccatccgatttttt

SEQ ID NO: 251 - DNA, 85 bp, Artificial Sequence
  lilli_d2-9
ccatcatcatcaccgyggygayatygcsaayggyaayacsccragyagyatyagyccraggcatgactcgaccatccgatttttt

SEQ ID NO: 252 - DNA, 85 bp, Artificial Sequence
  lilli_d2-10
rccrcggtgatgatgatggtggtgcatatggtactgtccttcttcccaccactagtattagcatgactcgaccatccgatttttt

SEQ ID NO: 253 - DNA, 85 bp, Artificial Sequence
  e75_d1-1
gayacsaayagyctgatggaygargcstayaarccrcaygaaaccgtgcgtttccagtctgcatgactcgaccatccgatttttt

SEQ ID NO: 254 - DNA, 85 bp, Artificial Sequence
  e75_d1-2
crtccatcagrctrttsgtrtcrtacagyggyggsgcytgcagmacrcgyttcagmacyggcatgactcgaccatccgatttttt

SEQ ID NO: 255 - DNA, 85 bp, Artificial Sequence
  e75_d1-3
cragyacsagyagycayctgaarcgycaratygtkgargayatgccrgtkctgaarcgyggcatgactcgaccatccgatttttt

SEQ ID NO: 256 - DNA, 85 bp, Artificial Sequence
  e75_d1-4
tgrctrctsgtrctyggytgyggrctrcgmacyggrctmacrctmacmacrctcagytgygcatgactcgaccatccgatttttt

SEQ ID NO: 257 - DNA, 85 bp, Artificial Sequence
  e75_d1-5
agygaygcsgcsgcsaaycayaaycargtkgtkcarcayccrcarctgagygtkgtkagygcatgactcgaccatccgatttttt

SEQ ID NO: 258 - DNA, 85 bp, Artificial Sequence
  e75_d1-6
csgcsgcrtcrctrcartccagsgcrtcrtcmacrctrctrcgyggrctrctrcarctrcgcatgactcgaccatccgatttttt

SEQ ID NO: 259 - DNA, 85 bp, Artificial Sequence
  e75_d1-7
aaygaraaraaygartgyaargcsgtkagyagyggyggyagyagyagytgyagyagyccrgcatgactcgaccatccgatttttt

SEQ ID NO: 260 - DNA, 85 bp, Artificial Sequence
  e75_d1-8
cyttrcaytcrttyttytcrttrccrctytcratrccrctrtcsgtyggrctrtccagytgcatgactcgaccatccgatttttt

SEQ ID NO: 261 - DNA, 85 bp, Artificial Sequence
  e75_d1-9
atcatcatcaccayctgacsgcsggygcsgcscgytaycgyaarctggayagyccracsggcatgactcgaccatccgatttttt

SEQ ID NO: 262 - DNA, 85 bp, Artificial Sequence
  e75_d1-10
sgcsgtcagrtggtgatgatgatggtggtgcatatgggctattcacattacgccgcgaatgcatgactcgaccatccgatttttt

SEQ ID NO: 263 - DNA, 85 bp, Artificial Sequence
  e78_d1-1
cgygtkccrggyttytgygayttyacscargaygaycargaaaccgtgcgtttccagtctgcatgactcgaccatccgatttttt

SEQ ID NO: 264 - DNA, 85 bp, Artificial Sequence
  e78_d1-2
rcaraarccyggmacrcgyttsgcraaytcmacratrcgytgmacrccyggsgtmacrcggcatgactcgaccatccgatttttt

SEQ ID NO: 265 - DNA, 85 bp, Artificial Sequence
  e78_d1-3
gyctggarttycaraaratytggctgtggcarcarttyagygcscgygtkacsccrggyggcatgactcgaccatccgatttttt

SEQ ID NO: 266 - DNA, 85 bp, Artificial Sequence
  e78_d1-4
ccaratyttytgraaytccagrctytcsgcmacsgtrctsgcratrccrttytgyggmacgcatgactcgaccatccgatttttt

SEQ ID NO: 267 - DNA, 85 bp, Artificial Sequence
  e78_d1-5
sgargarctgacscgygarctgatgcgycgyccrgtkacsgtkccrcaraayggyatygcgcatgactcgaccatccgatttttt

SEQ ID NO: 268 - DNA, 85 bp, Artificial Sequence
  e78_d1-6
cagytcrcgsgtcagytcytcsgtrtarctrcarttcagrcgrtgsgcytgrctmacrcagcatgactcgaccatccgatttttt

SEQ ID NO: 269 - DNA, 85 bp, Artificial Sequence
  e78_d1-7
ctggcsggyacsgcsaaygarctgacsgtktaygaygtkatyatgtgygtkagycargcsgcatgactcgaccatccgatttttt

SEQ ID NO: 270 - DNA, 85 bp, Artificial Sequence
  e78_d1-8
gcsgtrccsgccagrccrtcrtgrcagtgatgatgatggtggtgcatatgaatcactattgcatgactcgaccatccgatttttt

SEQ ID NO: 271 - DNA, 85 bp, Artificial Sequence
  e78_d2-1
cgyagyctgggygcsaarcayttyagycayctggaytgggaaaccgtgcgtttccagtctgcatgactcgaccatccgatttttt

SEQ ID NO: 272 - DNA, 85 bp, Artificial Sequence
  e78_d2-2
tsgcrcccagrctrcgcagytcyggratyttsgcytccagsgcyggcatcagytgcagsggcatgactcgaccatccgatttttt

SEQ ID NO: 273 - DNA, 85 bp, Artificial Sequence
  e78_d2-3
sctgcgygtkcaratyctgcgyagycgygcsggyagyccrcargcsctgcarctgatgccgcatgactcgaccatccgatttttt

SEQ ID NO: 274 - DNA, 85 bp, Artificial Sequence
  e78_d2-4
agratytgmacrcgcagsgcytcsgcmaccagytcrcgsgcrcgrccratmacyttyggygcatgactcgaccatccgatttttt

SEQ ID NO: 275 - DNA, 85 bp, Artificial Sequence
  e78_d2-5
agygcsatggtkctgctggcsagygaycgygcsggyctgagygarccraargtkatyggygcatgactcgaccatccgatttttt

SEQ ID NO: 276 - DNA, 85 bp, Artificial Sequence
  e78_d2-6
cagmaccatsgcrctraacagrccratytcsgtrtcrctcagrccrtasgcrttcagsgtgcatgactcgaccatccgatttttt

SEQ ID NO: 277 - DNA, 85 bp, Artificial Sequence
  e78_d2-7
gtaygayagygayttygtkaaygcsctgctgaayttygcsaayacsctgaaygcstaygggcatgactcgaccatccgatttttt

SEQ ID NO: 278 - DNA, 85 bp, Artificial Sequence
  e78_d2-8
gcrttmacraartcrctrtcrtacagratytccagytggtgatgatgatggtggtgcatagcatgactcgaccatccgatttttt

SEQ ID NO: 279 - DNA, 85 bp, Artificial Sequence
  e78_d2-9
cttgataacactacatgcatttgtaatcttactcgcacatatgcaccaccatcatcatcagcatgactcgaccatccgatttttt

SEQ ID NO: 280 - DNA, 85 bp, Artificial Sequence
  dhr3_d1-1
aaygcsgtkctgtayggygaygtkatgctgccrcargargaaaccgtgcgtttccagtctgcatgactcgaccatccgatttttt

SEQ ID NO: 281 - DNA, 85 bp, Artificial Sequence
  dhr3_d1-2
rccrtacagmacsgcrttytgrctcagrtccagcagrcgrctcatrcgmacratsgccaggcatgactcgaccatccgatttttt

SEQ ID NO: 282 - DNA, 85 bp, Artificial Sequence
  dhr3_d1-3
cargaygaycaratyctgctgctgaaracsggyagyttygarctggcsatygtkcgyatggcatgactcgaccatccgatttttt

SEQ ID NO: 283 - DNA, 85 bp, Artificial Sequence
  dhr3_d1-4
tcagcagcagratytgrtcrtcytgrctcagrcgcatraarccyggratcagyttsgcragcatgactcgaccatccgatttttt

SEQ ID NO: 284 - DNA, 85 bp, Artificial Sequence
  dhr3_d1-5
gcsgaraarctgacscaratgatycaraayatyatygarttygcsaarctgatyccrggygcatgactcgaccatccgatttttt

SEQ ID NO: 285 - DNA, 85 bp, Artificial Sequence
  dhr3_d1-6
tcatytgsgtcagyttytcsgcrcartccagccacagytcytcytgrcccagrttyttrtgcatgactcgaccatccgatttttt

SEQ ID NO: 286 - DNA, 85 bp, Artificial Sequence
  dhr3_d1-7
tgttycgyaarcarccrgaygtkagycgyatyctgtaytayaaraayctgggycargarggcatgactcgaccatccgatttttt

SEQ ID NO: 287 - DNA, 85 bp, Artificial Sequence
  dhr3_d1-8
yggytgyttrcgraacatrtcrtgmacsgcytccagyttsgtrttsgtrttsgcrtgsgcgcatgactcgaccatccgatttttt

SEQ ID NO: 288 - DNA, 85 bp, Artificial Sequence
  dhr3_d1-9
csgayggygayatyaaygaygtkctgatyaaracsctggcsgargcscaygcsaayacsagcatgactcgaccatccgatttttt

SEQ ID NO: 289 - DNA, 85 bp, Artificial Sequence
  dhr3_d1-10
crttratrtcrccrtcsgcrtgrctratraaytcyggrtcratratsgtgtgatgatgatgcatgactcgaccatccgatttttt

SEQ ID NO: 290 - DNA, 85 bp, Artificial Sequence
  dhr3_d1-11
aaaaacacgataaggcacaacgcatatgcaccaccatcatcatcacacsatyatygayccgcatgactcgaccatccgatttttt

SEQ ID NO: 291 - DNA, 85 bp, Artificial Sequence
  dhr3_d2-1
cayatggaragyctgagyaarttyaarctgcarcayccrgaaaccgtgcgtttccagtctgcatgactcgaccatccgatttttt

SEQ ID NO: 292 - DNA, 85 bp, Artificial Sequence
  dhr3_d2-2
trctcagrctytccatrtgcagratrctratrtcrcgraarttyggratrttrttcagcagcatgactcgaccatccgatttttt

SEQ ID NO: 293 - DNA, 85 bp, Artificial Sequence
  dhr3_d2-3
csccrctgaarggygaygtkacsgtkctggayacsctgctgaayaayatyccraayttycgcatgactcgaccatccgatttttt

SEQ ID NO: 294 - DNA, 85 bp, Artificial Sequence
  dhr3_d2-4
rccyttcagyggsgcrtgrttsgtytccagytcytgrcgratsgcrttcatrctcagrttgcatgactcgaccatccgatttttt

SEQ ID NO: 295 - DNA, 85 bp, Artificial Sequence
  dhr3_d2-5
ayggygtkcgyggyaayacsgaratycarcgyctgttyaayctgagyatgaaygcsatycgcatgactcgaccatccgatttttt

SEQ ID NO: 296 - DNA, 85 bp, Artificial Sequence
  dhr3_d2-6
csgtrttrccrcgmacrccrttrcgytcyggccacagcagmaccagrctytgrtacagsggcatgactcgaccatccgatttttt

SEQ ID NO: 297 - DNA, 85 bp, Artificial Sequence
  dhr3_d2-7
agyatygcsgarctgaarctgacsgaracsgarctggcsctgtaycaragyctggtkctggcatgactcgaccatccgatttttt

SEQ ID NO: 298 - DNA, 85 bp, Artificial Sequence
  dhr3_d2-8
cagyttcagytcsgcratrctyttsgcsgtytgraaratrcgrctmaccagrcgcatytcgcatgactcgaccatccgatttttt

SEQ ID NO: 299 - DNA, 85 bp, Artificial Sequence
  dhr3_d2-9
ggcgcagcgctcatatgcaccaccatcatcatcacagygargaratgcgyctggtkagycgcatgactcgaccatccgatttttt

SEQ ID NO: 300 - DNA, 85 bp, Artificial Sequence
  ecr_d1-1
gaygaraaygaragycaracsgaygtkagyttycgycaygaaaccgtgcgtttccagtctgcatgactcgaccatccgatttttt

SEQ ID NO: 301 - DNA, 85 bp, Artificial Sequence
  ecr_d1-2
sgtytgrctytcrttytcrtcyggytgrctcatratrcgrcgcagrtcytcytcrctygggcatgactcgaccatccgatttttt

SEQ ID NO: 302 - DNA, 85 bp, Artificial Sequence
  ecr_d1-3
tytayaarctgatytggtaycargayggytaygarcarccragygargargayctgcgycgcatgactcgaccatccgatttttt

SEQ ID NO: 303 - DNA, 85 bp, Artificial Sequence
  ecr_d1-4
ccrtcytgrtaccaratcagyttrtaratmacsgccagytgrttrtasgtcagrctyggrgcatgactcgaccatccgatttttt

SEQ ID NO: 304 - DNA, 85 bp, Artificial Sequence
  ecr_d1-5
aygaratyctggcsaartgycargcscgyaayatyccragyctgacstayaaycarctgggcatgactcgaccatccgatttttt

SEQ ID NO: 305 - DNA, 85 bp, Artificial Sequence
  ecr_d1-6
cytgrcayttsgccagratytcrtcyggcagcagyggratsgtsgcrtgytgyggyggytgcatgactcgaccatccgatttttt

SEQ ID NO: 306 - DNA, 85 bp, Artificial Sequence
  ecr_d1-7
ccaccatcatcatcacaaraargaratyctggayctgatgacstgygarccrccrcarcagcatgactcgaccatccgatttttt

SEQ ID NO: 307 - DNA, 85 bp, Artificial Sequence
  ecr_d1-8
yttyttgtgatgatgatggtggtgcatatggtttctagctgcgcagcgcgccagtcgcgagcatgactcgaccatccgatttttt

SEQ ID NO: 308 - DNA, 85 bp, Artificial Sequence
  ecr_d2-1
atyacsgcsggyatygaytgygayagygcsagyacsagygaaaccgtgcgtttccagtctgcatgactcgaccatccgatttttt

SEQ ID NO: 309 - DNA, 85 bp, Artificial Sequence
  ecr_d2-2
tcratrccsgcsgtratsgcrccrccmacrctsgcrcgcatrcgytcsgcrcgytccagrgcatgactcgaccatccgatttttt

SEQ ID NO: 310 - DNA, 85 bp, Artificial Sequence
  ecr_d2-3
agygtkcaragycayctgcaratyacscargargaraaygarcgyctggarcgygcsgargcatgactcgaccatccgatttttt

SEQ ID NO: 311 - DNA, 85 bp, Artificial Sequence
  ecr_d2-4
grtgrctytgmacrctyggyggratsgcrtgmacrtcccaratytcytccagraayttyggcatgactcgaccatccgatttttt

SEQ ID NO: 312 - DNA, 85 bp, Artificial Sequence
  ecr_d2-5
ratgtgyttyagyctgaarctgaaraaycgyaarctgccraarttyctggargaratytggcatgactcgaccatccgatttttt

SEQ ID NO: 313 - DNA, 85 bp, Artificial Sequence
  ecr_d2-6
ttyttcagyttcagrctraarcacatytcsgcrttytgrttrcccagsgtrcgcagytcsgcatgactcgaccatccgatttttt

SEQ ID NO: 314 - DNA, 85 bp, Artificial Sequence
  ecr_d2-7
atcatcatcacgtkttytaygcsaarctgctgagyatyctgacsgarctgcgyacsctgggcatgactcgaccatccgatttttt

SEQ ID NO: 315 - DNA, 85 bp, Artificial Sequence
  ecr_d2-8
sgcrtaraamacgtgatgatgatggtggtgcatatgggctccggaacgcagcatggaagcgcatgactcgaccatccgatttttt

SEQ ID NO: 316 - DNA, 85 bp, Artificial Sequence
  dhr78_d1-1
gtkcarctgtaygcsctgagyagyctgcgycgycarggygaaaccgtgcgtttccagtctgcatgactcgaccatccgatttttt

SEQ ID NO: 317 - DNA, 85 bp, Artificial Sequence
  dhr78_d1-2
ctrctcagsgcrtacagytgmacrcgrcgmacrtarccrcgcagrctrcgytcyttrcgygcatgactcgaccatccgatttttt

SEQ ID NO: 318 - DNA, 85 bp, Artificial Sequence
  dhr78_d1-3
yctgctgcgyctgatyctgctgttyaayccracsctgctgcarcarcgyaargarcgyaggcatgactcgaccatccgatttttt

SEQ ID NO: 319 - DNA, 85 bp, Artificial Sequence
  dhr78_d1-4
gcagratcagrcgcagcagrccraaytccatrtcsgtmacrtccagrctytgcagytcytgcatgactcgaccatccgatttttt

SEQ ID NO: 320 - DNA, 85 bp, Artificial Sequence
  dhr78_d1-5
aratggcsaayctgacscgyacsctgcaygayttygtkcargarctgcaragyctggayggcatgactcgaccatccgatttttt

SEQ ID NO: 321 - DNA, 85 bp, Artificial Sequence
  dhr78_d1-6
trcgsgtcagrttsgccatyttrctratyttcagyggytcratyttrtcratrtcsgccagcatgactcgaccatccgatttttt

SEQ ID NO: 322 - DNA, 85 bp, Artificial Sequence
  dhr78_d1-7
ccatatgcaccaccatcatcatcacacscgycarctggcsgayatygayaaratygarccgcatgactcgaccatccgatttttt

SEQ ID NO: 323 - DNA, 85 bp, Artificial Sequence
  dis_d1-1
agyccrcarcaycgycaratgagycgycayagyctgagygaaaccgtgcgtttccagtctgcatgactcgaccatccgatttttt

SEQ ID NO: 324 - DNA, 85 bp, Artificial Sequence
  dis_d1-2
cgrtgytgyggrctrccrctrctsgtsgtcagsgtyggsgcyggrctratrctrccsgcrgcatgactcgaccatccgatttttt

SEQ ID NO: 325 - DNA, 85 bp, Artificial Sequence
  dis_d1-3
kgaracsgaracsccragyccragyaayagyccrccrctgagygcsggyagyatyagyccgcatgactcgaccatccgatttttt

SEQ ID NO: 326 - DNA, 85 bp, Artificial Sequence
  dis_d1-4
rctyggsgtytcsgtytcmacrttmacrcgyggrctytcrctrcccagrctcagrccytcgcatgactcgaccatccgatttttt

SEQ ID NO: 327 - DNA, 85 bp, Artificial Sequence
  dis_d1-5
tycaragyatyagyagyatyggyagycgyagyggyggyggygargarggyctgagyctgggcatgactcgaccatccgatttttt

SEQ ID NO: 328 - DNA, 85 bp, Artificial Sequence
  dis_d1-6
ratrctrctratrctytgratrctrttrctyggrctyggrctrctrtarttgtgatgatggcatgactcgaccatccgatttttt

SEQ ID NO: 329 - DNA, 85 bp, Artificial Sequence
  dis_d1-7
cctgacaataacgcaccgccagcatatgcaccaccatcatcatcacaaytayagyagyccgcatgactcgaccatccgatttttt

SEQ ID NO: 330 - DNA, 85 bp, Artificial Sequence
  dis_d2-1
gaygargcsacscaracsgaratgaaracsatycargargaaaccgtgcgtttccagtctgcatgactcgaccatccgatttttt

SEQ ID NO: 331 - DNA, 85 bp, Artificial Sequence
  dis_d2-2
sgtytgsgtsgcytcrtcytgcagmacrcgytcrcgratcagyggrctytccagratygggcatgactcgaccatccgatttttt

SEQ ID NO: 332 - DNA, 85 bp, Artificial Sequence
  dis_d2-3
tgctgaayctggcscartggacsatyccrctggayctgacsccratyctggaragyccrcgcatgactcgaccatccgatttttt

SEQ ID NO: 333 - DNA, 85 bp, Artificial Sequence
  dis_d2-4
tgsgccagrttcagcagraacagytcyttccarctytcytgcagcagcagrtgytgrtcrgcatgactcgaccatccgatttttt

SEQ ID NO: 334 - DNA, 85 bp, Artificial Sequence
  dis_d2-5
gytgggtkaartgyctgatgccrttycaracsctgagyaaraaygaycarcayctgctgcgcatgactcgaccatccgatttttt

SEQ ID NO: 335 - DNA, 85 bp, Artificial Sequence
  dis_d2-6
tcagrcayttmacccarcgmacsgccatraacagcagrcgsgcsgtsgtytcytgcagcagcatgactcgaccatccgatttttt

SEQ ID NO: 336 - DNA, 85 bp, Artificial Sequence
  dis_d2-7
catcaccarcarctgctggayagycgyctgctgagytgggaratgctgcargaracsacsgcatgactcgaccatccgatttttt

SEQ ID NO: 337 - DNA, 85 bp, Artificial Sequence
  dis_d2-8
tccagcagytgytggtgatgatgatggtggtgcatatggctactacgggcccttaggaccgcatgactcgaccatccgatttttt

SEQ ID NO: 338 - DNA, 85 bp, Artificial Sequence
  err_d1-1
aaygtkagyctgagyaaygayggygayagyctgaarggygaaaccgtgcgtttccagtctgcatgactcgaccatccgatttttt

SEQ ID NO: 339 - DNA, 85 bp, Artificial Sequence
  err_d1-2
crccrtcrttrctcagrctmacrttrtgcagrtcrcarctcagrctsgtsgtrctrctrcgcatgactcgaccatccgatttttt

SEQ ID NO: 340 - DNA, 85 bp, Artificial Sequence
  err_d1-3
gyctgaaragyagyccragygtkagyccrgarcgycarctgtgyagyagyacsacsagycgcatgactcgaccatccgatttttt

SEQ ID NO: 341 - DNA, 85 bp, Artificial Sequence
  err_d1-4
yggrctrctyttcagrccrttsgtrccrctytgsgtsgcsgtrctyttrctrctyggrctgcatgactcgaccatccgatttttt

SEQ ID NO: 342 - DNA, 85 bp, Artificial Sequence
  err_d1-5
atyaarcargargtkgayacsccragygcsagytgyttyagyccragyagyaaragyacsgcatgactcgaccatccgatttttt

SEQ ID NO: 343 - DNA, 85 bp, Artificial Sequence
  err_d1-6
gsgtrtcmacytcytgyttratrtgcagratrctmacrccrtcrctcatgtgatgatgatgcatgactcgaccatccgatttttt

SEQ ID NO: 344 - DNA, 85 bp, Artificial Sequence
  err_d1-7
agatgcatgttatgaaaagcgccaaacatatgcaccaccatcatcatcacatgagygayggcatgactcgaccatccgatttttt

SEQ ID NO: 345 - DNA, 85 bp, Artificial Sequence
  dhr38_d2-1
aargtkgarcarctgcaratgaaratyatyggyagyctggaaaccgtgcgtttccagtctgcatgactcgaccatccgatttttt

SEQ ID NO: 346 - DNA, 85 bp, Artificial Sequence
  dhr38_d2-2
tytgcagytgytcmacyttyttyggytcrcgcagrccrtgrcgytcsgtratcagsgtcagcatgactcgaccatccgatttttt

SEQ ID NO: 347 - DNA, 85 bp, Artificial Sequence
  dhr38_d2-3
tggaratygayatyagygcsttygcstgyctgtgygcsctgacsctgatyacsgarcgycgcatgactcgaccatccgatttttt

SEQ ID NO: 348 - DNA, 85 bp, Artificial Sequence
  dhr38_d2-4
gcrctratrtcratytccagrttrtgcagrctrcgrctraaytccatratrtcrttcagcgcatgactcgaccatccgatttttt

SEQ ID NO: 349 - DNA, 85 bp, Artificial Sequence
  dhr38_d2-5
kctgcaycgyacscartgyctgcgyagyttyggygartggctgaaygayatyatggarttgcatgactcgaccatccgatttttt

SEQ ID NO: 350 - DNA, 85 bp, Artificial Sequence
  dhr38_d2-6
tgsgtrcgrtgcagmacsgtrccrttrcaraaratcagyttsgtrtcrtcratrcgsgcrgcatgactcgaccatccgatttttt

SEQ ID NO: 351 - DNA, 85 bp, Artificial Sequence
  dhr38_d2-7
aragygcsagyctggarctgttygtkctgcgyctggcstaycgygcscgyatygaygayagcatgactcgaccatccgatttttt

SEQ ID NO: 352 - DNA, 85 bp, Artificial Sequence
  dhr38_d2-8
tccagrctsgcrctytgraacagcagytcytgrtcytcyggcagcagrtcraartarccygcatgactcgaccatccgatttttt

SEQ ID NO: 353 - DNA, 85 bp, Artificial Sequence
  dhr38_d2-9
gyagygtkgaygtkatyaarcarttygcsgaraaratyccrggytayttygayctgctgcgcatgactcgaccatccgatttttt

SEQ ID NO: 354 - DNA, 85 bp, Artificial Sequence
  dhr38_d2-10
tratmacrtcmacrctrctsgtcagcagytgrtaraaytgytgmacyttrtcsgcytcrcgcatgactcgaccatccgatttttt

SEQ ID NO: 355 - DNA, 85 bp, Artificial Sequence
  dhr38_d2-11
gatgacgctacatatgcaccaccatcatcatcacagyatgagygargcsgayaargtkcagcatgactcgaccatccgatttttt

SEQ ID NO: 356 - DNA, 85 bp, Artificial Sequence
  ftz_d1-1
gcsagyacsggyggygtkatygcsacsccratgaaygcsgaaaccgtgcgtttccagtctgcatgactcgaccatccgatttttt

SEQ ID NO: 357 - DNA, 85 bp, Artificial Sequence
  ftz_d1-2
crccsgtrctsgcrttmacytgrcccagsgcratsgcratyggrctyggrctrctrtcyggcatgactcgaccatccgatttttt

SEQ ID NO: 358 - DNA, 85 bp, Artificial Sequence
  ftz_d1-3
arcargaratycaratyccrcargtkagyagyctgacscaragyccrgayagyagyccragcatgactcgaccatccgatttttt

SEQ ID NO: 359 - DNA, 85 bp, Artificial Sequence
  ftz_d1-4
cytgyggratytgratytcytgyttratrttcatrttyggrtasgcytgytgrtarccyggcatgactcgaccatccgatttttt

SEQ ID NO: 360 - DNA, 85 bp, Artificial Sequence
  ftz_d1-5
cgyaayagyatgggyccrgayatyaarccracsccratyagyccrggytaycarcargcsgcatgactcgaccatccgatttttt

SEQ ID NO: 361 - DNA, 85 bp, Artificial Sequence
  ftz_d1-6
ggrcccatrctrttrcgcagsgcytgcagsgccagytgrcgytgrcgcatmacytgcagygcatgactcgaccatccgatttttt

SEQ ID NO: 362 - DNA, 85 bp, Artificial Sequence
  ftz_d1-7
gyaayaarttyggyccratgtayaarcgygaycgygcscgyaarctgcargtkatgcgycgcatgactcgaccatccgatttttt

SEQ ID NO: 363 - DNA, 85 bp, Artificial Sequence
  ftz_d1-8
tyggrccraayttrttrcggtgatgatgatggtggtgcatatgtctcctatccacattttgcatgactcgaccatccgatttttt

SEQ ID NO: 364 - DNA, 85 bp, Artificial Sequence
  DHR39_d1-1
tgggarggygarctgagygayacsgargtkaayggyggygaaaccgtgcgtttccagtctgcatgactcgaccatccgatttttt

SEQ ID NO: 365 - DNA, 85 bp, Artificial Sequence
  DHR39_d1-2
cagytcrccytcccarctcatyggrcgyggrccrttyttrtcsgcsgcsgtrcgrcgrcggcatgactcgaccatccgatttttt

SEQ ID NO: 366 - DNA, 85 bp, Artificial Sequence
  DHR39_d1-3
ragygayagygargargarctggcsagyatygaraayctgaargtkcgycgycgyacsgcgcatgactcgaccatccgatttttt

SEQ ID NO: 367 - DNA, 85 bp, Artificial Sequence
  DHR39_d1-4
tcytcytcrctrtcrctytcrcgrtgrttrctrtgrccrctcagyggratrcgcagrctrgcatgactcgaccatccgatttttt

SEQ ID NO: 368 - DNA, 85 bp, Artificial Sequence
  DHR39_d1-5
aayggyaayatggtkccrgtkatygcsaaytaygtkcayggyagyctgcgyatyccrctggcatgactcgaccatccgatttttt

SEQ ID NO: 369 - DNA, 85 bp, Artificial Sequence
  DHR39_d1-6
ggmaccatrttrccrttyggrctyggcagytcrtccagytcrcayttratrttsgtmacrgcatgactcgaccatccgatttttt

SEQ ID NO: 370 - DNA, 85 bp, Artificial Sequence
  DHR39_d1-7
gyagygcsggyggygcsacsggyagycgycayaaygtkagygtkacsaayatyaartgyggcatgactcgaccatccgatttttt

SEQ ID NO: 371 - DNA, 85 bp, Artificial Sequence
  DHR39_d1-8
crccsgcrctrctrcccagsgtsgtsgtsgtrttsgcmacsgtsgtsgtrcacatrttmagcatgactcgaccatccgatttttt

SEQ ID NO: 372 - DNA, 85 bp, Artificial Sequence
  DHR39_d1-9
ragyggyccrctgggyggyagyagyggytaycargtkccrgtkaayatgtgyacsacsacgcatgactcgaccatccgatttttt

SEQ ID NO: 373 - DNA, 85 bp, Artificial Sequence
  DHR39_d1-10
ccrcccagyggrccrctytgytgytcsgcyttratrctrctcatrttyggcatgtgatgagcatgactcgaccatccgatttttt

SEQ ID NO: 374 - DNA, 85 bp, Artificial Sequence
  DHR39_d1-11
cggattggtagttgtgccgaaccatatgcaccaccatcatcatcacatgccraayatgaggcatgactcgaccatccgatttttt

SEQ ID NO: 375 - DNA, 85 bp, Artificial Sequence
  dhr39_d2-1
acsgaygcsgarctggcscgyatyaaycarccrctgagygaaaccgtgcgtttccagtctgcatgactcgaccatccgatttttt

SEQ ID NO: 376 - DNA, 85 bp, Artificial Sequence
  dhr39_d2-2
gccagytcsgcrtcsgtrtaytgccacagrtgytcmacrtccatratytcytgcagcagygcatgactcgaccatccgatttttt

SEQ ID NO: 377 - DNA, 85 bp, Artificial Sequence
  dhr39_d2-3
sggyaarcaragyctgcgyacsggyagygtkccrccrctgctgcargaratyatggaygtgcatgactcgaccatccgatttttt

SEQ ID NO: 378 - DNA, 85 bp, Artificial Sequence
  dhr39_d2-4
gcagrctytgyttrccsgtytcsgccatytcytcrctyttmacrctsgtrccsgccagrcgcatgactcgaccatccgatttttt

SEQ ID NO: 379 - DNA, 85 bp, Artificial Sequence
  dhr39_d2-5
gtkccratyccrtgyagyacsagyctgccrgcsagyccragyctggcsggyacsagygtkgcatgactcgaccatccgatttttt

SEQ ID NO: 380 - DNA, 85 bp, Artificial Sequence
  dhr39_d2-6
rctsgtrctrcayggratyggmacrccrccraarccrttcagytgrtgcagrcgytgrtggcatgactcgaccatccgatttttt

SEQ ID NO: 381 - DNA, 85 bp, Artificial Sequence
  dhr39_d2-7
accaccatcatcatcacgcsagycarcarcarccrcaycarcgyctgcaycarctgaayggcatgactcgaccatccgatttttt

SEQ ID NO: 382 - DNA, 85 bp, Artificial Sequence
  dhr39_d2-8
gcgtgatgatgatggtggtgcatatgccataaggaccactgaggttctcgtgaaggttaagcatgactcgaccatccgatttttt

SEQ ID NO: 383 - DNA, 85 bp, Artificial Sequence
  dhr4_d1-1
acsgarcayctgctgagycaragyatgcarcayctgacsgaaaccgtgcgtttccagtctgcatgactcgaccatccgatttttt

SEQ ID NO: 384 - DNA, 85 bp, Artificial Sequence
  dhr4_d1-2
ytgrctcagcagrtgytcsgtyggyggrctrctyttrcgcagsgtcagyggytgsgtcaggcatgactcgaccatccgatttttt

SEQ ID NO: 385 - DNA, 85 bp, Artificial Sequence
  dhr4_d1-3
ctgggyccraayatygtkcaracscaycayctgcaycarcarctgacscarccrctgacsgcatgactcgaccatccgatttttt

SEQ ID NO: 386 - DNA, 85 bp, Artificial Sequence
  dhr4_d1-4
gmacratrttyggrcccagrctrccrtgrctcagytgsgtyggyggrctsgcrctmacrcgcatgactcgaccatccgatttttt

SEQ ID NO: 387 - DNA, 85 bp, Artificial Sequence
  dhr4_d1-5
ygarcgygarcaragyatyagyagyagycarcarcayctgagycgygtkagygcsagyccgcatgactcgaccatccgatttttt

SEQ ID NO: 388 - DNA, 85 bp, Artificial Sequence
  dhr4_d1-6
ratrctytgytcrcgytcrcgytcrcgrtcrcgrtcrcgytcgtgatgatgatggtggtggcatgactcgaccatccgatttttt

SEQ ID NO: 389 - DNA, 85 bp, Artificial Sequence
  dhr4_d1-7
acacgtctgtcggtccgtaggttacgcggacggacatatgcaccaccatcatcatcacgagcatgactcgaccatccgatttttt

SEQ ID NO: 390 - DNA, 85 bp, Artificial Sequence
  dhr4_d2-1
agyaaratgctggtkttycaycarcarcgygarcargargaaaccgtgcgtttccagtctgcatgactcgaccatccgatttttt

SEQ ID NO: 391 - DNA, 85 bp, Artificial Sequence
  dhr4_d2-2
tgraamaccagcatyttrctrctmacrttcagcagrcgytcytcyttmacrctrcgratmgcatgactcgaccatccgatttttt

SEQ ID NO: 392 - DNA, 85 bp, Artificial Sequence
  dhr4_d2-3
csggyagyagygcsaarctgagygargcsggyatgagygtkatycgyagygtkaargarggcatgactcgaccatccgatttttt

SEQ ID NO: 393 - DNA, 85 bp, Artificial Sequence
  dhr4_d2-4
ttsgcrctrctrccsgtrctratrccsgtrccsgcrctsgcrctrctcagmacrccrttsgcatgactcgaccatccgatttttt

SEQ ID NO: 394 - DNA, 85 bp, Artificial Sequence
  dhr4_d2-5
agyacsacsacsacsacsggycgyccracsctgacsccracsaayggygtkctgagyagygcatgactcgaccatccgatttttt

SEQ ID NO: 395 - DNA, 85 bp, Artificial Sequence
  dhr4_d2-6
rccsgtsgtsgtsgtsgtrctyggyttytcytccagrctrtcyggrctsgcrcgrctgtggcatgactcgaccatccgatttttt

SEQ ID NO: 396 - DNA, 85 bp, Artificial Sequence
  dhr4_d2-7
ctaaataattgacatcgctccatatgcaccaccatcatcatcacagycgygcsagyccrggcatgactcgaccatccgatttttt

SEQ ID NO: 397 - DNA, 85 bp, Artificial Sequence
  brc_d1-1
carctgctgaarcgyatggcsatgatgcaycgyagyagygaaaccgtgcgtttccagtctgcatgactcgaccatccgatttttt

SEQ ID NO: 398 - DNA, 85 bp, Artificial Sequence
  brc_d1-2
trcgyttcagcagytgrttrttratrtgrctyggcagrctyggmacsgcsgtyggyggyggcatgactcgaccatccgatttttt

SEQ ID NO: 399 - DNA, 85 bp, Artificial Sequence
  brc_d1-3
ygayggyggyagyagyacsctgttyagycgycarggygcsggyagyccrccrccracsgcgcatgactcgaccatccgatttttt

SEQ ID NO: 400 - DNA, 85 bp, Artificial Sequence
  brc_d1-4
rctrccrccrtcrtcrtgcagrctrccrtgrtgyggrtgyggcagrctytgsgtrtgsgtgcatgactcgaccatccgatttttt

SEQ ID NO: 401 - DNA, 85 bp, Artificial Sequence
  brc_d1-5
aratycaraayctggcsaayagyggyggycgyacsccrctgaayacscayacscaragycgcatgactcgaccatccgatttttt

SEQ ID NO: 402 - DNA, 85 bp, Artificial Sequence
  brc_d1-6
rctrttsgccagrttytgratytgsgccagrtgrctrtgsgtrtcytcsgcgtgatgatggcatgactcgaccatccgatttttt

SEQ ID NO: 403 - DNA, 85 bp, Artificial Sequence
  brc_d1-7
gacgatggggcggcttgatgtaagtcatatgcaccaccatcatcatcacgcsgargayacgcatgactcgaccatccgatttttt

SEQ ID NO: 404 - DNA, 85 bp, Artificial Sequence
  brc_d2-1
acscarctgcarcaragyggygayctggcsgtkagyccrgaaaccgtgcgtttccagtctgcatgactcgaccatccgatttttt

SEQ ID NO: 405 - DNA, 85 bp, Artificial Sequence
  brc_d2-2
crctytgytgcagytgsgtrttcagrcccagcagsgcrctyggrtcratrttyggraartgcatgactcgaccatccgatttttt

SEQ ID NO: 406 - DNA, 85 bp, Artificial Sequence
  brc_d2-3
ccrgcsgaraayaaratgttycaygcsgcsgcsttyaayttyccraayatygayccragygcatgactcgaccatccgatttttt

SEQ ID NO: 407 - DNA, 85 bp, Artificial Sequence
  brc_d2-4
catyttrttytcsgcyggrctcatratraaytgyggsgcsgcrtgrtgrctsgccagrccgcatgactcgaccatccgatttttt

SEQ ID NO: 408 - DNA, 85 bp, Artificial Sequence
  brc_d2-5
yaarggyagyctgagyagyggyaaygaygargaratyggygayggyctggcsagycaycagcatgactcgaccatccgatttttt

SEQ ID NO: 409 - DNA, 85 bp, Artificial Sequence
  brc_d2-6
rctrctcagrctrccyttrccrccrtcrccrctrctrctrcgrttsgcrtcrtgytcrccgcatgactcgaccatccgatttttt

SEQ ID NO: 410 - DNA, 85 bp, Artificial Sequence
  brc_d2-7
catcatcacgcsaaygcsaaygaygarcayagyaaygayagyacsggygarcaygaygcsgcatgactcgaccatccgatttttt

SEQ ID NO: 411 - DNA, 85 bp, Artificial Sequence
  brc_d2-8
sgcrttsgcgtgatgatgatggtggtgcatatgattagaagcggaagcggggtatcgccagcatgactcgaccatccgatttttt

SEQ ID NO: 412 - DNA, 85 bp, Artificial Sequence
  e74_d1-1
aaycgygaraarggygtkttyaarctggtkgayagyaargaaaccgtgcgtttccagtctgcatgactcgaccatccgatttttt

SEQ ID NO: 413 - DNA, 85 bp, Artificial Sequence
  e74_d1-2
crccyttytcrcgrttsgtccayttratraarcgyggrcartaytcrcgrtcytgcagcagcatgactcgaccatccgatttttt

SEQ ID NO: 414 - DNA, 85 bp, Artificial Sequence
  e74_d1-3
ycgygarggyagyacsacstayctgtgggarttyctgctgaarctgctgcargaycgygagcatgactcgaccatccgatttttt

SEQ ID NO: 415 - DNA, 85 bp, Artificial Sequence
  e74_d1-4
sgtrctrccytcrcgrctrcgrcgyttmacrcccatytccagyttyggyttrcgyggyttgcatgactcgaccatccgatttttt

SEQ ID NO: 416 - DNA, 85 bp, Artificial Sequence
  e74_d1-5
agytayatgctggarctgggyggyttycarcarcgyaargcsaaraarccrcgyaarccrgcatgactcgaccatccgatttttt

SEQ ID NO: 417 - DNA, 85 bp, Artificial Sequence
  e74_d1-6
ccagytccagcatrtarctcagrtcrtarctmacgtgatgatgatggtggtgcatatgctgcatgactcgaccatccgatttttt

SEQ ID NO: 418 - DNA, 85 bp, Artificial Sequence
  e74_d2-1
gaygtkccraargayatyatygaratygaytgyaayggygaaaccgtgcgtttccagtctgcatgactcgaccatccgatttttt

SEQ ID NO: 419 - DNA, 85 bp, Artificial Sequence
  e74_d2-2
tratrtcyttyggmacrtcmacraaytgrtamaccagrcgytgrccrtcmacyttsgccagcatgactcgaccatccgatttttt

SEQ ID NO: 420 - DNA, 85 bp, Artificial Sequence
  e74_d2-3
racsatgggycgygcsctgcgytaytaytaycarcgyggyatyctggcsaargtkgaygggcatgactcgaccatccgatttttt

SEQ ID NO: 421 - DNA, 85 bp, Artificial Sequence
  e74_d2-4
gcrcgrcccatsgtytcrtarttcatrtcyggyttrttyttrtgcatrccccacagrcgrgcatgactcgaccatccgatttttt

SEQ ID NO: 422 - DNA, 85 bp, Artificial Sequence
  e74_d2-5
aaycgygaraarggygtkttyaarctggtkgayagyaargcsgtkagycgyctgtggggygcatgactcgaccatccgatttttt

SEQ ID NO: 423 - DNA, 85 bp, Artificial Sequence; identical to SEQ ID NO: 413
  e74_d2-6

SEQ ID NO: 424 - DNA, 85 bp, Artificial Sequence
  e74_d2-7
ccatcatcatcacacsacstayctgtgggarttyctgctgaarctgctgcargaycgygagcatgactcgaccatccgatttttt

SEQ ID NO: 425 - DNA, 85 bp, Artificial Sequence
  e74_d2-8
grtasgtsgtgtgatgatgatggtggtgcatatgtcacgacgagccaccctcattcttgcgcatgactcgaccatccgatttttt

SEQ ID NO: 426 - DNA, 85 bp, Artificial Sequence
  e93_d1-1
gtkgaraaygtktaygayggyatyatycgyaaracsctggaaaccgtgcgtttccagtctgcatgactcgaccatccgatttttt

SEQ ID NO: 427 - DNA, 85 bp, Artificial Sequence
  e93_d1-2
rccrtcrtamacrttytcmaccatrtcytgsgcrctyttcatcagrccrtgrtgrttrctgcatgactcgaccatccgatttttt

SEQ ID NO: 428 - DNA, 85 bp, Artificial Sequence
  e93_d1-3
tggayttyaaycgyatyacsgargcsatgcgyaayccrcargcsagyaaycaycayggycgcatgactcgaccatccgatttttt

SEQ ID NO: 429 - DNA, 85 bp, Artificial Sequence
  e93_d1-4
sgtratrcgrttraartccagrccrtasgcrttsgtsgcrttsgtytgyggccaraacatgcatgactcgaccatccgatttttt

SEQ ID NO: 430 - DNA, 85 bp, Artificial Sequence
  e93_d1-5
ctgttygargcsggyccrcargcsctgagyttycarccraayatgttytggccrcaracsgcatgactcgaccatccgatttttt

SEQ ID NO: 431 - DNA, 85 bp, Artificial Sequence
  e93_d1-6
ggrccsgcytcraacagyggcagyttcagrccrttyggsgtgtgatgatgatggtggtgcgcatgactcgaccatccgatttttt

SEQ ID NO: 432 - DNA, 85 bp, Artificial Sequence
  e93_d1-7
tggcgttgccaggccttattgtgtctgtctctgtgcatatgcaccaccatcatcatcacagcatgactcgaccatccgatttttt

SEQ ID NO: 433 - DNA, 85 bp, Artificial Sequence
  e93_d2-1
gayttyatyaarggyctgctggtkgcsaayagyggyggygaaaccgtgcgtttccagtctgcatgactcgaccatccgatttttt

SEQ ID NO: 434 - DNA, 85 bp, Artificial Sequence
  e93_d2-2
accagcagrccyttratraartcrccmacsgtytgsgtytcrtccagyttyggcatsgcrgcatgactcgaccatccgatttttt

SEQ ID NO: 435 - DNA, 85 bp, Artificial Sequence
  e93_d2-3
csaaygtkctgctgcayacsctgatgctggcsgcsggyatyggygcsatgccraarctgggcatgactcgaccatccgatttttt

SEQ ID NO: 436 - DNA, 85 bp, Artificial Sequence
  e93_d2-4
trtgcagcagmacrttsgcrtccagyggratrctyggyggsgcrccytgrttsgcrctsggcatgactcgaccatccgatttttt

SEQ ID NO: 437 - DNA, 85 bp, Artificial Sequence
  e93_d2-5
cgyggyagyccraaratgggycgycayccrgcstgyggyaaygcsagygcsaaycarggygcatgactcgaccatccgatttttt

SEQ ID NO: 438 - DNA, 85 bp, Artificial Sequence
  e93_d2-6
tyttyggrctrccrcgrtcmacrtcytcrcccagrtcrctrccrttrtgytcrctcagrtgcatgactcgaccatccgatttttt

SEQ ID NO: 439 - DNA, 85 bp, Artificial Sequence
  e93_d2-7
ccaccatcatcatcaccgygcscarctgcgyaarctgagycayctgagygarcayaaygggcatgactcgaccatccgatttttt

SEQ ID NO: 440 - DNA, 85 bp, Artificial Sequence
  e93_d2-8
crcggtgatgatgatggtggtgcatatggggatgcggcacgtgagtgagtacataagggcgcatgactcgaccatccgatttttt

SEQ ID NO: 441 - DNA, 85 bp, Artificial Sequence
  mld_d1-1
aaracscayctgccrctgggygtkttycgyaaygargaygaaaccgtgcgtttccagtctgcatgactcgaccatccgatttttt

SEQ ID NO: 442 - DNA, 85 bp, Artificial Sequence
  mld_d1-2
cagyggcagrtgsgtyttcatrtgsgcrccrtgraayttratrtgsgtyttcagsgcrctgcatgactcgaccatccgatttttt

SEQ ID NO: 443 - DNA, 85 bp, Artificial Sequence
  mld_d1-3
arccrcayacstgygaygartgyggyaarcarttyggyacsgaragygcsctgaaracscgcatgactcgaccatccgatttttt

SEQ ID NO: 444 - DNA, 85 bp, Artificial Sequence
  mld_d1-4
crcasgtrtgyggyttrctcagcagrtgmacsgtratyttrtgcagsgtcagytgrctyggcatgactcgaccatccgatttttt

SEQ ID NO: 445 - DNA, 85 bp, Artificial Sequence
  mld_d1-5
gyccrtaygartgyaayatytgycgygtkcgyttyccrcgyccragycarctgacsctgcgcatgactcgaccatccgatttttt

SEQ ID NO: 446 - DNA, 85 bp, Artificial Sequence
  mld_d1-6
trttrcaytcrtayggrcgrccytcmacrtcrtgrctytccatrtgrcgrtgcagsgcsggcatgactcgaccatccgatttttt

SEQ ID NO: 447 - DNA, 85 bp, Artificial Sequence
  mld_d1-7
satgccrtaygtktgyacsatytgyaarcgyggytaycgyatgcgyacsgcsctgcaycggcatgactcgaccatccgatttttt

SEQ ID NO: 448 - DNA, 85 bp, Artificial Sequence
  mld_d1-8
ratsgtrcamacrtayggcatsgtsgtcagsgcrccrtgsgccagytgrtgrtgyttyttgcatgactcgaccatccgatttttt

SEQ ID NO: 449 - DNA, 85 bp, Artificial Sequence
  mld_d1-9
yctgtaytgygargarcgyttyacsaaygaratyagyctgaaraarcaycaycarctggcgcatgactcgaccatccgatttttt

SEQ ID NO: 450 - DNA, 85 bp, Artificial Sequence
  mld_d1-10
rcgytcytcrcartacagrcartgrtgrctsgtrcgcagytcrctmacytggtgatgatggcatgactcgaccatccgatttttt

SEQ ID NO: 451 - DNA, 85 bp, Artificial Sequence
  mld_d1-11
ttccactgtttgtctctatcgtcgccatatgcaccaccatcatcatcaccargtkagygagcatgactcgaccatccgatttttt

SEQ ID NO: 452 - DNA, 85 bp, Artificial Sequence
  salm/salr_d1-1
acsggygarccracsgayctgacsccrgarcaratycargaaaccgtgcgtttccagtctgcatgactcgaccatccgatttttt

SEQ ID NO: 453 - DNA, 85 bp, Artificial Sequence
  salm/salr_d1-2
tcsgtyggytcrccsgtrtgcagrcgratrtgytgytgcagmaccagsgcrttrctrtaygcatgactcgaccatccgatttttt

SEQ ID NO: 454 - DNA, 85 bp, Artificial Sequence
  salm/salr_d1-3
cratgcgyaayttycaycartgyccrgtktgycayaaraartayagyaaygcsctggtkcgcatgactcgaccatccgatttttt

SEQ ID NO: 455 - DNA, 85 bp, Artificial Sequence
  salm/salr_d1-4
caytgrtgraarttrcgcatyggyggrcgratyttrtgmacsgccatrtgsgtyttcagrgcatgactcgaccatccgatttttt

SEQ ID NO: 456 - DNA, 85 bp, Artificial Sequence
  salm/salr_d1-5
artgycgyatytgyggycgygcsttyacsacsaarggyaayctgaaracscayatggcsggcatgactcgaccatccgatttttt

SEQ ID NO: 457 - DNA, 85 bp, Artificial Sequence
  salm/salr_d1-6
rccrcaratrcgrcayttraayggrcgytcrccsgtrtgsgtrcgrtartgcatytgcaggcatgactcgaccatccgatttttt

SEQ ID NO: 458 - DNA, 85 bp, Artificial Sequence
  salm/salr_d1-7
tgygtkgtktgygaycgygtkctgagytgyaaragygcsctgcaratgcaytaycgyacsgcatgactcgaccatccgatttttt

SEQ ID NO: 459 - DNA, 85 bp, Artificial Sequence
  salm/salr_d1-8
crcgrtcrcamacmacrcaytgrttyggrtcrctratyttyttrttyttcatcagytcytgcatgactcgaccatccgatttttt

SEQ ID NO: 460 - DNA, 85 bp, Artificial Sequence
  salm/salr_d1-9
tygargtkagyaayacstgygaracsatgaarctgaargarctgatgaaraayaaraaragcatgactcgaccatccgatttttt

SEQ ID NO: 461 - DNA, 85 bp, Artificial Sequence
  salm/salr_d1-10
rcasgtrttrctmacytcratraarttytcccarctrttrtcrttrctrtgyggrcgygggcatgactcgaccatccgatttttt

SEQ ID NO: 462 - DNA, 85 bp, Artificial Sequence
  salm/salr_d1-11
tcatcatcacttyttyaayccratyaarcaygaratggcsgcsctgctgccrcgyccrcagcatgactcgaccatccgatttttt

SEQ ID NO: 463 - DNA, 85 bp, Artificial Sequence
  salm/salr_d1-12
tyggrttraaraagtgatgatgatggtggtgcatatgagagcgcaagactgccttgaaaagcatgactcgaccatccgatttttt

SEQ ID NO: 464 - DNA, 85 bp, Artificial Sequence
  salm/salr_d2-1
cgygcsgtkaartayatgagygartggaaygargaycgygaaaccgtgcgtttccagtctgcatgactcgaccatccgatttttt

SEQ ID NO: 465 - DNA, 85 bp, Artificial Sequence
  salm/salr_d2-2
caytcrctcatrtayttmacsgcrcgrttrcgraasgtytcytgytccatrtcrcgratygcatgactcgaccatccgatttttt

SEQ ID NO: 466 - DNA, 85 bp, Artificial Sequence
  salm/salr_d2-3
yacsacsaarggyaayctgaarcarcayatgctgacscayaaratycgygayatggarcagcatgactcgaccatccgatttttt

SEQ ID NO: 467 - DNA, 85 bp, Artificial Sequence
  salm/salr_d2-4
cagrttrccyttsgtsgtraarccrcgrtcrcaratrctrcayttraayggrcgytcyttgcatgactcgaccatccgatttttt

SEQ ID NO: 468 - DNA, 85 bp, Artificial Sequence
  salm/salr_d2-5
rtgycayagygcsctggaratycaytaycgyagycayacsaargarcgyccrttyaartggcatgactcgaccatccgatttttt

SEQ ID NO: 469 - DNA, 85 bp, Artificial Sequence
  salm/salr_d2-6
gsgcrctrtgrcayggraasgtyttrtarcaratrccrcasgtsgtrctrccrcgmacrcgcatgactcgaccatccgatttttt

SEQ ID NO: 470 - DNA, 85 bp, Artificial Sequence
  salm/salr_d2-7
aragygtkatgccrgcsgcsccrttyaayccrctggcsctgagyggygtkcgyggyagyagcatgactcgaccatccgatttttt

SEQ ID NO: 471 - DNA, 85 bp, Artificial Sequence
  salm/salr_d2-8
sgcyggcatmacrctytgsgcratytgrttcatsgcrttrcagtgatgatgatggtggtggcatgactcgaccatccgatttttt

SEQ ID NO: 472 - DNA, 85 bp, Artificial Sequence
  salm/salr_d2-9
tcatgagggttaaagaggctcgcagttaggaaacatatgcaccaccatcatcatcactgygcatgactcgaccatccgatttttt

SEQ ID NO: 473 - DNA, 85 bp, Artificial Sequence
  ac_d1-1
ctgaaratggcsgtkgartayatycgycgyctgcaraargaaaccgtgcgtttccagtctgcatgactcgaccatccgatttttt

SEQ ID NO: 474 - DNA, 85 bp, Artificial Sequence
  ac_d1-2
rcgratrtaytcmacsgccatyttcagsgtrctmacyttrctcagyttyttrttsgcrccgcatgactcgaccatccgatttttt

SEQ ID NO: 475 - DNA, 85 bp, Artificial Sequence
  ac_d1-3
atygcsgayctgagyaayggycgycgyggyatyggyccrggygcsaayaaraarctgagygcatgactcgaccatccgatttttt

SEQ ID NO: 476 - DNA, 85 bp, Artificial Sequence
  ac_d1-4
cgrccrttrctcagrtcsgcratmacsgcsgcyggratrtgytgrcgcagytgrctraargcatgactcgaccatccgatttttt

SEQ ID NO: 477 - DNA, 85 bp, Artificial Sequence
  ac_d1-5
cgygarcgyaaycgygtkaarcargtkaayaayggyttyagycarctgcgycarcayatygcatgactcgaccatccgatttttt

SEQ ID NO: 478 - DNA, 85 bp, Artificial Sequence
  ac_d1-6
rcgrttrcgytcrcgsgcrttrcgrcgratmacrctyggrccgtgatgatgatggtggtggcatgactcgaccatccgatttttt

SEQ ID NO: 479 - DNA, 85 bp, Artificial Sequence
  ac_d1-7
ccgtcatgggaagtatttagtttctcctccactccatatgcaccaccatcatcatcacgggcatgactcgaccatccgatttttt

SEQ ID NO: 480 - DNA, 85 bp, Artificial Sequence
  ac_d2-1
gargayatyctggaytayatyagyctgtggcargaygaygaaaccgtgcgtttccagtctgcatgactcgaccatccgatttttt

SEQ ID NO: 481 - DNA, 85 bp, Artificial Sequence
  ac_d2-2
cacagrctratrtartccagratrtcytcrtcytcsgtrccrctrctrcarctrttrttrgcatgactcgaccatccgatttttt

SEQ ID NO: 482 - DNA, 85 bp, Artificial Sequence
  ac_d2-3
ayacsaarctggargcsagyttygargaytaycgyaayaayagytgyagyagyggyacsggcatgactcgaccatccgatttttt

SEQ ID NO: 483 - DNA, 85 bp, Artificial Sequence
  ac_d2-4
craarctsgcytccagyttsgtrtgraarttrttyggyggsgtsgcrccyggratsgtrcgcatgactcgaccatccgatttttt

SEQ ID NO: 484 - DNA, 85 bp, Artificial Sequence
  ac_d2-5
sagyagytgyaayagyatyagyagytaytgyaarccrgcsacsagyacsatyccrggygcgcatgactcgaccatccgatttttt

SEQ ID NO: 485 - DNA, 85 bp, Artificial Sequence
  ac_d2-6
rctratrctrttrcarctrctsgtrctrccsgtyggrctytgcagytgcagytcytgrtggcatgactcgaccatccgatttttt

SEQ ID NO: 486 - DNA, 85 bp, Artificial Sequence
  ac_d2-7
yttycarcarcarcarcarcaycarcayctgtaygcstggcaycargarctgcarctgcagcatgactcgaccatccgatttttt

SEQ ID NO: 487 - DNA, 85 bp, Artificial Sequence
  ac_d2-8
gytgytgytgytgytgraartgcagrtgytgytgytgcagrtgcagytgyttytgyttytgcatgactcgaccatccgatttttt

SEQ ID NO: 488 - DNA, 85 bp, Artificial Sequence
  ac_d2-9
atatgcaccaccatcatcatcacgaraaygaycarcaraarcaraarcarctgcayctgcgcatgactcgaccatccgatttttt

SEQ ID NO: 489 - DNA, 85 bp, Artificial Sequence
  ac_d2-10
tgatgatgatggtggtgcatatggtcgacgatgcgtgacagggtagacaacgaaccaatcgcatgactcgaccatccgatttttt

SEQ ID NO: 490 - DNA, 85 bp, Artificial Sequence
  sc_d1-1
ggyggyagyaayatyggygcsaayaaygcsgtkacscargaaaccgtgcgtttccagtctgcatgactcgaccatccgatttttt

SEQ ID NO: 491 - DNA, 85 bp, Artificial Sequence
  sc_d1-2
rccratrttrctrccrccrttcagrtcrtcmaccagrtcytgcagrcgrcgratrtaytcgcatgactcgaccatccgatttttt

SEQ ID NO: 492 - DNA, 85 bp, Artificial Sequence
  sc_d1-3
yaaraaratyagyaargtkgayacsctgcgyatygcsgtkgartayatycgycgyctgcagcatgactcgaccatccgatttttt

SEQ ID NO: 493 - DNA, 85 bp, Artificial Sequence
  sc_d1-4
cagsgtrtcmacyttrctratyttyttrtgyggrccrcgrccrccrccyttsgtcagrtcgcatgactcgaccatccgatttttt

SEQ ID NO: 494 - DNA, 85 bp, Artificial Sequence
  sc_d1-5
ygcscgyctgcgycarcayatyccrcaragyatyatyacsgayctgacsaarggyggygggcatgactcgaccatccgatttttt

SEQ ID NO: 495 - DNA, 85 bp, Artificial Sequence
  sc_d1-6
grcgcagrcgsgcraarctrttrttmacytgyttmacrcgrttrcgytcrcgsgcrttrcgcatgactcgaccatccgatttttt

SEQ ID NO: 496 - DNA, 85 bp, Artificial Sequence
  sc_d1-7
sccrtayaaygtkgaycaragycaragygtkcarcgycgyaaygcscgygarcgyaaycggcatgactcgaccatccgatttttt

SEQ ID NO: 497 - DNA, 85 bp, Artificial Sequence
  sc_d1-8
tytgrctytgrtcmacrttrtayggsgcgtgatgatgatggtggtgcatatgtcgcacatgcatgactcgaccatccgatttttt

SEQ ID NO: 498 - DNA, 85 bp, Artificial Sequence
  l(1)sc_d1-1
atycgyggyctgcargayatgctggaygayggyacsgcsgaaaccgtgcgtttccagtctgcatgactcgaccatccgatttttt

SEQ ID NO: 499 - DNA, 85 bp, Artificial Sequence
  l(1)sc_d1-2
gcatrtcytgcagrccrcgratrtaytcmacsgcratrcgcagsgtrtcmacyttrctcagcatgactcgaccatccgatttttt

SEQ ID NO: 500 - DNA, 85 bp, Artificial Sequence
  l(1)sc_d1-3
ayagyctgagyaayggyggycgyggyagyagyaaraarctgagyaargtkgayacsctgcgcatgactcgaccatccgatttttt

SEQ ID NO: 501 - DNA, 85 bp, Artificial Sequence
  l(1)sc_d1-4
ccrccrttrctcagrctrttmacmacsgtytgyggcagrtgytgrcgcagrttmacraargcatgactcgaccatccgatttttt

SEQ ID NO: 502 - DNA, 85 bp, Artificial Sequence
  l(1)sc_d1-5
yaaygcscgygarcgyaaycgygtkaarcargtkaayaayggyttygtkaayctgcgycagcatgactcgaccatccgatttttt

SEQ ID NO: 503 - DNA, 85 bp, Artificial Sequence
  l(1)sc_d1-6
trcgytcrcgsgcrttrcgrcgsgcmacrctyggcagytgytcgtgatgatgatggtggtgcatgactcgaccatccgatttttt

SEQ ID NO: 504 - DNA, 85 bp, Artificial Sequence
  l(1)sc_d1-7
tctgtttaacaatcacgcgaatcacctctgaccatatgcaccaccatcatcatcacgarcgcatgactcgaccatccgatttttt

SEQ ID NO: 505 - DNA, 85 bp, Artificial Sequence
  l(1)sc_d2-1
gaygargarctgctggaytayatyagyagytggcargargaaaccgtgcgtttccagtctgcatgactcgaccatccgatttttt

SEQ ID NO: 506 - DNA, 85 bp, Artificial Sequence
  l(1)sc_d2-2
rctratrtartccagcagytcytcrtcrtcyggytgytcrtcrctraarctrtcraarctgcatgactcgaccatccgatttttt

SEQ ID NO: 507 - DNA, 85 bp, Artificial Sequence
  l(1)sc_d2-3
yaarcargarctgcargarcargayctgaarttygayagyttygayagyttyagygaygagcatgactcgaccatccgatttttt

SEQ ID NO: 508 - DNA, 85 bp, Artificial Sequence
  l(1)sc_d2-4
tcytgytcytgcagytcytgyttratrtarccrccrccrctratytcrctrccrctrtargcatgactcgaccatccgatttttt

SEQ ID NO: 509 - DNA, 85 bp, Artificial Sequence
  l(1)sc_d2-5
caragycayagytaycayagygcsagyccracsccragytayagyggyagygaratyagygcatgactcgaccatccgatttttt

SEQ ID NO: 510 - DNA, 85 bp, Artificial Sequence
  l(1)sc_d2-6
rctrtgrtarctrtgrctytgsgcrctytgsgtsgcrccsgtcagraaytgytgrctrctgcatgactcgaccatccgatttttt

SEQ ID NO: 511 - DNA, 85 bp, Artificial Sequence
  l(1)sc_d2-7
gayggyagyagytayaaygaytayaaygayagyctggayagyagycarcarttyctgacsgcatgactcgaccatccgatttttt

SEQ ID NO: 512 - DNA, 85 bp, Artificial Sequence
  l(1)sc_d2-8
artcrttrtarctrctrccrtcrttrctrctytcrtcsgcrctrttrtaratrtgrcgsggcatgactcgaccatccgatttttt

SEQ ID NO: 513 - DNA, 85 bp, Artificial Sequence
  l(1)sc_d2-9
cctagtgcctaaacatatgcaccaccatcatcatcacacscgycayatytayaayagygcgcatgactcgaccatccgatttttt

SEQ ID NO: 514 - DNA, 85 bp, Artificial Sequence
  ase_d1-1
agyctggaraarctgctgggyttygayttyccrccrctggaaaccgtgcgtttccagtctgcatgactcgaccatccgatttttt

SEQ ID NO: 515 - DNA, 85 bp, Artificial Sequence
  ase_d1-2
rcccagcagyttytccagrctrcgratrtaytcmacsgccatrcgcagsgtytcmacyttgcatgactcgaccatccgatttttt

SEQ ID NO: 516 - DNA, 85 bp, Artificial Sequence
  ase_d1-3
gargcscarggygcsggycgyggygcsagyaaraarctgagyaargtkgaracsctgcgygcatgactcgaccatccgatttttt

SEQ ID NO: 517 - DNA, 85 bp, Artificial Sequence
  ase_d1-4
gcrccytgsgcytcraasgcytcrctmacytcytcyggratyttytcrcgcagcagsgcrgcatgactcgaccatccgatttttt

SEQ ID NO: 518 - DNA, 85 bp, Artificial Sequence
  ase_d1-5
cgygarcgyaaycgygtkaarcargtkaayaayggyttygcsctgctgcgygaraaratygcatgactcgaccatccgatttttt

SEQ ID NO: 519 - DNA, 85 bp, Artificial Sequence
  ase_d1-6
tmacrcgrttrcgytcrcgsgcrttrcgrcgsgcmacsgcytgyggcagyggcagrccytgcatgactcgaccatccgatttttt

SEQ ID NO: 520 - DNA, 85 bp, Artificial Sequence
  ase_d1-7
cayaaragycaragygaycaragyttyggyacsccrggycgyaarggyctgccrctgccrgcatgactcgaccatccgatttttt

SEQ ID NO: 521 - DNA, 85 bp, Artificial Sequence
  ase_d1-8
tytgrtcrctytgrctyttrtgyggrtgrctratsgtrccsgcyttyggyggrcgytcmagcatgactcgaccatccgatttttt

SEQ ID NO: 522 - DNA, 85 bp, Artificial Sequence
  ase_d1-9
atcatcatcacaaraartgyaaracsaayaaraarctggcsgtkgarcgyccrccraarggcatgactcgaccatccgatttttt

SEQ ID NO: 523 - DNA, 85 bp, Artificial Sequence
  ase_d1-10
tyttrcayttyttgtgatgatgatggtggtgcatatgccggtgggcgcaaccgatagagtgcatgactcgaccatccgatttttt

SEQ ID NO: 524 - DNA, 85 bp, Artificial Sequence
  dsx_d1-1
cargcscargaygarcarcgygcsctgcayatgcaygargaaaccgtgcgtttccagtctgcatgactcgaccatccgatttttt

SEQ ID NO: 525 - DNA, 85 bp, Artificial Sequence
  dsx_d1-2
gytcrtcytgsgcytgsgcrcgrcgcagsgcsgtytgcagsgccatmacrcgytgrcgrtgcatgactcgaccatccgatttttt

SEQ ID NO: 526 - DNA, 85 bp, Artificial Sequence
  dsx_d1-3
tgyaarttycgytaytgyacstgygaraartgycgyctgacsgcsgaycgycarcgygtkgcatgactcgaccatccgatttttt

SEQ ID NO: 527 - DNA, 85 bp, Artificial Sequence
  dsx_d1-4
trcartarcgraayttrcartarcgyttrtgrccyttcagsgtratyttcagrccrtgrtgcatgactcgaccatccgatttttt

SEQ ID NO: 528 - DNA, 85 bp, Artificial Sequence
  dsx_d1-5
yagyccrcgyacsccrccraaytgygcscgytgycgyaaycayggyctgaaratyacsctgcatgactcgaccatccgatttttt

SEQ ID NO: 529 - DNA, 85 bp, Artificial Sequence
  dsx_d1-6
yggsgtrcgyggrctratrctrctrccrctrctrctrctsgcrccrccrcamacrtcrttgcatgactcgaccatccgatttttt

SEQ ID NO: 530 - DNA, 85 bp, Artificial Sequence
  dsx_d1-7
gaayagygayacsatgagygayagygayatgatygayagyaaraaygaygtktgyggygggcatgactcgaccatccgatttttt

SEQ ID NO: 531 - DNA, 85 bp, Artificial Sequence
  dsx_d1-8
crctcatsgtrtcrctrttccarttytcytcrctmacgtgatgatgatggtggtgcatatgcatgactcgaccatccgatttttt

SEQ ID NO: 532 - DNA, 85 bp, Artificial Sequence
  dsx_d1-9
cagacgtcgttcccgaccatgaggtcatttatattgccatatgcaccaccatcatcatcagcatgactcgaccatccgatttttt

SEQ ID NO: 533 - DNA, 85 bp, Artificial Sequence
  dsx_d2-1
gargargcscgygtkgaratyaaycgyacsgtkgcscargaaaccgtgcgtttccagtctgcatgactcgaccatccgatttttt

SEQ ID NO: 534 - DNA, 85 bp, Artificial Sequence
  dsx_d2-2
ttratytcmacrcgsgcytcytcratrcgrcgrctsgcytcytcratrttsgcrtcsgcrgcatgactcgaccatccgatttttt

SEQ ID NO: 535 - DNA, 85 bp, Artificial Sequence
  dsx_d2-3
ayccrtgggarctgatgccrctgatgtaygtkatyctgaargaygcsgaygcsaayatyggcatgactcgaccatccgatttttt

SEQ ID NO: 536 - DNA, 85 bp, Artificial Sequence
  dsx_d2-4
ggcatcagytcccayggrtarcgraayttytccagcagyttytgrcartartccagraamgcatgactcgaccatccgatttttt

SEQ ID NO: 537 - DNA, 85 bp, Artificial Sequence
  dsx_d2-5
ayggygcsaaygtkccrctgggycargaygtkttyctggaytaytgycaraarctgctgggcatgactcgaccatccgatttttt

SEQ ID NO: 538 - DNA, 85 bp, Artificial Sequence
  dsx_d2-6
gmacrttsgcrccrttyttrcgrttmacrctmacrctratyggcagratsgcsgcrccrcgcatgactcgaccatccgatttttt

SEQ ID NO: 539 - DNA, 85 bp, Artificial Sequence
  dsx_d2-7
garggyagytgygayagyagyagyccragyccragyagyacsagyggygcsgcsatyctggcatgactcgaccatccgatttttt

SEQ ID NO: 540 - DNA, 85 bp, Artificial Sequence
  dsx_d2-8
rctrtcrcarctrccytccagrctytgsgcyggsgtyggmacsgtsgtcatrtgrtgrtcgcatgactcgaccatccgatttttt

SEQ ID NO: 541 - DNA, 85 bp, Artificial Sequence
  dsx_d2-9
atatgcaccaccatcatcatcacagygtkatyacsagygcsgaycaycayatgacsacsggcatgactcgaccatccgatttttt

SEQ ID NO: 542 - DNA, 85 bp, Artificial Sequence
  dsx_d2-10
tgatgatgatggtggtgcatatggtaagtctatcaacaaacgtcatggactgatgttcaagcatgactcgaccatccgatttttt

SEQ ID NO: 543 - DNA, 85 bp, Artificial Sequence
  ovo/svb_d1-1
gcsgtkgayctgagyagyttyctgcarcgyagytgygtkgaaaccgtgcgtttccagtctgcatgactcgaccatccgatttttt

SEQ ID NO: 544 - DNA, 85 bp, Artificial Sequence
  ovo/svb_d1-2
raarctrctcagrtcmacsgcrtcrctcagytgytccagcagrtgrtcsgtytgrctrctgcatgactcgaccatccgatttttt

SEQ ID NO: 545 - DNA, 85 bp, Artificial Sequence
  ovo/svb_d1-3
csgtkgayccrctgcarttyacsgcsacsctgatgctgagyagycaracsgaycayctgcgcatgactcgaccatccgatttttt

SEQ ID NO: 546 - DNA, 85 bp, Artificial Sequence
  ovo/svb_d1-4
tgcagyggrtcmacsgcrccrttyggrcaytgrccrctrctsgccagrtcrctcatratsgcatgactcgaccatccgatttttt

SEQ ID NO: 547 - DNA, 85 bp, Artificial Sequence
  ovo/svb_d1-5
gyagyagyggycarttyaaygcsagygcstaygargaygcsatyatgagygayctggcsagcatgactcgaccatccgatttttt

SEQ ID NO: 548 - DNA, 85 bp, Artificial Sequence
  ovo/svb_d1-6
traaytgrccrctrctrcccatsgtsgcytgratratrttytgsgcraasgtrccratrtgcatgactcgaccatccgatttttt

SEQ ID NO: 549 - DNA, 85 bp, Artificial Sequence
  ovo/svb_d1-7
gaargaygarccrgayatygartaygaygargcsaaratygayatyggyacsttygcscagcatgactcgaccatccgatttttt

SEQ ID NO: 550 - DNA, 85 bp, Artificial Sequence
  ovo/svb_d1-8
cratrtcyggytcrtcyttcagratratrccrtasgcgtgatgatgatggtggtgcatatgcatgactcgaccatccgatttttt

SEQ ID NO: 551 - DNA, 85 bp, Artificial Sequence
  ovo/svb_d1-9
cgttggccgattatatagccggccccgcacggcgcgccatatgcaccaccatcatcatcagcatgactcgaccatccgatttttt

SEQ ID NO: 552 - DNA, 85 bp, Artificial Sequence
  ovo/svb_d2-1
cgyaayatygaytgyatygaygayctgagyaarcayggygaaaccgtgcgtttccagtctgcatgactcgaccatccgatttttt

SEQ ID NO: 553 - DNA, 85 bp, Artificial Sequence
  ovo/svb_d2-2
cratrcartcratrttrcgratrcgrtgrtgrccrccrtgsgcrttytcmacsgccagyggcatgactcgaccatccgatttttt

SEQ ID NO: 554 - DNA, 85 bp, Artificial Sequence
  ovo/svb_d2-3
gayctgcarctggarttygtkaayggyggycayggyatyaaraayccrctggcsgtkgargcatgactcgaccatccgatttttt

SEQ ID NO: 555 - DNA, 85 bp, Artificial Sequence
  ovo/svb_d2-4
tmacraaytccagytgcagrtcyggyggcagrcccagrcgrcgytgcagmacyttrctytgcatgactcgaccatccgatttttt

SEQ ID NO: 556 - DNA, 85 bp, Artificial Sequence
  ovo/svb_d2-5
ggyctgctggcsccragyccracsgtkagygtkctgaaygaragyaargtkctgcarcgygcatgactcgaccatccgatttttt

SEQ ID NO: 557 - DNA, 85 bp, Artificial Sequence
  ovo/svb_d2-6
yggsgccagcagrccgtgatgatgatggtggtgcatatgcgttcggcgggatacctaccagcatgactcgaccatccgatttttt

SEQ ID NO: 558 - DNA, 85 bp, Artificial Sequence
  dfoxo_d2-1
carcarggyttyagygcsgcsagyggyctgccragycargaaaccgtgcgtttccagtctgcatgactcgaccatccgatttttt

SEQ ID NO: 559 - DNA, 85 bp, Artificial Sequence
  dfoxo_d2-2
sgcrctraarccytgytgytgytgrttrcacagsgtcagytcrtcsgccatsgtrccsgtgcatgactcgaccatccgatttttt

SEQ ID NO: 560 - DNA, 85 bp, Artificial Sequence
  dfoxo_d2-3
aayacsacsatgacscargcscaygcscargcsctggargarctgacsggyacsatggcsgcatgactcgaccatccgatttttt

SEQ ID NO: 561 - DNA, 85 bp, Artificial Sequence
  dfoxo_d2-4
cytgsgtcatsgtsgtrttytgrtartcmacyggraarccccartcyggytccagrtcytgcatgactcgaccatccgatttttt

SEQ ID NO: 562 - DNA, 85 bp, Artificial Sequence
  dfoxo_d2-5
yaaygcsagyagytgyggycgyctgagyccratycgygcscargayctggarccrgaytggcatgactcgaccatccgatttttt

SEQ ID NO: 563 - DNA, 85 bp, Artificial Sequence
  dfoxo_d2-6
crcarctrctsgcrttrctrctsgcrcgytgrcgraartcyggrctcagytgraarccrcgcatgactcgaccatccgatttttt

SEQ ID NO: 564 - DNA, 85 bp, Artificial Sequence
  dfoxo_d2-7
accggatggcgccaagcatatgcaccaccatcatcatcacggyggyttycarctgagyccgcatgactcgaccatccgatttttt

SEQ ID NO: 565 - DNA, 85 bp, Artificial Sequence
  ey_d1-1
ggyccrggyccrctggarccrgcscgygcsgcsccrctggaaaccgtgcgtttccagtctgcatgactcgaccatccgatttttt

SEQ ID NO: 566 - DNA, 85 bp, Artificial Sequence
  ey_d1-2
gytccagyggrccyggrccsgcsgcrtgytgsgtrttcagcagrcgcagyttytcrtaragcatgactcgaccatccgatttttt

SEQ ID NO: 567 - DNA, 85 bp, Artificial Sequence
  ey_d1-3
ggygcsagyaayagyggygarggyagygarcargargcsatytaygaraarctgcgyctggcatgactcgaccatccgatttttt

SEQ ID NO: 568 - DNA, 85 bp, Artificial Sequence
  ey_d1-4
crctrttrctsgcrccrccrctytcrctrctrttcagyggsgtsgcsgtytgcatcagrtgcatgactcgaccatccgatttttt

SEQ ID NO: 569 - DNA, 85 bp, Artificial Sequence
  ey_d1-5
kgcsagyggyagycgyggyacsctgagyagyagyacsgayctgatgcaracsgcsacsccgcatgactcgaccatccgatttttt

SEQ ID NO: 570 - DNA, 85 bp, Artificial Sequence
  ey_d1-6
crcgrctrccrctsgcmacrttrctmacrttrccrccratrctmacrctmacyttsgcrcgcatgactcgaccatccgatttttt

SEQ ID NO: 571 - DNA, 85 bp, Artificial Sequence
  ey_d1-7
caccatcatcatcacagyacsagygcsggyaayagyatyagygcsaargtkagygtkagygcatgactcgaccatccgatttttt

SEQ ID NO: 572 - DNA, 85 bp, Artificial Sequence
  ey_d1-8
sgtrctgtgatgatgatggtggtgcatatgcgcccggaactcggccaaaattgcatccatgcatgactcgaccatccgatttttt

SEQ ID NO: 573 - DNA, 85 bp, Artificial Sequence
  ey_d2-1
gayggyggycgyccrgcsggygtkggyctgggyagyggygaaaccgtgcgtttccagtctgcatgactcgaccatccgatttttt

SEQ ID NO: 574 - DNA, 85 bp, Artificial Sequence
  ey_d2-2
gcyggrcgrccrccrtcrccyggmacratrtgrtgrtgsgcyggsgccatyggyggyggrgcatgactcgaccatccgatttttt

SEQ ID NO: 575 - DNA, 85 bp, Artificial Sequence
  ey_d2-3
ygayctgacsccragyagyctgtayccrtgycayatgacsctgcgyccrccrccratggcgcatgactcgaccatccgatttttt

SEQ ID NO: 576 - DNA, 85 bp, Artificial Sequence
  ey_d2-4
grctrctyggsgtcagrtcrccytgytgyggratyggrctyggyggsgccagyggrccmagcatgactcgaccatccgatttttt

SEQ ID NO: 577 - DNA, 85 bp, Artificial Sequence
  ey_d2-5
gytayggygcsgtkacsccratyccragyttyaaycayagygcsgtkggyccrctggcscgcatgactcgaccatccgatttttt

SEQ ID NO: 578 - DNA, 85 bp, Artificial Sequence
  ey_d2-6
gsgtmacsgcrccrtarctrtcrctcatrctcagsgcsgtrtgrtgcatrttrctrtacagcatgactcgaccatccgatttttt

SEQ ID NO: 579 - DNA, 85 bp, Artificial Sequence
  ey_d2-7
gcacccgggcatatgcaccaccatcatcatcacgcsatgtayagyaayatgcaycayacsgcatgactcgaccatccgatttttt

SEQ ID NO: 580 - DNA, 85 bp, Artificial Sequence
  toy_d1-1
atyccrcarccratygcsacsatggcsgaraaytayaaygaaaccgtgcgtttccagtctgcatgactcgaccatccgatttttt

SEQ ID NO: 581 - DNA, 85 bp, Artificial Sequence
  toy_d1-2
gtsgcratyggytgyggratrctrctrtacatsgtrttraarccrctrttcagyggcagrgcatgactcgaccatccgatttttt

SEQ ID NO: 582 - DNA, 85 bp, Artificial Sequence
  toy_d1-3
gygaragyccrccrctgcarccrgcsgcsccrcgyctgccrctgaayagyggyttyaayagcatgactcgaccatccgatttttt

SEQ ID NO: 583 - DNA, 85 bp, Artificial Sequence
  toy_d1-4
gcagyggyggrctytcrctrctsgtrtgsgtsgtrttsgcytcrccrcccagmacsgtyggcatgactcgaccatccgatttttt

SEQ ID NO: 584 - DNA, 85 bp, Artificial Sequence
  toy_d1-5
csctggtkagyaayagyctgccrgargcsagyaayggyccracsgtkctgggyggygarggcatgactcgaccatccgatttttt

SEQ ID NO: 585 - DNA, 85 bp, Artificial Sequence
  toy_d1-6
cytcyggcagrctrttrctmaccagsgcrctrctsgtrcgytcsgcmacrttratsgcrcgcatgactcgaccatccgatttttt

SEQ ID NO: 586 - DNA, 85 bp, Artificial Sequence
  toy_d1-7
aatgcatgcgtaaccttcatatgcaccaccatcatcatcacagygcsatyaaygtkgcsggcatgactcgaccatccgatttttt

SEQ ID NO: 587 - DNA, 85 bp, Artificial Sequence
  toy_d2-1
gtkagygayctgacsggyagyaaytaytggccrcgyctggaaaccgtgcgtttccagtctgcatgactcgaccatccgatttttt

SEQ ID NO: 588 - DNA, 85 bp, Artificial Sequence
  toy_d2-2
trctrccsgtcagrtcrctmacrttytgsgtrctratytgmacyggmacrctmacrccsggcatgactcgaccatccgatttttt

SEQ ID NO: 589 - DNA, 85 bp, Artificial Sequence
  toy_d2-3
csaayagyagyccratgccragyagyaayacsggygtkatyagygcsggygtkagygtkcgcatgactcgaccatccgatttttt

SEQ ID NO: 590 - DNA, 85 bp, Artificial Sequence
  toy_d2-4
ctyggcatyggrctrctrttsgtrtamacrccrtgytgyggyggytgytgrtgsgcsgcrgcatgactcgaccatccgatttttt

SEQ ID NO: 591 - DNA, 85 bp, Artificial Sequence
  toy_d2-5
crtaygtkagygcscaycaycgyaayacsgcstgyaayccragygcsgcscaycarcarcgcatgactcgaccatccgatttttt

SEQ ID NO: 592 - DNA, 85 bp, Artificial Sequence
  toy_d2-6
tgrtgsgcrctmacrtayggrctrcccagrctcagyggrtcrtgraacatrtayggrtasgcatgactcgaccatccgatttttt

SEQ ID NO: 593 - DNA, 85 bp, Artificial Sequence
  toy_d2-7
agyatgacsccragytgyctgcarcarcgygaygcstayccrtayatgttycaygayccrgcatgactcgaccatccgatttttt

SEQ ID NO: 594 - DNA, 85 bp, Artificial Sequence
  toy_d2-8
rcarctyggsgtcatrctrcccagrctrctgtgatgatgatggtggtgcatatggctgcagcatgactcgaccatccgatttttt

SEQ ID NO: 595 - DNA, 85 bp, Artificial Sequence
  stat92e_d2-1
cgygaygtkgcsttyggygarttytayagyaarcgycargaaaccgtgcgtttccagtctgcatgactcgaccatccgatttttt

SEQ ID NO: 596 - DNA, 85 bp, Artificial Sequence
  stat92e_d2-2
craasgcmacrtcrcgyttmacyggrctsgcrttrcgrtcrctyggrtgcagccarcacagcatgactcgaccatccgatttttt

SEQ ID NO: 597 - DNA, 85 bp, Artificial Sequence
  stat92e_d2-3
ycargtkctgaayctggcsgaycgyatycgygayctggaygtkctgtgytggctgcayccgcatgactcgaccatccgatttttt

SEQ ID NO: 598 - DNA, 85 bp, Artificial Sequence
  stat92e_d2-4
gccagrttcagmacytgraartcrcgsgcsgtccayggsgccagcatsgtmaccagrccrgcatgactcgaccatccgatttttt

SEQ ID NO: 599 - DNA, 85 bp, Artificial Sequence
  stat92e_d2-5
ygarctgggyggygtkacsatygcstaygtkaaygaraayggyctggtkacsatgctggcgcatgactcgaccatccgatttttt

SEQ ID NO: 600 - DNA, 85 bp, Artificial Sequence
  stat92e_d2-6
tmacrccrcccagytcrctrtcrctraarcgcagcagraasgtrccratrccrtamacrcgcatgactcgaccatccgatttttt

SEQ ID NO: 601 - DNA, 85 bp, Artificial Sequence
  stat92e_d2-7
tyaayaaracsaargcscaracsgayctgctgcgyagygtktayggyatyggyacsttycgcatgactcgaccatccgatttttt

SEQ ID NO: 602 - DNA, 85 bp, Artificial Sequence
  stat92e_d2-8
tytgsgcyttsgtyttrttratraarcccatratrcarccsgcyttccacatrccgtgatgcatgactcgaccatccgatttttt

SEQ ID NO: 603 - DNA, 85 bp, Artificial Sequence
  stat92e_d2-9
agtttacaaacctgcgagcgttcatatgcaccaccatcatcatcacggyatgtggaargcgcatgactcgaccatccgatttttt

SEQ ID NO: 604 - DNA, 85 bp, Artificial Sequence
  rx_d1-1
gcsatgggycaycaycaygcscayaayggyccrccrccrgaaaccgtgcgtttccagtctgcatgactcgaccatccgatttttt

SEQ ID NO: 605 - DNA, 85 bp, Artificial Sequence
  rx_d1-2
tgrtgrcccatsgcsgccagrctrctcatsgtcagrttrccyggsgccagrctcagyggygcatgactcgaccatccgatttttt

SEQ ID NO: 606 - DNA, 85 bp, Artificial Sequence
  rx_d1-3
yttyctgagycayccrcaracsgtktayccragytayctgacsccrccrctgagyctggcgcatgactcgaccatccgatttttt

SEQ ID NO: 607 - DNA, 85 bp, Artificial Sequence
  rx_d1-4
tgyggrtgrctcagraarccyggcagsgcrctcagcagyggyggrctcagccayggrtcmgcatgactcgaccatccgatttttt

SEQ ID NO: 608 - DNA, 85 bp, Artificial Sequence
  rx_d1-5
ctgccrcaycgyctgggytgyggygcsagyggyctgccrgtkgayccrtggctgagyccrgcatgactcgaccatccgatttttt

SEQ ID NO: 609 - DNA, 85 bp, Artificial Sequence
  rx_d1-6
ccagrcgrtgyggcagytgsgtraartgsgtcagrcccagrcgcagrctytcrctyttytgcatgactcgaccatccgatttttt

SEQ ID NO: 610 - DNA, 85 bp, Artificial Sequence
  rx_d1-7
ccaaatcacgtcatatgcaccaccatcatcatcaccargaraaragygaragyctgcgycgcatgactcgaccatccgatttttt

SEQ ID NO: 611 - DNA, 85 bp, Artificial Sequence
  hbn_d1-1
tayatgggyctgagyaayctgaayggyatyttyggygcsgaaaccgtgcgtttccagtctgcatgactcgaccatccgatttttt

SEQ ID NO: 612 - DNA, 85 bp, Artificial Sequence
  hbn_d1-2
agrttrctcagrcccatrtaytgrctcagcagsgtrtgraarttyggcagyttrtartgygcatgactcgaccatccgatttttt

SEQ ID NO: 613 - DNA, 85 bp, Artificial Sequence
  hbn_d1-3
csgcsgcsgcsggyctgctgccrcarcayctgatggcsccrcaytayaarctgccraaytgcatgactcgaccatccgatttttt

SEQ ID NO: 614 - DNA, 85 bp, Artificial Sequence
  hbn_d1-4
csgcsgcsgcsgcsgcsgcyggrttraartgytgrtgcagsgcraartgyggyggccaragcatgactcgaccatccgatttttt

SEQ ID NO: 615 - DNA, 85 bp, Artificial Sequence
  hbn_d1-5
gccrccrcayggyctgccrggycayccrggyagyatgcaragygarttytggccrccrcagcatgactcgaccatccgatttttt

SEQ ID NO: 616 - DNA, 85 bp, Artificial Sequence
  hbn_d1-6
gcagrccrtgyggyggcagyggratrcccagyggraaytcyggcagrccytgytcyggcagcatgactcgaccatccgatttttt

SEQ ID NO: 617 - DNA, 85 bp, Artificial Sequence
  hbn_d1-7
atcatcatcacttyatgaaycargayaargcsggytayctgctgccrgarcarggyctgcgcatgactcgaccatccgatttttt

SEQ ID NO: 618 - DNA, 85 bp, Artificial Sequence
  hbn_d1-8
trtcytgrttcatraagtgatgatgatggtggtgcatatgctgacatgtaaataagagcggcatgactcgaccatccgatttttt

SEQ ID NO: 619 - DNA, 85 bp, Artificial Sequence
  otp_d1-1
agycaraaygarctgaayggygarccratgccrctgcaygaaaccgtgcgtttccagtctgcatgactcgaccatccgatttttt

SEQ ID NO: 620 - DNA, 85 bp, Artificial Sequence
  otp_d1-2
crttcagytcrttytgrctrctrttyggytgsgcrctcagyggyggyggsgtmacrctrcgcatgactcgaccatccgatttttt

SEQ ID NO: 621 - DNA, 85 bp, Artificial Sequence
  otp_d1-3
gyggyagyccragygcsacsacsccrccraayatgaayagytgyagyagygtkacsccrcgcatgactcgaccatccgatttttt

SEQ ID NO: 622 - DNA, 85 bp, Artificial Sequence
  otp_d1-4
crctyggrctrccrctyggrccrcarctmacrccrccmacrctrtgytgrtacatcatrcgcatgactcgaccatccgatttttt

SEQ ID NO: 623 - DNA, 85 bp, Artificial Sequence
  otp_d1-5
gytggagygtkggygtkaayccratgacsgcsggygayagyatgatgtaycarcayagyggcatgactcgaccatccgatttttt

SEQ ID NO: 624 - DNA, 85 bp, Artificial Sequence
  otp_d1-6
macrccmacrctccarcgrtcrccrccraacatrccsgtrccrcacagrccrtcrcccatgcatgactcgaccatccgatttttt

SEQ ID NO: 625 - DNA, 85 bp, Artificial Sequence
  otp_d1-7
ayggyctgccrccrttyggygcsaayatyacsaayatygcsatgggygayggyctgtgyggcatgactcgaccatccgatttttt

SEQ ID NO: 626 - DNA, 85 bp, Artificial Sequence
  otp_d1-8
craayggyggcagrccrtgrctyggcagcagsgcrccyggsgtrcgraamacrttsgtsggcatgactcgaccatccgatttttt

SEQ ID NO: 627 - DNA, 85 bp, Artificial Sequence
  otp_d1-9
gtacagcgggctccatatgcaccaccatcatcatcacaaracsacsaaygtkttycgyacgcatgactcgaccatccgatttttt

SEQ ID NO: 628 - DNA, 85 bp, Artificial Sequence
  dwg_d1-1
ggygtkggyctgagycaraaragyggytayaaraarcaygaaaccgtgcgtttccagtctgcatgactcgaccatccgatttttt

SEQ ID NO: 629 - DNA, 85 bp, Artificial Sequence
  dwg_d1-2
ytgrctcagrccmacrccrcaratytcrcasgtraasgcyttyttrccsgtrtgrctratgcatgactcgaccatccgatttttt

SEQ ID NO: 630 - DNA, 85 bp, Artificial Sequence
  dwg_d1-3
ayaargcstaytaygayagyagyagyctgcgycarcayaaratyagycayacsggyaaragcatgactcgaccatccgatttttt

SEQ ID NO: 631 - DNA, 85 bp, Artificial Sequence
  dwg_d1-4
rctrctrtcrtartasgcyttrtcrcacagrtcrcasgcrtayggrcgytcrccsgtrtggcatgactcgaccatccgatttttt

SEQ ID NO: 632 - DNA, 85 bp, Artificial Sequence
  dwg_d1-5
ccatcatcatcacgcsttygaycayctgcgycgycayaarctgacscayacsggygarcggcatgactcgaccatccgatttttt

SEQ ID NO: 633 - DNA, 85 bp, Artificial Sequence
  dwg_d1-6
raasgcgtgatgatgatggtggtgcatatggagaccccgccacagcgggagtgtcctgacgcatgactcgaccatccgatttttt

SEQ ID NO: 634 - DNA, 85 bp, Artificial Sequence
  dwg_d2-1
gcscgytgyagyagyttycaygaragycaracscayagygaaaccgtgcgtttccagtctgcatgactcgaccatccgatttttt

SEQ ID NO: 635 - DNA, 85 bp, Artificial Sequence
  dwg_d2-2
ctrctrcarcgsgcytcrtccagrcccagrcgcagcagytcrtcrctrtcratmaccatrgcatgactcgaccatccgatttttt

SEQ ID NO: 636 - DNA, 85 bp, Artificial Sequence
  dwg_d2-3
caytgyctgcarctgctggarcargcsttyaayttyaarcgyatggtkatygayagygaygcatgactcgaccatccgatttttt

SEQ ID NO: 637 - DNA, 85 bp, Artificial Sequence
  dwg_d2-4
agcagytgcagrcartgcagrcaratrtgytgyggcatsgcsgcrccrtcytgyggytcrgcatgactcgaccatccgatttttt

SEQ ID NO: 638 - DNA, 85 bp, Artificial Sequence
  dwg_d2-5
yacsacsctggcsagyatgctgaaytaytgyacsggyctgagyttygarccrcargaygggcatgactcgaccatccgatttttt

SEQ ID NO: 639 - DNA, 85 bp, Artificial Sequence
  dwg_d2-6
ctsgccagsgtsgtrctrccytcrctrcccagrtcratytcytcytgcagyggyttrtgcgcatgactcgaccatccgatttttt

SEQ ID NO: 640 - DNA, 85 bp, Artificial Sequence
  dwg_d2-7
rgtkgtkgtkctgaaytgycgyacstgyacscgygcstgyaarctgcayaarccrctgcagcatgactcgaccatccgatttttt

SEQ ID NO: 641 - DNA, 85 bp, Artificial Sequence
  dwg_d2-8
grcarttcagmacmacmacytcsgcratyttrctrttgtgatgatgatggtggtgcatatgcatgactcgaccatccgatttttt

SEQ ID NO: 642 - DNA, 85 bp, Artificial Sequence
  dwg_d2-9
ggttatcatggtagtctttagcaaaaggtgcgatcagcatatgcaccaccatcatcatcagcatgactcgaccatccgatttttt

SEQ ID NO: 643 - DNA, 19 bp, Artificial Sequence
  nSDA Primer
tcggatggtcgagtcatgc

SEQ ID NO: 644 - DNA, 25 bp, Artificial Sequence
  lacz-f
aatttcacacaggaaacagctatga

SEQ ID NO: 645 - DNA, 18 bp, Artificial Sequence
  LacZ-R
cgctcagtggtggtgatg

SEQ ID NO: 646 - DNA, 25 bp, Artificial Sequence
  rfp-f
aatttcacacaggaaacagctatga

SEQ ID NO: 647 - DNA, 19 bp, Artificial Sequence
  rfp-r
agactggaaacgcacggtt

SEQ ID NO: 648 - DNA, 39 bp, Artificial Sequence
  rfp-m
tgttgttaccgttacccaggactcctccctgcaagacgg

SEQ ID NO: 649 - DNA, 22 bp, Artificial Sequence
  tf-f
catatgcaccaccatcatcatc

SEQ ID NO: 650 - DNA, 19 bp, Artificial Sequence
  tf-r
agactggaaacgcacggtt

SEQ ID NO: 651 - DNA, 85 bp, Artificial Sequence; identical to SEQ ID NO: 13
  rfp-f1-1

SEQ ID NO: 652 - DNA, 85 bp, Artificial Sequence; identical to SEQ ID NO: 14
  rfp-f1-2

SEQ ID NO: 653 - DNA, 85 bp, Artificial Sequence; identical to SEQ ID NO: 15
  rfp-f1-3

SEQ ID NO: 654 - DNA, 85 bp, Artificial Sequence; identical to SEQ ID NO: 16
  rfp-f1-4

SEQ ID NO: 655 - DNA, 85 bp, Artificial Sequence; identical to SEQ ID NO: 17
  rfp-f1-5

SEQ ID NO: 656 - DNA, 85 bp, Artificial Sequence; identical to SEQ ID NO: 18
  rfp-f1-6

SEQ ID NO: 657 - DNA, 85 bp, Artificial Sequence; identical to SEQ ID NO: 19
  rfp-f1-7

SEQ ID NO: 658 - DNA, 85 bp, Artificial Sequence; identical to SEQ ID NO: 20
  rfp-f1-8

SEQ ID NO: 659 - DNA, 85 bp, Artificial Sequence; identical to SEQ ID NO: 21
  rfp-f1-9

SEQ ID NO: 660 - DNA, 85 bp, Artificial Sequence; identical to SEQ ID NO: 22
  rfp-f2-1

SEQ ID NO: 661 - DNA, 85 bp, Artificial Sequence; identical to SEQ ID NO: 23
  rfp-f2-2

SEQ ID NO: 662 - DNA, 85 bp, Artificial Sequence; identical to SEQ ID NO: 24
  rfp-f2-3

SEQ ID NO: 663 - DNA, 85 bp, Artificial Sequence; identical to SEQ ID NO: 25
  rfp-f2-4

SEQ ID NO: 664 - DNA, 85 bp, Artificial Sequence; identical to SEQ ID NO: 26
  rfp-f2-5

SEQ ID NO: 665 - DNA, 85 bp, Artificial Sequence; identical to SEQ ID NO: 27
  rfp-f2-6

SEQ ID NO: 666 - DNA, 85 bp, Artificial Sequence; identical to SEQ ID NO: 28
  rfp-f2-7

SEQ ID NO: 667 - DNA, 85 bp, Artificial Sequence; identical to SEQ ID NO: 29
  rfp-f2-8

SEQ ID NO: 668 - DNA, 85 bp, Artificial Sequence; identical to SEQ ID NO: 30
  rfp-f2-9

SEQ ID NO: 669 - DNA, 25 bp, Artificial Sequence
  rfp-f
aatttcacacaggaaacagctatga

SEQ ID NO: 670 - DNA, 19 bp, Artificial Sequence
  rfp-r
agactggaaacgcacggtt

SEQ ID NO: 671 - DNA, 39 bp, Artificial Sequence; identical to SEQ ID NO: 648
  rfp-m